(19) United States
(12) Patent Application Publication
(10) Pub. No.: US 2023/0263801 A1
(43) Pub. Date: Aug. 24, 2023

(54) COMBINATION THERAPY OF PARP INHIBITORS

(71) Applicant: IMPACT THERAPEUTICS (SHANGHAI), INC, Shanghai (CN)

(72) Inventors: Sui Xiong CAI, Shanghai (CN); Ye Edward TIAN, Shanghai (CN)

(21) Appl. No.: 18/006,603

(22) PCT Filed: Jul. 23, 2021
(86) PCT No.: PCT/CN2021/108192

§ 371 Date: Jan. 24, 2023

(30) Foreign Application Priority Data
Jul. 24, 2020 (CN) 202010722483.1

(51) Int. Cl. A61K 31/517; A61P 35/00; A61K 45/06

(52) U.S. Cl. CPC A61K 31/517; A61P 35/00; A61K 45/06

(57) ABSTRACT

The present disclosure relates to method of combination therapies of PARP inhibitors. Specifically, the present disclosure relates to combination therapies of a PARP inhibitor represented by the following Formula (I) and a low dose of a DNA damaging anti-cancer drug. Adopting the combination therapy of the present disclosure can reduce the daily dose of DNA damaging anti-cancer drugs, reduce toxicity and improve anti-cancer effects.

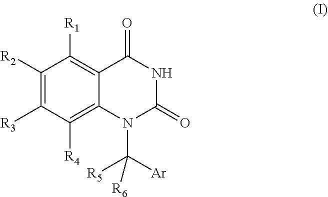

### FIELD OF THE DISCLOSURE

The present disclosure relates to method of combination therapies of PARP inhibitors.

### BACKGROUND OF THE INVENTION

Poly (ADP-ribose) polymerase (PARP) catalyzes intracellular ADP-ribose polymerization reactions. It adds ADP-ribose to target protein molecules by consuming NAD+. PARP is a family of enzymes comprising 17 family members PARP1 and PARP2 are critical players in DNA damage repair pathways and part of the base excison repair (BER) complex. Upon activation PARP1 and PARP2 attach poly (ADP-ribose) (PAR) to proteins such as histones as well as to themselves. The reaction is referred to as PARylation. The DNA repair mechanism is essential for maintaining DNA stability and chromosome integrity, and therefore ensuring the survival of mammalian cells. Scientific evidence indicates that cells with DNA damage repair pathway defects such as defects in homologous recombination are sensitive to PARP1 inhibition. Several PARP inhibitors have been developed and approved for the treatment of cancers that have BRCA mutations or homologous recombination defects.

All PARP inhibitors approved up to now are not selective between PARP1 and PARP2. For instance, Olaparib inhibits PARP1 and PARP2 with similar potency. The applications of PARP inhibitors in the treatment of cancers are mainly two types, used as monotherapy or used in combination with other anti-cancer agents. First, for cancers with DNA repair deficiency, such as triple-negative breast cancers with BRCA1 or BRCA2 mutations, PARP inhibitors can be used as monotherapy to kill cancer cells directly through the mechanism of synthetic lethality. According to statistics, about 10-15% of breast cancer patients are due to family inherited factors, in which the BRCA1 or BRCA2 gene mutations account for 15-20% of all hereditary breast cancers. Second, cancer cells usually have much higher DNA mutation rate that leads to higher degree of chromosomal instability than normal cells. These cancer cells are sensitive to drugs that cause DNA damage, such as DNA alkylating/methylating agents and topoisomerase I inhibitors. However, because of the existence of DNA repair pathways, the therapeutic effects of these drugs can not be fully realized. Inhibition of DNA repair mechanism such as with PARP inhibitors can dramatically improve the therapeutic efficacy of DNA-damaging chemotherapy drugs such as Temozolomide (TMZ) Synergistic effects have been observed with PARP inhibitors in combination with TMZ in pre-clinical pharmacological models. High dose TMZ and low dose PARP inhibitors are commonly used in combination studies in pre-clinical models. For instance, in an efficacy study a low-dose of Olaparib (10 mg/kg per day, the single effective dose of Olaparib (AZD2281) in BRCA mutant animal model study was 50-100 mg/kg per day) was combined with a high-dose TMZ (50 mg/kg per day) in a human colon cancer SW620 xenograft model (Keith A. Menear et al. 2008, JMC 51:6581). In clinical trials, high-dose chemotherapeutic anti-cancer drugs are also usually combined with low-dose PARP inhibitors. For example, in a Phase I clinical combination trial of Olaparib with TMZ in patients with recurrent glioblastoma, Olaparib was found to exacerbate TMZ-related hematological toxicity, necessitating intermittent dosing. Of 36 patients evaluated for efficacy, 14 (39%) remained progression free at 6 months. The recommended Phase 11 dose (RP2D) was Olaparib 150 mg 3 days/week (the approved dose of Olaparib as a single agent is 400 mg twice a day (BID) continuously) and TMZ 75 mg/m² daily for 42 days (TMZ 75 mg/m², equivalent to 121.5 mg once a day (QD) to the body surface area of 60 kg adult 1.62 m²) (Catherine Hanna et al. 2020, Neuro Oncol. 1-11). These doses of TMZ are similar to the daily dose of TMZ in combination with radiotherapy for newly diagnosed glioblastoma multiforme, which is 75 mg/m², and close to the lowest recommended dose of TMZ as monotherapy, which is 100 mg/m² (equivalent to 162 mg).

It has been widely accepted that the mechanism of action of PARP inhibitors to kill cancer cells is beyond the original hypothesis that PARP inhibitors inhibit DNA repair. PARP inhibitors can stabilize PARP-DNA complex at single-strand DNA break site, that is referred to as “trapping”. Trapping has been considered to cause much more cytotoxicity than inhibiting single-strand break repair, therefore considered to be closedly related to therapeutic efficacy (Murai and Pommier 2019 Annu Rev Cancer Biol 3:7.1-7.20). Accordingly, there are two approaches to apply the combination of PARP inhibitor with DNA damaging alkylating agent, such as TMZ. One approach is to use TMZ as the major cause of DNA damage and PARP inhibitor acting as a potentiating agent by blocking the DNA repair mechanism. The mechanism is to cause more DNA damage with TMZ in the presence of PARP inhibitor. The other approach is relying on trapping activity of PARP inhibitor where TMZ acts as a priming agent to induce DNA lesions, and PARP inhibitor acts as trapping agent to produce DNA-PARP complex. Since trapping can cause more severe cytotoxicity to cancer cells, this is considered as a more rational approach (Murai J. et al. 2014, J Pharmacol Exp Ther 349:408). The major difference between these two approaches is in dose selection and dosing schedule. Using this novel combination strategy, TMZ may be effective at much reduced dosages to minimize its toxicity, and PARP inhibitors may be dosed at levels approaching their maximum tolerated doses to produce the maximal trapping and antitumor efficacy (Shen Y. et al. 2015, J Pharmacol Exp Ther 353:446). An additional advantage of this combination approach is that it is not dependent on specific mutation, such as BRCA mutation for efficacy, and could be used for the treatment of patients with different types of cancer.

Combination of high-dose PARP inhibitor with potent PARP-trapping activity and low-dose TMZ has been reported. In a pre-clinical efficacy study of human small cell lung cancer (SCLC) NCI-H209 xenograft model, a high-dose Talazoparib (0.25 mg/kg, single agent effective dose of Talazoparib in BRCA mutated model is 0.33 mg/kg) was combined with a low-dose TMZ (3 mg/kg, the effective dose of TMZ as a single agent in the human colon cancer SW620 xenograft model is reported to be 50 mg/kg, Keith A. Menear et. al. 2008, JMC 51:6581) administered in an intermittent daily dosing schedule (D1-4, D17-20 and D28-31), and was reported to produce good synergetic efficacy with some toxicity (about 10% body weight reduction was observed after the first QD4 dosing) (Feng et al., EORTC2014, Ab #242). In a clinical setting, a Phase I/II combination clinical study of Talazoparib and TMZ was reported (Wainberg el al., AACR2016, CT011). In that study, the starting dose and schedule were Talazoparib 0.5 mg (QD for D1-28, the approved dose of Talazoparib as a single agent is 1 mg QD continuosly) and TMZ 25 mg/m² (QD for DI-5) in a 28-day cycle. The maximum tolerated dose (MTD) was determined to be Talazoparib 1 mg (QD for D1-28) and TMZ 37.5 mg/m² (QD for D1-5, 37.5 mg/m² is equal to 65.6 mg) of each 28-day cycle 2 PR (partial response) as well as 2 SD was observed in non-BRCA mutated ovarian cancer patients, and days on study treatment of over 200 days were observed in 5 patients with melanoma, cholagiocarcinoma and ovarian cancer.

A Phase I/II combination clinical study of Olaparib and TMZ for the treatment of small cell lung cancer (SCLC) has been reported. The RP2D is Olaparib 200 mg BID (the approved dose of Olaparib as a single agent is 300 or 400 mg BID) and TMZ 75 mg/m² QD (equivalent to 121.5 mg QD), both on days 1-7 of a 21-day cycle (Farago et al., 2019, Cancer Discov 9:1372). The confirmed overall response rate was reported to be 41.7% (20/48 evaluable); and median progression-free survival was 4.2 months and median overall survival was 8.5 months. In addition, PDX models generated from the patients in the clinical trial were used to explore multiple doses and dosing schedule for Olaparib combined with TMZ, which included intermittent, continuous, sequential and alternating dosing. The dosages of Olaparib were 25 mg/kg and 12.5 mg/kg BID; the dosages of TMZ were 12.5 mg/kg, 6.25 mg/kg, 3.13 mg/kg and 1.56 mg/kg. Based on the PDX model studies, it was concluded that continuous Olaparib combined with intermittent TMZ is a good dosing schedule for human studies. A new cohort for continuous Olaparib and intermittent TMZ was added to the ongoing Phase I/II clinical trial, in which the starting dose and schedule was Olaparib 50 mg BID continuously for D1-21 and TMZ 50 mg/m² (equivalent to 87.5 mg) QD continuously for DI-7 of each 21-day cycle (Drapkin et al., AACR 2019, Abstract 4736).

In order to achieve good efficacy and good tolerability in combination therapy, more research is needed to identify the appropriate dosage and dosing schedule of PARP inhibitor with good trapping activity combined with the appropriate dosage and dosing schedule of DNA damaging anticancer drug, such as TMZ, for the treatment of cancers.

### SUMMARY OF THE DISCLOSURE

The present disclosure relates to a combination therapy with one or more PARP inhibitors, especially compounds disclosed in PCT/CN2012/073362 (corresponding to U.S. Pat. No. 9,290,460), and one or more DNA damaging anti-cancer drugs, such as TMZ, for the treatment of cancer.

Specifically, the present disclosure relates to a combination therapy of one or more PARP inhibitors represented by Formula I, II or III described herein and one or more DNA damaging anti-cancer drugs, especially TMZ, which includes the use thereof in the manufacture of a medicament for the treatment or prevention of PARP-mediated diseases or diseases that benefit from treatment with DNA damage agent.

The present disclosure also provides a method for treating tumors, comprising administering an effective dose of one or more PARP inhibitors with good PARP-trapping activity described herein with a low dose of one or more DNA damaging anti-cancer drugs such as TMZ to a subject in need thereof.

The present disclosure also provides a combination of one or more PARP inhibitors with good PARP-trapping activity with one or more DNA damaging anti-cancer drugs such as TMZ for use in a method for treating cancers.

Also provided is a kit containing a pharmaceutical preparation of a PARP inhibitor represented by Formula I, II or III described herein and a pharmaceutical preparation of a DNA damaging anti-cancer drug, especially of TMZ. In some embodiments, in the kit, the content of the PARP inhibitor in the pharmaceutical preparation of the PARP inhibitor meets a requirement on an effective daily dose of the PARP inhibitor when it is administered alone; in the pharmaceutical preparation of the DNA damaging anti-cancer drug, the content of the DNA damaging anti-cancer drug can provide a daily dose of about 1/12th to about 115th of the daily dose by weight of the DNA damaging anti-cancer drug administered alone or in combination with other drugs or therapies. The kit can contain one or more doses of the pharmaceutical preparation of the PARP inhibitor and one or more doses of the pharmaceutical preparation of the DNA damaging anti-cancer drug, so as to meet the requirement of administering a patient one or more days of drugs for one or more days of treatment. It should be understood that the one dose can be one or more tablets or other forms of pharmaceutical preparations, as long as the total amount of active ingredients (i.e. PARP inhibitors or DNA damaging anti-cancer drugs) in the one or more tablets meets the requirements of dosage.

In some embodiments, the kit comprises a pharmaceutical preparation of 5-fluoro-1-(4-fluoro-3-(4-(pyrimidin-2-yl)piperazine-1-carbonyl)benzyl)quinazoline-2,4(1H,3H)-dione (IMP4297, senaparib) and a pharmaceutical preparation of TMZ, which are packaged independently, wherein in the pharmaceutical preparation of 5-fluoro-1-(4-fluoro-3-(4-(pyrimidin-2-yl)piperazine-1-carbonyl)benzyl)quinazoline-2,4(1H,3H)-dione, the content of 5-fluoro-1-(4-fluoro-3-(4-(pyrimidin-2-yl)piperazine-1-carbonyl)benzyl)quinazoline-2,4(1H,3H)-dione meets the daily dose requirement of 20-120 mg, and in the pharmaceutical preparation of TMZ, the content of TMZ meets the daily dose requirement of 10-30 mg.

The present disclosure also provides a compound preparation, which contains one or more PARP inhibitors represented by Formula I, II or III described herein and one or more DNA damaging anti-cancer drug. The content of the PARP inhibitor and the DNA damaging anti-cancer drugs in the compound preparation is as defined in the above-mentioned kit's embodiments.

In some embodiments, the compound preparation comprises 5-fluoro-1-(4-fluoro-3-(4-(pyrimidin-2-yl)piperazine-1-carbonyl)benzyl)quinazoline-2,4(1H,3H)-dione and TMZ, wherein in the compound preparation the content of 5-fluoro-1-(4-fluoro-3-(4-(pyrimidin-2-yl)piperazine-1-carbonyl)benzyl)quinazoline-2,4(1H,3H)-dione meets the daily dose requirement of 20-120 mg, and the content of TMZ meets the daily dose requirement of 10-30 mg.

Also provided is the use of 5-fluoro-1-(4-fluoro-3-(4-(pyrimidin-2-yl)piperazine-1-carbonyl)benzyl)quinazoline-2,4(1H,3H)-dione and TMZ or a compound preparation containing them in the preparation of a medicament for treatment of cancer.

In one or more embodiments, the invention provides the dosages and dosing schedule for the combination of 5-fluoro-1-(4-fluoro-3-(4-(pyrimidin-2-yl)piperazine-1-carbonyl)benzyl)quinazoline-2,4(1H,3H)-dione (senaparib) and TMZ for use in the treatment of cancer. The dosage of senaparib is 20-100 mg once a day, continuously for 28 days, and the dosage of TMZ is 10-30 mg once a day, continuously for 21 days of a 28-day cycle.

In one or more embodiments, the tumor or cancer described in the disclosure includes liver cancer, melanoma, Hodgkin's disease, non-Hodgkin's lymphoma, acute lymphocytic leukemia, chronic lymphocytic leukemia, multiple myeloma, neuroblastoma, breast cancer, ovarian cancer, non-small cell lung cancer, small cell lung cancer, Wilms tumor, cervical cancer, testicular cancer, soft tissue sarcoma, primary macroglobulinemia, bladder cancer, chronic myeloid leukemia, primary brain cancer, malignant melanoma, gastric cancer, colon cancer, malignant pancreatic islet tumor, malignant carcinoid cancer, choriocarcinoma, mycosis fungoides, head and neck cancer, osteogenic sarcoma, pancreatic cancer, acute myeloid leukemia, hairy cell leukemia, rhabdomyosarcoma, Kaposi's sarcoma, urogenital tumors, thyroid cancer, esophageal cancer, malignant hypercalcemia, cervical hyperplasia, renal cell carcinoma, endometrial cancer, polycythemia vera, idiopathic thrombocythemia, adrenocortical carcinoma, skin cancer and prostate cancer.

### DETAILED DESCRIPTION OF THE DISCLOSURE

It should be understood that, within the scope of the present disclosure, the above technical features of the present disclosure and the technical features specifically described in the following (e.g., Examples) can be combined with each other, thereby forming technical solution(s).

It is disclosed in this invention that low-dose DNA damaging anti-cancer drugs, such as alkylating agent anti-cancer drugs, such as TMZ, are used as a priming agent to induce DNA lesions and a PARP-DNA complex is formed via combination with an effective high dose of PARP inhibitor with potent PARP-trapping activity, such as 5-fluoro-1-(4-fluoro-3-(4-(pyrimidin-2-yl)piperazine-1-carbonyl)benzyl)quinazoline-2,4(1H,3H)-dione (IMP4297, senaparib). This combination of these two kinds of drugs produces high anti-cancer efficacy with low toxicity. Therefore, the disclosure provides a combined medication method for treating cancer, comprising administering an effective dose of one or more PARP inhibitors with potent PARP-trapping activity, especially IMP4297, and a low dose of one or more DNA damaging anti-cancer drug, such as TMZ. It was discovered by the inventors that in the xenograft model of SCLC, the continuous dosing of low dose of TMZ (3 mg/kg daily D1-21, the effective dose of TMZ as a single agent in the human colon cancer SW620 xenograft model is reported to be 50 mg/kg, Keith A Menear el al. 2008, JMC 51:6581) combined with effective dose of IMP4297 (5 mg/kg or 10 mg/kg, daily D1-21, the effective dose of IMP4297 as a single agent in animal model with BRCA mutation is 2.5-20 mg/kg per day, 8-fold of therapeutic window) shows extremely strong synergistic effect, and no more than 5% body weight loss as well as no significant hemocological changes in all drug-treated groups. This combination of low dose TMZ with effective high dose IMP4297, which is dosed daily (i.e., continuously), has good efficacy and is well tolerated in vivo. In comparison, in the efficacy study employing high dose Talazoparib combined with low dose TMZ using the same dose of 3 mg/kg TMZ once daily in the same SCLC xenograft model, with an intermittent dosing schedule (D1-4, D17-20 and D28-31), about 10% body weight reduction was observed after the first QD4 dosing (Feng et al., EORTC2014, Ab #242).

Based on the results of animal model studies, as well as the clinical studies of IMP4297 as a single agent for the treatment of patients with cancer, which showed that the clinically effective dose of IMP4297 as a single agent in cancer patients was 20-120 mg (6-fold of therapeutic window) QD continuously (Xu B el al, 2020 ESMO, abstract #1317, and Souza P et al. 2020 ESMO, abstract #1338), a Phase I/II combination clinical study of effective high dose IMP4297 with low dose TMZ was initiated (ClinicalTrials.gov Identifier: NCT04434482) and is ongoing (Cohort 0, IMP4297 40 mg+TMZ 10 mg; Cohort 1: IMP4297 40 mg+TMZ 20 mg; Cohort 2: IMP4297 60 mg+TMZ 20 mg; Cohort 3: IMP4297 80 mg+TMZ 20 mg; Cohort 4: IMP4297 80 mg+TMZ 30 mg, the RP2D of IMP4297 as a single agent is 100 mg QD continuously) The dosing schedule is IMP4297 QD continuously for days 1-28 and TMZ QD continuously for days 1-21 of a 28-day cycle Preliminary clinical data indicated good tolerability and good efficacy in patients with different types of advanced solid tumors. The dosage and dosing schedule of TMZ in the clinical study of IMP4297 combined with TMZ are different from the clinical study of Olapaib combined with TMZ, as well as different from the clinical study of Talazoparib combined with TMZ, in that both the Olaparib with TMZ and Talazoparib with TMZ studies used a relatively high dose of TMZ with an intermittent dosing schedule.

The combination of an effective high dose IMP4297 (20-100 mg QD), which has potent PARP-trapping activity as well as a large therapeutic window (2.5-20 mg/kg QD in mice with 8-fold of therapeutic window and 20-120 mg QD in human with 6-fold of therapeutic window), with a low dose TMZ (10-30 mg QD), using a dosing schedule of IM P4297 QD continuously for days 1-28 and TMZ QD continuously for days 1-21 of a 28-day cycle, gave good tolerability and good efficacy in patients with different types of advanced solid tumors. In some embodiments, the dosage of TMZ is 10-30 mg QD and the dosage of LMV4297 is 20-100 mg QD, the dosing schedule of IMP4297 is QD continuously for 28 days, and TMZ is QD continuously for 21 days of a 28-day cycle. In some embodiments, the dosage of TMZ is 20-30 mg QD and the dosage of IMP4297 is 40-100 mg QD. In another embodiment, the dosage of TMZ is 20 mg QD and the dosage of IMP4297 is 40, 60 or 80 mg QD. In another embodiment, the dosage of TMZ is 30 mg QD and the dosage of IMP4297 is 80 mg QD. In another embodiment, the dosage of TMZ is 10 mg QD and the dosage of IMP4297 is 40 mg QD. In another embodiment, the dosage of TMZ is 10 mg QD and the dosage of IMP4297 is 20 mg QD.

Since this combination approach is not dependent on specific mutation, such as BRCA mutation in cancer for efficacy, one advantage of this combination is that it could be used for the treatment of patients with different types of cancer.

By the term “low dose” according to the invention is intended a dose that is about 1/12th to about ⅕th of the lowest recommended dose that is approved or will be approved for the DNA damaging drug such as TMZ, by the U.S. Food and Drug Administration (FDA), the European Medicines Agency (EMA), the National Medical Products Administration (NMPA) and the Pharmaceutical and Medical Devices Agency (PMDA), as the following table.

| | Lowest Approved | | |
|---|---|---|---|
| | Dose (mg/m²) by | Lowest | |
| | FDA/EMA/NMPA/ | Approved | Low Dose |
| Therapy | PMDA | Dose (mg)* | (mg) |
| Concomitant with | 75 | 121.5 | 10.125-24.3 |
| radiotherapy | | | |
| Monotherapy | 100 | 162 | 13.5-32.4 |

Note:
*Conversion based on the body surface area of 60 kg adult 1.62 m².

By the term “about” is +10% of the recited amount. For example, about 10 is intended to mean 9-11, inclusive.

PARP Inhibitors

The PARP inhibitors described in the disclosure especially include the PARP inhibitors disclosed in PCT/CN2012/073362 (corresponding to U.S. Pat. No. 9,290,460), the full texts of which are incorporated herein by reference. The PARP inhibitors of the disclosure also include those compounds which are patented in CN 103097361 B, CN 104230827 B and EP 2 709 990 BI, the full texts of which are incorporated herein by reference.

Specifically, PARP inhibitors of the present disclosure are selected from compounds represented by Formula I:

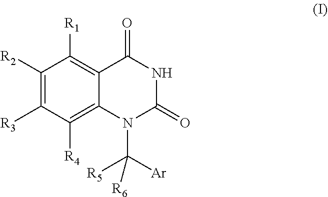

or pharmaceutically acceptable salts, solvates or prodrugs thereof, wherein:

Ar is an optionally substituted aryl or an optionally substituted heteroaryl;

R₁-R₆ are independently hydrogen, halo, optionally substituted amino, optionally substituted alkoxy, optionally substituted C₁₋₁₀, alkyl (such as haloalkyl, hydroxylalkyl, aminoalkyl, carboxylalkyl), alkenyl, alkynyl, nitro, cyano, acylamido, hydroxy, thiol, acyloxy, azido, carboxy, ethylenedioxo, hydroxylamido or optionally substituted alkylthiol.

Compounds of Formula I include compounds wherein Ar is an optionally substituted phenyl, pyridyl or furanyl. In some embodiments, Ar is phenyl, pyridyl or furanyl, substituted with a substituted carbonyl or methyl, preferably carbonyl, at the meta-position. In some embodiments, R₅ and R₆ are hydrogen.

In some embodiments of compounds of Formula I, Ar is phenyl, pyridyl or furanyl, in some embodiments, phenyl, substituted with a substituted carbonyl at the meta-position; R₁ is halo, NH₂, C₁₋₆ alkyl, nitro or hydroxyl, R₂-R₄ are independently hydrogen, halo, NH₂, C₁₋₆ alkyl, nitro or hydroxy; or R₂ is halo, NH₂, C₁₋₆ alkyl, nitro or hydroxyl, R₁ and R₃-R₄ are independently hydrogen, halo, NH₂, C₁₋₆ alkyl, nitro or hydroxyl; or R₃ is halo, NH₂, C₁₋₆ alkyl, nitro or hydroxyl, R₁, R₂ and R₄ are independently hydrogen, halo, NH₂, C₁₋₆ alkyl, nitro or hydroxyl; or R₄ is halo, NH₂, C₁₋₆ alkyl, nitro or hydroxyl, R₁-R₃ are independently hydrogen, halo, NH₂, C₁₋₆ alkoxy, C₁₋₆ alkyl, nitro or hydroxyl; R₅ and R₆ are hydrogen; wherein, the substituted carbonyl is a carbonyl substituted by one substituent selected from the following: piperazinyl or piperidinyl, optionally substituted by one substituent selected from the following, pyridinyl, pyrimidinyl, C₃₋₈ cycloalkyl, C₁₋₆ alkyl optionally substituted by one C₃₋₈ cycloalkyl, benzoyl optionally substituted by one or more substituents selected from halogen and C₁₋₆ alkoxy, carbonyl optionally substituted by one substituent selected from C₃₋₈ cycloalkyl, thiophenyl, pyridinyl, furanyl and tetrahydrofuranyl, C₁₋₆ alkylsulfonyl, phenyl, pyrazinyl, benzo[d]isothiazol-3-yl, benzoisoxazolyl optionally substituted by one or more halogens, thiazolyl, piperidinyl, and phenoxy.

In some embodiments, PARP inhibitors of the present disclosure are selected from compounds represented by Formula II:

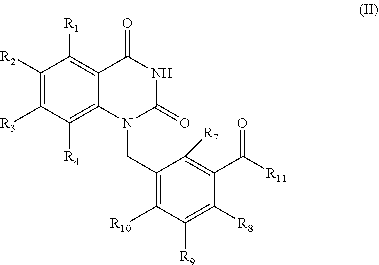

or pharmaceutically acceptable salts, solvates or prodrugs thereof, wherein:

R₁-R₄ are independently hydrogen, halo, optionally substituted amino, optionally substituted alkoxy, optionally substituted C₁₋₁₀ alkyl (such as haloalkyl, hydroxylalkyl, aminoalkyl, and carboxylalkyl), alkenyl, alkynyl, nitro, cyano, acylamido, hydroxy, thiol, acyloxy, azido, carboxy, ethylenedioxo, hydroxyamido or optionally substituted alkylthiol;

R₇-R₁₀ are independently hydrogen, halo, optionally substituted amino, alkoxy, C₁₋₁₀ alkyl, haloalkyl, aryl, heteroaryl, carbocyclic group, heterocyclic group, alkenyl, alkynyl, arylalkyl, arylalkenyl, arylalkynyl, heteroarylalkyl, heteroarylalkenyl, heteroarylalkynyl, carbocycloalkyl, heterocycloalkyl, hydroxyalkyl, hydroxyalkoxy, aminoalkyl, aminoalkoxy, carboxyalkyl, carboxyalkoxy, nitro, cyano, acylamido, aminocarbonyl, hydroxy, thiol, acyloxy, azido, carboxy, carbonylamido, alkylsulfonyl, aminosulfonyl, di-substituted alkylaminosulfonyl, alkylsulfiniyl, alkylthiol, or substituted carbonyl;

R₁₁ is an optionally substituted amino, hydrazine, alkoxy, C₁₋₁₀ alkyl, haloalkyl, aryl, heteroaryl, carbocyclic group, heterocyclic group, alkenyl, alkynyl, arylalkyl, arylalkenyl, arylalkynyl, heteroarylalkyl, heteroarylalkenyl, heteroarylalkynyl, carbocycloalkyl, heterocycloalkyl, hydroxyalkyl, hydroxyalkoxy, aminoalkyl, aminoalkoxy, carboxyalkyl, carboxyalkoxy, acylamido, hydroxy, thiol, acyloxy, hydroxylamido, or alkylthiol.

In some embodiments of compounds of Formula II, R₇, R₈, R₉ and R₁₀ are independently hydrogen or halo, in some embodiments fluoro. In some embodiments, in compounds of Formula II, R₁ and R₂ are independently hydrogen, fluoro, chloro, bromo or methyl. In some embodiments, in compounds of Formula II, R₄ is hydrogen, fluoro, methyl, methoxy or hydroxy. In some embodiments, in compounds of Formula II, R₁₁ is substituted amino, in some embodiments, substituted piperazinyl or piperidinyl.

In some embodiments of compounds of Formula II, R₁ is halo, NH₂, C₁₋₆ alkyl, nitro or hydroxyl, R₂-R₄ are independently hydrogen, halo, NH₂, C₁₋₆ alkyl, nitro or hydroxyl; or R₂ is halo, NH₂, C₁₋₆ alkyl, nitro or hydroxyl, R₁ and R₃-R₄ are independently hydrogen, halo, NH₂, C₁₋₆ alkyl, nitro or hydroxyl; or R₃ is halo, NH₂, C₁₋₆ alkyl, nitro or hydroxyl, R₁, R₂ and R₄ are independently hydrogen, halo, NH₂, C₁₋₆ alkyl, nitro or hydroxyl; or R₄ is halo, NH₂, C₁₋₆ alkyl, nitro or hydroxyl, R₁-R₃ are independently hydrogen, halo, NH₂, C₄ alkyl, nitro or hydroxyl, R₇-R₁₀ are independently hydrogen, halo, NH₂, C₁₋₆ alkoxy, C₁₋₆ alkyl, halogenated C₁₋₆ alkyl and nitro; R₁₁ is selected from the following, piperazinyl or piperidinyl, optionally substituted by one substituent selected from the following: pyridinyl, pyrimidinyl, C₃₋₈ cycloalkyl, C₁₋₆ alkyl optionally substituted by one C₃₋₈ cycloalkyl, benzoyl optionally substituted by one or more substituents selected from halogen and C₁₋₆ alkoxy, carbonyl optionally substituted by one substituent selected from C₃₋₈ cycloalkyl, thiophenyl, pyridinyl, furanyl and tetrahydrofuranyl, C₁₋₆ alkylsulfonyl, phenyl, pyrazinyl, benzo[d]isothiazol-3-yl, benzoisoxazolyl optionally substituted by one or more halogens, thiazolyl, piperidinyl, and phenoxy.

In some embodiments, PARP inhibitors of the present disclosure are selected from compounds represented by Formula III:

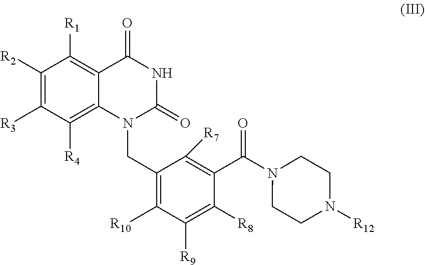

or pharmaceutically acceptable salts, solvates or prodrugs thereof, wherein:

R₁-R₄ are independently hydrogen, halo, optionally substituted amino, optionally substituted alkoxy, optionally substituted C₁₋₁₀ alkyl (such as haloalkyl, hydroxylalkyl, aminoalkyl, and carboxylalkyl), alkenyl, alkynyl, nitro, cyano, acylamido, hydroxy, thiol, acyloxy, azido, carboxy, ethylenedioxo, hydroxylamido or optionally substituted alkylthiol:

R₇-R₁₀ are independently hydrogen, halo, optionally substituted amino, alkoxy, C₁₋₁₀ alkyl, haloalkyl, aryl, heteroaryl, a carbocyclic group, a heterocyclic group, alkenyl, alkynyl, arylalkyl, arylalkenyl, arylalkynyl, heteroarylalkyl, heteroarylalkenyl, heteroarylalkynyl, carbocycloalkyl, heterocycloalkyl, hydroxyalkyl, hydroxyalkoxy, aminoalkyl, aminoalkoxy, carboxyalkyl, carboxyalkoxy, nitro, cyano, acylamido, aminocarbonyl, hydroxy, thiol, acyloxy, azido, carboxy, carbonylamido, alkylsulfonyl, aminosulfonyl, di-substituted alkylaminosulfonyl, alkylsulfiniyl, alkylthiol, or substituted carbonyl;

R₁₂ is an optionally substituted C₁₋₁₀ alkyl, haloalkyl, cycloalkyl, aryl, heteroaryl, carbocyclic group, heterocyclic group, alkenyl, alkynyl, acyl, arylalkyl, arylalkenyl, arylalkynyl, heteroarylalkyl, heteroarylalkenyl, heteroarylalkynyl, carbocycloalkyl, heterocycloalkyl, hydroxyalkyl, aminoalkyl, carboxyalkyl, alkylcarbonyl, cycloalkylcarbonyl, arylcarbonyl, heteroarylcarbonyl, heterocyclocarbonyl, aminocarbonyl, alkylsulfonyl, cycloalkylsulfonyl or aminosulfonyl.

In some embodiments of compounds of Formula III, R₁ and R₂ are independently selected from hydrogen, halo, C₁₋₆ alkyl and C₁₋₆ alkoxy; R₃ is H; R₄ is selected from H, halo, C₁₋₆ alkyl, C₁₋₆ alkoxy and hydroxy; R₇, R₈, R₉ and R₁₀ are independently hydrogen or halo; R₁₂ is an optionally substituted cycloalkyl, aryl, heteroaryl, carbocyclic group, heterocyclic group, arylalkyl, heteroarylalkyl, carbocycloalkyl, heterocycloalkyl, alkylcarbonyl, cycloalkylcarbonyl, arylcarbonyl, heteroarylcarbonyl or heterocyclocarbonyl. In some embodiments, R₁₂ is an optionally substituted C₃₋₈ cycloalkyl, pyridyl, pyrimidinyl, benzoyl, phenyl, piperidinyl, thienylcarbonyl, furanylcarbonyl, piperazinyl or thiazolyl.

In some embodiments of compounds of Formula III, R₁₂ is an optionally substituted cycloalkyl, aryl, heteroaryl, carbocyclic group, heterocyclic group, arylalkyl, heteroarylalkyl, carbocycloalkyl, heterocycloalkyl, alkylcarbonyl, cycloalkylcarbonyl, arylcarbonyl, heteroarylcarbonyl or heterocyclocarbonyl. In some embodiments, in compounds of Formula III, R₁ and R₂ are independently selected from hydrogen, fluoro, chloro, bromo and methyl; R₄ is hydrogen, fluoro, methoxy or hydroxy; R₇, R₈, R₉ and R₁₀ are independently hydrogen or halo, especially fluoro. In some embodiments, R₁₂ is an optionally substituted C₃₋₈ cycloalkyl, pyridyl, pyrimidinyl, benzoyl, phenyl, piperidinyl, thienylcarbonyl, furanylcarbonyl, piperazinyl or thiazolyl.

In some embodiments of compounds of Formula III, R₁ is halo, NH₂, C₁₋₆ alkyl, nitro or hydroxy, and R₂-R₄ are independently hydrogen, halo, NH₂, C₁₋₆ alkyl, nitro or hydroxy; or R₂ is halo, NH₂, C₁₋₆ alkyl, nitro or hydroxy, and R₁ and R₃-R₄ are independently hydrogen, halo, NH₂, C₁₋₆ alkyl, nitro or hydroxy; or R₃ is halo, NH₂, C₁₋₆ alkyl, nitro or hydroxyl, and R₁, R₂ and R₄ are independently hydrogen, halo, NH₂, C₁₋₆ alkyl, nitro or hydroxy; or R₄ is halo, NH₂, C₁₋₆ alkyl, nitro or hydroxyl, and R₁-R₃ are independently hydrogen, halo. NH₂, C₁₋₆ alkyl, nitro or hydroxyl; R₇-R₁₀ are independently hydrogen, halo, C₁₋₆ alkoxy or nitro; R₁₂ is pyridinyl, pyrimidinyl, C₃₋₈ cycloalkyl, C₁₋₆ alkyl optionally substituted by one C₃₋₈ cycloalkyl, benzoyl optionally substituted by one or more substituents selected from halo and C₁₋₆ alkoxy, carbonyl optionally substituted by one substituent selected from C₁₋₈ cycloalkyl, thiophenyl, pyridinyl, furanyl and tetrahydrofuranyl, C₁₋₆ alkylsulfonyl, phenyl, pyrazinyl, benzo[d]isothiazol-3-yl, benzoisoxazolyl optionally substituted by one or more halogens, and thiazolyl. In some embodiments, R₁ or R₂ is fluoro, chloro, bromo or methyl, R₃ is hydrogen, fluoro, methyl or methoxy; R₄ is hydrogen, fluoro, methyl, methoxy or hydroxy; R₇, R₈, R₉ or R₁₀ is hydrogen or fluoro. In some embodiments, in these embodiments, R₁₂ is C₃₋₈ cycloalkyl, phenyl, pyridyl, pyrimidinyl, or carbonyl optionally substituted by one substituent selected from C₃₋₈ cycloalkyl, thiophenyl, pyridyl, furanyl and tetrahydrofuranyl.

In some embodiments of compounds of Formula III, R₁ is halo or C₁₋₆ alkyl, R₂ is hydrogen, halo, —NH₂ or C₁₋₆ alkyl, R₃ is hydrogen, halo, C₁₋₆ alkoxy or C₁₋₆ alkyl, and R₄ is hydrogen, halo, hydroxy, C₁₋₆ alkoxy or C₁₋₆ alkyl; or R₁ is hydrogen, halo or C₁₋₆ alkyl, R₂ is halo, —NH₂ or C₁₋₆ alkyl, R₃ is hydrogen, halo, C₁₋₆ alkoxy or C₁₋₆ alkyl, and R₄ is hydrogen, halo, hydroxy, C₁₋₆ alkoxy or C₁₋₆ alkyl; or R₁ is hydrogen, halo or C₁₋₆ alkyl, R₂ is hydrogen, halo, —NH₂ or C₁₋₆ alkyl, R₃ is halo, C₁₋₆ alkoxy or C₁₋₆ alkyl, and R₄ is hydrogen, halo, hydroxy, C₁₋₆ alkoxy or C₁₋₆ alkyl; or R₁ is hydrogen, halo or C₁₋₆ alkyl, R₂ is hydrogen, halo, —NH₂ or C₁₋₆ alkyl, R₃ is hydrogen, halo, C₁₋₆ alkoxy or C₁₋₆ alkyl, and R₄ is halo, hydroxy, C₁₋₆ alkoxy or C₁₋₆ alkyl; R₇-R₁₀ are independently hydrogen, halogen, or C₁₋₆ alkyl; R₁₂ is C₃₋₈ cycloalkyl, C₃₋₄ cycloalkylcarbonyl, pyridyl, pyrimidinyl, benzoyl, phenyl, piperidinyl, thiophenylcarbonyl, furanyl or pyrazinyl, which is optionally substituted by 1, 2, 3, or 4 substituents selected from halo and C₁₋₆ alkyl. In some embodiments, R₁ is fluoro, chloro, bromo or methyl, R₂ is hydrogen, fluoro, chloro, bromo or methyl, R₃ is hydrogen, fluoro, chloro or methyl, R₄ is hydrogen, fluoro, chloro, methoxy or methyl, and R₇, R₈, R₉ and R₁₀ are independently hydrogen or fluoro; or R₂ is fluoro, chloro, bromo or methyl, R₁ is hydrogen, fluoro, chloro, bromo or methyl, R₃ is hydrogen, fluoro, chloro or methyl, R₄ is hydrogen, fluoro, methoxy or hydroxy, and R₇, R₈, R₉ and R₁₀ are independently hydrogen or fluoro; or R₄ is fluoro, methoxy or hydroxy, R₁ is hydrogen, fluoro, chloro, bromo or methyl, R₂ is hydrogen, fluoro, chloro, bromo or methyl, R₃ is hydrogen, fluoro, chloro or methyl, R₇, R₈, R₄ and R₁₀ are independently hydrogen or fluoro.

In some embodiments of compounds of Formula II, R₁ is halo, R₂ is hydrogen, halo or C₁₋₆ alkyl; R₃ is hydrogen, halo or C₁₋₆ alkyl; R₄ is hydrogen, halo or C₁₋₆ alkyl; R₇-R₁₀ are independently hydrogen or halo; R₁₂ is pyrimidinyl.

The definitions of groups herein such as alkyl, aryl, heteroaryl, heterocyclic group, amino, alkoxy, haloalkyl, alkenyl, alkynyl, amido, acyloxy, etc. and substituents on each group are described in PCT/CN2012/073362 (corresponding to U.S. Pat. No. 9,290,460).

PARP inhibitors include, without limitation:
1-(3-(4-(Pyridin-2-yl)piperazine-1-carbonyl)benzyl)quinazoline-2,4(1H,3H)-dione;
1-(3-(4-(Pyrimidin-2-yl)piperazine-1-carbonyl)benzyl)quinazoline-2,4(1H,3H)-dione;
1-(3-(4-Cyclohexylpiperazine-1-carbonyl)benzyl)quinazoline-2,4(1H,3-1)-dione;
1-(3-(4-([1,2,4]Triazolo[4,3-b]pyridazin-6-yl)piperazine-1-carbonyl)benzyl)quinazoline-2, 4(1H,3H)-dione;
1-(3-(4-Ethylpiperazine-1-carbonyl benzyl)quinazoline-2.4(1H,3H)-dione;
1-(3-(4-Benzoylpiperazine-1-carbonyl)benzyl)quinazoline-2,4(1H,3H)-dione;
1-(3-(4-(4-Fluorobenzoyl)piperazine-1-carbonyl)benzyl)quinazoline-2,4(1H,3H)-dione,
1-(3-(4-(4-Chlorobenzoyl)piperazine-1-carbonyl)benzyl)quinazoline-2,4(1H,3H)-dione;
1-(3-(4-(4-Bromobenzoyl)piperazine-1-carbonyl)benzyl)quinazoline-2,4(1H,3H)-dione;
1-(3-(4-(4-Methoxybenzoyl)piperazine-1-carbonyl)benzyl)quinazoline-2,4(1H,3H)-dione;
1-(3-(4-(Tetrahydro-2H-pyran-4-yl)carbonylpiperazine-1-carbonyl)benzyl)quinazoline-2,4 (1H,3H)-dione;
1-(3-(4-(Cyclopentylcarbonyl)piperazine-1-carbonyl)benzyl)quinazoline-2,4(1H,3H)-dione;
1-(3-(4-(Cyclopropylcarbonyl)piperazine-1-carbonyl)benzyl)quinazoline-2,4(1H,3H)-dione;
1-(3-(4-(Ethylsulfonyl)piperazine-1-carbonyl)benzyl)quinazoline-2,4(1H,3H)-dione;
1-(3-(4-Acetylpiperazine-1-carbonyl)benzyl)quinazoline-2,4(1H,3H)-dione;
1-(3-(4-Phenylpiperidine-1-carbonyl)benzyl)quinazoline-2,4(1H,3H)-dione;
1-(3-(4-Phenylpiperazine-1-carbonyl)benzyl)quinazoline-2,4(1H,3H)-dione;
1-(3-(4-(Pyrazin-2-yl)piperazine-1-carbonyl)benzyl)quinazoline-2,4(1H,3H)-dione;
1-(4-Fluoro-3-methoxycarbonylbenzyl)quinazoline-2,4(1H,3H)-dione;
1-(3-Carbonyl-4-fluorobenzyl)quinazoline-2,4(1H,3H)-dione;
1-(4-Fluoro-3-(4-(Cyclopentylcarbonyl)piperazine-1-carbonyl)benzyl)quinazoline-2,4(1H, 3H)-dione;
1-(4-Fluoro-3-(4-(pyridin-2-yl)piperazine-1-carbonyl)benzyl)quinazoline-2,4(1H,3H)-dione;
1-(4-Fluoro-3-(4-(pyrimidin-2-yl)piperazine-1-carbonyl)benzyl)quinazoline-2,4(1H,3H)-dione;
1-(3-(4-(Cyclohexylcarbonyl)piperazine-1-carbonyl)benzyl)quinazoline-2,4(1H,3H)-dione
1-(3-(4-(Benzo[d]isothiazol-3-yl)piperazine-1-carbonyl)benzyl)quinazoline-2,4(1H,3H)-dione;
1-(3-(4-(Piperidin-1-yl)piperidine-1-carbonyl)benzyl)quinazoline-2,4(1H,3H)-dione;
1-(3-(4-(Pyridin-4-yl)piperazine-1-carbonyl)benzyl)quinazoline-2,4(1H,3H)-dione;
1-(3-(4-(Cyclobutylcarbonyl)piperazine-1-carbonyl)benzyl)quinazoline-2,4(1H,3H)-dione;
1-(3-(4-(6-Fluorobenzo[d]isoxazol-3-yl)piperidine-1-carbonyl)benzyl)quinazoline-2,4(1H, 3H)-dione;
1-(3-(4-(Thiophene-2-carbonyl)piperazine-1-carbonyl)benzyl)quinazoline-2,4(1H,3H)-dione;
1-(3-(4-(Furan-2-carbonyl)piperazine-1-carbonyl)benzyl)quinazoline-2,4(1H,3H)-dione;
1-(3-(4-(Furan-3-carbonyl)piperazine-1-carbonyl)benzyl)quinazoline-2,4(1H,3H)-dione;
1-(3-(4-(Thiophene-3-carbonyl)piperazine-1-carbonyl)benzyl)quinazoline-2,4(1H,3H)-dione;
1-(3-(4-(Pyridine-3-carbonyl)piperazine-1-carbonyl)benzyl)quinazoline-2,4(1H,3H)-dione;
1-(3-(4-(Pyridine-2-carbonyl)piperazine-1-carbonyl)benzyl)quinazoline-2,4(1H,3H)-dione;
1-(3-(4-(Pyridine-4-carbonyl)piperazine-1-carbonyl)benzyl)quinazoline-2,4(1H,3H)-dione;
1-(3-(4-Phenoxypiperidine-1-carbonyl)benzyl)quinazoline-2,4(1H,3H)-dione,
1-(4-Fluoro-3-(4-(cyclopropylcarbonyl)piperazine-1-carbonyl)benzyl)quinazoline-2,4(1H, 3H)-dione;
1-(4-Fluoro-3-(4-(cyclohexylcarbonyl)piperazine-1-carbonyl)benzyl)quinazoline-2,4(1H,3H)-dione;
1-(4-Fluoro-3-(4-(pyrazin-2-yl)piperazine-1-carbonyl)benzyl)quinazoline-2,4(1H,3H)-dione;
1-(4-Fluoro-3-(4-(benzo[d]isothiazol-3-yl)piperazine-1-carbonyl)benzyl)quinazoline-2,4(1H,3H)-dione;
1-(4-Fluoro-3-(4-(cyclobutylcarbonyl)piperazine-1-carbonyl)benzyl)quinazoline-2,4(1H, 3H)-dione;
1-(4-Fluoro-3-(4-benzoylpiperazine-1-carbonyl)benzyl)quinazoline-2,4(1H,3H)-dione;
1-(4-Fluoro-3-(4-(thiophene-2-carbonyl)piperazine-1-carbonyl)benzyl)quinazoline-2,4(1H, 3H)-dione;
1-(4-Fluoro-3-(4-(furan-2-carbonyl)piperazine-1-carbonyl)benzyl)quinazoline-2,4(1H,3H)-dione;
1-(6-Fluoro-3-(4-(cyclopentylcarbonyl)piperazine-1-carbonyl)benzyl)quinazoline-2,4(1H, 3H)-dione;
1-(6-Fluoro-3-(4-(cyclohexylcarbonyl)piperazine-1-carbonyl)benzyl)quinazoline-2,4(1H, 3H)-dione,
1-(6-Fluoro-3-(4-(pyrimidin-2-yl)piperazine-1-carbonyl)benzyl)quinazoline-2,4(1H,3H)-dione;
1-(6-Fluoro-3-(4-(pyridin-2-yl)piperazine-1-carbonyl)benzyl)quinazoline-2,4(1H,3H)-dione;
1-(6-Fluoro-3-(4-(thiophene-2-carbonyl)piperazine-1-carbonyl)benzyl)quinazoline-2,4(1H, 3H)-dione;
1-(6-Fluoro-3-(4-(cyclobutylcarbonyl)piperazine-1-carbonyl)benzyl)quinazoline-2,4(1H, 3H)-dione;
1-(6-Fluoro-3-(4-(furan-2-carbonyl)piperazine-1-carbonyl)benzyl)quinazoline-2,4(1H,3H)-dione;
1-(6-Chloro-3-(4-(cyclopentylcarbonyl)piperazine-1-carbonyl)benzyl)quinazoline-2,4(1H, 3H)-dione;
1-(6-Chloro-3-(4-(pyrimidin-2-yl)piperazine-1-carbonyl)benzyl)quinazoline-2,4(1H,3H)-dione;
1-(6-Chloro-3-(4-(cyclohexylcarbonyl)piperazine-1-carbonyl)benzyl)quinazoline-2,4(1H, 3H)-dione;
1-(6-Chloro-3-(4-(pyridin-2-yl)piperazine-1-carbonyl)benzyl)quinazoline-2,4(1H,3H)-dione;
1-(2-Chloro-3-(4-(cyclopentylcarbonyl)piperazine-1-carbonyl)benzyl)quinazoline-2,4(1H, 3H)-dione;
1-(2-Chloro-3-(4-(pyrimidin-2-yl)piperazine-1-carbonyl)benzyl)quinazoline-2,4(1H,3H)-dione;
1-(2-Fluoro-3-(4-(pyrimidin-2-yl)piperazine-1-carbonyl)benzyl)quinazoline-2,4(1H,3H)-dione;
1-(2-Fluoro-3-(4-(cyclopentylcarbonyl)piperazine-1-carbonyl)benzyl)quinazoline-2,4(1H, 3H)-dione;
1-(5-Fluoro-3-(4-(pyrimidin-2-yl)piperazine-1-carbonyl)benzyl)quinazoline-2,4(1H,3H)-dione;
1-(5-Fluoro-3-(4-(cyclopentylcarbonyl)piperazine-1-carbonyl)benzyl)quinazoline-2,4(1H, 3H)-dione,
1-(4-Chloro-3-(4-(pyrimidin-2-yl)piperazine-1-carbonyl)benzyl)quinazoline-2,4(1H,3H)-dione;
1-(4-Chloro-3-(4-(cyclohexylcarbonyl)piperazine-1-carbonyl)benzyl)quinazoline-2,4(1H, 3H)-dione;
1-((2-(4-(Cyclopentylcarbonyl)piperazine-1-carbonyl)furan-5-yl)methyl)quinazoline-2, 4(1H,3H)-dione;
1-((2-(4-(Benzo[d]isothiazol-3-yl)piperazine-1-carbonyl)furan-5-yl)methyl)quinazoline-2, 4(1H,3H)-dione;
1-((2-(4-(Cyclopentylcarbonyl)piperazine-1-carbonyl)pyridin-6-yl)methyl)quinazoline-2,4 (1H,3H)-dione;
1-((2-(4-(Pyridin-2-yl)piperazine-1-carbonyl)pyridin-6-yl)methyl)quinazoline-2,4(1H,3H)-dione;
1-((2-(4-(Pyrimidin-2-yl)piperazine-1-carbonyl)pyridin-6-yl)methyl)quinazoline-2,4(1H, 3H)-dione;
1-(3-(4-(Thiazol-2-yl)piperazine-1-carbonyl)benzyl)quinazoline-2,4(1H,3H)-dione;
1-(3-(4-(Cyclohexylmethyl)piperazine-1-carbonyl)benzyl)quinazoline-2,4(1H,3H)-dione;
1-(3-(4-(Cyclopentylmethyl)piperazine-1-carbonyl)benzyl)quinazoline-2,4(1H,3H)-dione;
1-(3-(4-(Cyclohexylsulfonyl)piperazine-1-carbonyl)benzyl)quinazoline-2,4(1H,3H)-dione;
7-Fluoro-1-(3-(4-(cyclopentylcarbonyl)piperazine-1-carbonyl)benzyl)quinazoline-2,4(1H, 3H)-dione;
7-Fluoro-1-(3-(4-(pyrimidin-2-yl)piperazine-1-carbonyl)benzyl)quinazoline-2,4(1H,3H)-dione;
7-Methyl-1-(3-(4-(cyclopentylcarbonyl)piperazine-1-carbonyl)benzyl)quinazoline-2,4(1H, 3H)-dione;
7-Methyl-1-(3-(4-(pyrimidin-2-yl)piperazine-1-carbonyl)benzyl)quinazoline-2,4(1H,3H)-dione;
6-Methyl-1-(3-(4-(pyrimidin-2-yl)piperazine-1-carbonyl)benzyl)quinazoline-2,4(1H,3H)-dione;
6-Methyl-1-(3-(4-(cyclopentylcarbonyl)piperazine-1-carbonyl)benzyl)quinazoline-2,4(1H, 3H)-dione,
6-Methyl-1-(4-Fluoro-3-(4-(pyrimidin-2-yl)piperazine-1-carbonyl)benzyl)quinazoline-2,4 (1H,3H)-dione;
6-Methyl-1-(4-fluoro-3-(4-(cyclohexylcarbonyl)piperazine-1-carbonyl)benzyl)quinazoline-2,4(1H,3H)-dione;
6-Methyl-1-(4-fluoro-3-(4-(cyclopentylcarbonyl)piperazine-1-carbonyl)benzyl)quinazoline-2,4(1H,3H)-dione;
6-Bromo-1-(3-(4-(cyclohexylcarbonyl)piperazine-1-carbonyl benzyl)quinazoline-2,4(1H, 3H)-dione;
6-Bromo-1-(3-(4-(cyclopentylcarbonyl)piperazine-1-carbonyl)benzyl)quinazoline-2,4(1H, 3H)-dione,
6-Bromo-1-(3-(4-(pyrimidin-2-yl)piperazine-1-carbonyl)benzyl)quinazoline-2,4(1H,3H)-dione;
6-Nitro-1-(3-(4-(cyclopentylcarbonyl)piperazine-1-carbonyl)benzyl)quinazoline-2,4(1H, 3H)-dione;
6-Nitro-1-(3-(4-(pyrimidin-2-yl)piperazine-1-carbonyl)benzyl)quinazoline-2,4(1H,3H)-dione;
6-Nitro-1-(3-(4-(cyclohexylcarbonyl)piperazine-1-carbonyl)benzyl)quinazoline-2,4(1H,3H)-dione;
1-(4-(4-Benzoylpiperazine-1-carbonyl)benzyl)quinazoline-2,4(1H,3H)-dione;
1-(4-(4-(Pyridin-2-yl)piperazine-1-carbonyl)benzyl)quinazoline-2,4(1H,3H)-dione,
1-(4-(4-(4-Methoxybenzoyl)piperazine-1-carbonyl)benzyl)quinazoline-2,4(1H,3H)-dione;
1-(4-(4-(4-Fluorobenzoyl)piperazine-1-carbonyl)benzyl)quinazoline-2,4(1H,3H)-dione,
1-(4-(4-(Pyrimidin-2-yl)piperazine-1-carbonyl)benzyl)quinazoline-2,4(1H,3H)-dione;
1-(4-(4-(4-Bromobenzoyl)piperazine-1-carbonyl)benzyl)quinazoline-2,4(1H,3H)-dione;
1-(4-(4-(Cyclopentylcarbonyl)piperazine-1-carbonyl)benzyl)quinazoline-2,4(1H,3H)-dione;
5-Chloro-1-(3-(4-(pyrimidin-2-yl)piperazine-1-carbonyl)benzyl)quinazoline-2,4(1H,3H)-dione;
5-Chloro-1-(3-(4-(cyclopentylcarbonyl)piperazine-1-carbonyl)benzyl)quinazoline-2,4(1H, 3H)-dione;
5-Chloro-1-(3-(4-(cyclohexylcarbonyl)piperazine-1-carbonyl)benzyl)quinazoline-2,4(1H, 3H)-dione;
5-Chloro-1-(4-fluoro-3-(4-(pyrimidin-2-yl)piperazine-1-carbonyl)benzyl)quinazoline-2,4(1H,3H)-dione;
5-Chloro-1-(4-fluoro-3-(4-(cyclopentylcarbonyl)piperazine-1-carbonyl)benzyl)quinazoline-2,4(1H,3H)-dione;
5-Chloro-1-(4-fluoro-3-(4-(cyclohexylcarbonyl)piperazine-1-carbonyl)benzyl)quinazoline-2,4(1H,3H)-dione;
5-Chloro-1-(4-fluoro-3-(4-(pyridin-2-yl)piperazine-1-carbonyl)benzyl)quinazoline-2,4(1H, 3H)-dione,
5-Chloro-1-(4-fluoro-3-(4-(thiophene-2-carbonyl)piperazine-1-carbonyl)benzyl) quinazoline-2,4(1H,3H)-dione;
5-Chloro-1-(6-fluoro-3-(4-(cyclohexylcarbonyl)piperazine-1-carbonyl)benzyl)quinazoline-2,4(1H,3H)-dione;
5-Chloro-1-(6-fluoro-3-(4-(cyclopentylcarbonyl)piperazine-1-carbonyl)benzyl)quinazoline-2,4(1H,3H)-dione;
5-Chloro-1-(6-fluoro-3-(4-(pyrimidin-2-yl)piperazine-1-carbonyl)benzyl)quinazoline-2,4(1H,3H)-dione;
6-Chloro-1-(3-(4-(pyrimidin-2-yl)piperazine-1-carbonyl)benzyl)quinazoline-2,4(1H,3H)-dione;
6-Chloro-1-(3-(4-(cyclopentylcarbonyl)piperazine-1-carbonyl)benzyl)quinazoline-2,4(1H, 3H)-dione;
6-Chloro-1-(3-(4-(cyclohexylcarbonyl)piperazine-1-carbonyl)benzyl)quinazoline-2,4(1H, 3H)-dione;
6-Chloro-1-(3-(4-(pyridin-2-yl)piperazine-1-carbonyl)benzyl)quinazoline-2,4(1H,3H)-dione;
6-Chloro-1-(3-(4-(thiophene-2-carbonyl)piperazine-1-carbonyl)benzyl)quinazoline-2,4(1H,3H)-dione;
6-Chloro-1-(3-(4-(cyclobutylcarbonyl)piperazine-1-carbonyl)benzyl)quinazoline-2,4(1H,3H)-dione;
6-Chloro-1-(4-fluoro-3-(4-(pyrimidin-2-yl)piperazine-1-carbonyl)benzyl)quinazoline-2,4(1H,3H)-dione,
6-Chloro-1-(3-(4-(cyclopentylcarbonyl)piperazine-1-carbonyl)-4-fluorobenzyl)quinazoline-2,4(1H,3H)-dione;
6-Chloro-1-(3-(4-(cyclohexylcarbonyl)piperazine-1-carbonyl)-4-fluorobenzyl)quinazoline-2,4(1H,3H)-dione;
6-Chloro-1-(4-fluoro-3-(4-(pyridin-2-yl)piperazine-1-carbonyl)benzyl)quinazoline-2,4(1H, 3H)-dione;
6-Chloro-1-(4-fluoro-3-(4-(thiophene-2-carbonyl)piperazine-1-carbonyl)benzyl) quinazoline-2,4(1H,3H)-dione;
6-Chloro-1-(4-fluoro-3-(4-(cyclopropylcarbonyl)piperazine-1-carbonyl)benzyl)quinazoline)-2,4(1H,3H)-dione;
6-Chloro-1-(6-fluoro-3-(4-(pyrimidin-2-yl)piperazine-1-carbonyl)benzyl)quinazoline-2,4(1H,3H)-dione;
6-Chloro-1-(6-fluoro-3-(4-(cyclopentylcarbonyl)piperazine-1-carbonyl)benzyl)quinazoline-2,4(1H,3H)-dione;
6-Chloro-1-(6-fluoro-3-(4-(cyclohexylcarbonyl)piperazine-1-carbonyl)benzyl)quinazoline-2,4(1H,3H)-dione;
6-Chloro-1-(6-fluoro-3-(4-(thiophene-2-carbonyl)piperazine-1-carbonyl)benzyl) quinazoline-2,4(1H,3H)-dione;
5-Fluoro-1-(3-(4-(pyrimidin-2-yl)piperazine-1-carbonyl)benzyl)quinazoline-2,4(1H,3H)-dione;
5-Fluoro-1-(3-(4-(Cyclopentylcarbonyl)piperazine-1-carbonyl)benzyl)quinazoline-2,4(1H, 3H)-dione;
5-Fluoro-1-(3-(4-(Cyclohexylcarbonyl)piperazine-1-carbonyl)benzyl)quinazoline-2,4(1H, 3H)-dione;
5-Fluoro-1-(3-(4-(pyridin-2-yl)piperazine-1-carbonyl)benzyl)quinazoline-2,4(1H,3H)-dione;
5-Fluoro-(1-(3-(4-(thiophene-2-carbonyl)piperazine-1-carbonyl)benzyl)quinazoline)-2,4 (1H,3H)-dione;
5-Fluoro-1-(4-fluoro-3-(4-(pyrimidin-2-yl)piperazine-1-carbonyl)benzyl)quinazoline-2,4 (1H,3H)-dione;
5-Fluoro-1-(4-fluoro-3-(4-(cyclopentylcarbonyl)piperazine-1-carbonyl)benzyl)quinazoline-2,4(1H,3H)-dione;
5-Fluoro-1-(4-fluoro-3-(4-(cyclohexylcarbonyl)piperazine-1-carbonyl)benzyl)quinazoline-2,4(1H,3H)-dione;
5-Fluoro-1-(4-fluoro-3-(4-(pyridin-2-yl)piperazine-1-carbonyl)benzyl)quinazoline-2,4(1H, 3H)-dione;
5-Fluoro-1-(4-fluoro-3-(4-(thiophene-2-carbonyl)piperazine-1-carbonyl)benzyl) quinazoline-2,4(1H,3H)-dione;
5-Fluoro-1-(4-fluoro-3-(4-Benzoylpiperazine-1-carbonyl)benzyl)quinazoline-2,4(1H,3H)-dione;
8-Fluoro-1-(3-(4-(cyclohexylcarbonyl)piperazine-1-carbonyl)benzyl)quinazoline-2,4(1H, 3H)-dione;
6-Fluoro-1-(3-(4-(pyrimidin-2-yl)piperazine-1-carbonyl)benzyl)quinazoline-2,4(1H,3H)-dione;
6-Fluoro-1-(3-(4-(pyridin-2-yl)piperazine-1-carbonyl)benzyl)quinazoline-2,4(1H,3H)-dione;
6-Fluoro-(1-(3-(4-(thiophene-2-carbonyl)piperazine-1-carbonyl)benzyl)quinazoline)-2,4 (1H,3H)-dione;
6-Fluoro-1-(3-(4-(cyclohexylcarbonyl)piperazine-1-carbonyl)benzyl)quinazoline-2,4(1H, 3H)-dione;
6-Fluoro-1-(3-(4-(cyclopentylcarbonyl)piperazine-1-carbonyl)benzyl)quinazoline-2,4(1H, 3H)-dione;
6-Fluoro-1-(4-fluoro-3-(4-(pyrimidin-2-yl)piperazine-1-carbonyl)benzyl)quinazoline-2,4 (1H,3H)-dione;
6-Fluoro-1-(4-fluoro-3-(4-(pyridin-2-yl)piperazine-1-carbonyl)benzyl)quinazoline-2,4(1H, 3H)-dione;
6-Fluoro-1-(4-fluoro-3-(4-(thiophene-2-carbonyl)piperazine-1-carbonyl)benzyl) quinazoline-2,4(1H,3H)-dione;
6-Fluoro-1-(4-fluoro-3-(4-(cyclohexylcarbonyl)piperazine-1-carbonyl)benzyl)quinazoline-2,4(1H,3H)-dione;
6-Fluoro-1-(4-fluoro-3-(4-(cyclopentylcarbonyl)piperazine-1-carbonyl)benzyl)quinazoline-2,4(1H,3H)-dione;
6-Fluoro-1-(6-fluoro-3-(4-(pyrimidin-2-yl)piperazine-1-carbonyl)benzyl)quinazoline-2,4 (1H,3H)-dione;
6-Fluoro-1-(6-fluoro-3-(4-(pyridin-2-yl)piperazine-1-carbonyl)benzyl)quinazoline-2,4(1H, 3H)-dione;
6-Fluoro-1-(6-fluoro-3-(4-(thiophene-2-carbonyl)piperazine-1-carbonyl)benzyl) quinazoline-2,4(1H,3H)-dione;
6-Fluoro-1-(6-fluoro-3-(4-(cyclohexylcarbonyl)piperazine-1-carbonyl)benzyl)quinazoline-2,4(1H,3H)-dione,
6-Fluoro-1-(6-fluoro-3-(4-(cyclopentylcarbonyl)piperazine-1-carbonyl)benzyl)quinazoline-2,4(1H,3H)-dione;
5-Fluoro-1-(6-fluoro-3-(4-(pyrimidin-2-yl)piperazine-1-carbonyl)benzyl)quinazoline-2,4 (1H,3H)-dione;
5-Fluoro-1-(6-fluoro-3-(4-(pyridin-2-yl)piperazine-1-carbonyl)benzyl)quinazoline-2,4(1H, 3H)-dione;
5-Fluoro-1-(6-fluoro-3-(4-(thiophene-2-carbonyl)piperazine-1-carbonyl)benzyl) quinazoline-2,4(1H,3H)-dione;
5-Fluoro-1-(6-fluoro-3-(4-(cyclohexylcarbonyl)piperazine-1-carbonyl)benzyl)quinazoline-2,4(1H,3H)-dione;
5-Fluoro-1-(6-fluoro-3-(4-(cyclopentylcarbonyl)piperazine-1-carbonyl)benzyl)quinazoline-2,4(1H,3H)-dione;
5-Methyl-1-(3-(4-(pyrimidin-2-yl)piperazine-1-carbonyl)benzyl)quinazoline-2,4(1H,3H)-dione;
5-Methyl-1-(3-(4-(cyclopentylcarbonyl)piperazine-1-carbonyl)benzyl)quinazoline-2,4(1H, 3H)-dione;
7-Chloro-1-(3-(4-(pyrimidin-2-yl)piperazine-1-carbonyl)benzyl)quinazoline-2,4(1H,3H)-dione;
7-Chloro-1-(3-(4-(cyclopentylcarbonyl)piperazine-1-carbonyl)benzyl)quinazoline-2,4(1H, 3H)-dione;
5-Fluoro-1-(4-fluoro-3-(4-(cyclopropylcarbonyl)piperazine-1-carbonyl)benzyl)quinazoline)-2,4(1H,3H)-dione;
6-Chloro-(4-fluoro-1-(3-(4-benzoylpiperazine-1-carbonyl)benzyl)quinazoline)-2,4(1H,3H)-dione;
5-Fluoro-1-(3-(4-(cyclobutylcarbonyl)piperazine-1-carbonyl)benzyl)quinazoline-2,4(1H, 3H)-dione;
5-fluoro-1-(4-fluoro-3-(4-(cyclobutylcarbonyl)piperazine-1-carbonyl)benzyl)quinazoline-2,4(1H,3H)-dione,
6-Chloro-1-(6-fluoro-3-(4-(cyclobutylcarbonyl)piperazine-1-carbonyl)benzyl)quinazoline-2,4(1H,3H)-dione,
5-Chloro-1-(4-fluoro-3-(4-(cyclobutylcarbonyl)piperazine-1-carbonyl)benzyl)quinazoline-2,4(1H,3H)-dione;
6-Chloro-1-(4-fluoro-3-(4-(cyclobutylcarbonyl)piperazine-1-carbonyl)benzyl)quinazoline-2,4(1H,3H)-dione;
5-Chloro-1-(6-fluoro-3-(4-(cyclobutylcarbonyl)piperazine-1-carbonyl)benzyl)quinazoline-2,4(1H,3H)-dione;
5-Chloro-1-(6-fluoro-3-(4-(thiophene-2-carbonyl)piperazine-1-carbonyl)benzyl) quinazoline-2,4(1H,3H)-dione;
5-Fluoro-1-(4-fluoro-3-(4-(furan-2-carbonyl)piperazine-1-carbonyl)benzyl)quinazoline-2,4 (1H,3H)-dione;
5-Chloro-1-(6-fluoro-3-(4-(pyrindin-2-yl)piperazine-1-carbonyl)benzyl)quinazoline-2,4(1H, 3H)-dione;
6-Chloro-1-(6-fluoro-3-(4-(pyridin-2-yl)piperazine-1-carbonyl)benzyl)quinazoline-2,4(1H, 3H)-dione;
6-Chloro-1-(5-fluoro-3-(4-(pyrimidin-2-yl)piperazine-1-carbonyl)benzyl)quinazoline-2,4(1H,3H)-dione;
5-Fluoro-1-(5-fluoro-3-(4-(pyrimidin-2-yl)piperazine-1-carbonyl)benzyl)quinazoline-2,4 (1H,3H)-dione;
1-(3-(4-Cyclopentylpiperazine-1-carbonyl)benzyl)quinazoline-2,4(1H,3H)-dione;
6-Fluoro-1-(4-fluoro-3-(4-(cyclopropylcarbonyl)piperazine-1-carbonyl)benzyl)quinazoline-2,4(1H,3H)-dione;
6-Fluoro-1-(3-(4-(cyclobutylcarbonyl)piperazine-1-carbonyl)benzyl)quinazoline-2,4(1H, 3H)-dione;
6-Fluoro-1-(4-fluoro-3-(4-(cyclobutylcarbonyl)piperazine-1-carbonyl)benzyl)quinazoline-2,4(1H,3H)-dione;
6-Fluoro-1-(4-fluoro-3-(4-benzoylpiperazine-1-carbonyl)benzyl)quinazoline-2,4(1H,3H)-dione;
5-Fluoro-1-(6-fluoro-3-(4-(cyclobutylcarbonyl)piperazine-1-carbonyl)benzyl)quinazoline-2,4(1H,3H)-dione;
6-Fluoro-1-(6-fluoro-3-(4-(cyclobutylcarbonyl)piperazine-1-carbonyl)benzyl)quinazoline-2,4(1H,3H)-dione;
5-Fluoro-1-(6-fluoro-3-(4-(furan-2-carbonyl)piperazine-1-carbonyl)benzyl)quinazoline-2,4 (1H,3H)-dione;
6-Fluoro-1-(4-fluoro-3-(4-(furan-2-carbonyl)piperazine-1-carbonyl)benzyl)quinazoline-2,4 (1H,3H)-dione;
6-Fluoro-1-(5-fluoro-3-(4-(pyrimidin-2-yl)piperazine-1-carbonyl)benzyl)quinazoline-2,4 (1H,3H)-dione;
6-Fluoro-1-(6-chloro-3-(4-(pyrimidin-2-yl)piperazine-1-carbonyl)benzyl)quinazoline-2,4(1H,3H)-dione;
6-Fluoro-1-(4-fluoro-3-(4-(thiazol-2-yl)piperazine-1-carbonyl)benzyl)quinazoline-2,4(1H, 3H)-dione;
5-Fluoro-1-(4-fluoro-3-(4-(thiazol-2-yl)piperazine-1-carbonyl)benzyl)quinazoline-2,4(1H, 3H)-dione;
1-(4-Fluoro-3-(4-(thiazol-2-yl)piperazine-1-carbonyl)benzyl)quinazoline-2,4(1H,3H)-dione;
1-(3-(4-(Cyclohexylcarbamoyl)piperazine-1-carbonyl)benzyl)quinazoline-2,4(1H,3H)-dione;
1-(4-Fluoro-3-(4-(pyrimidin-2-yl)piperazine-1-carbonyl)benzyl)quinazoline-2,4(1H,3H)-dione hydrochloride;
5-Fluoro-1-(4-fluoro-3-(4-(pyrimidin-2-yl)piperazine-1-carbonyl)benzyl)quinazoline-2,4 (1H,3H)-dione hydrochloride;
1-(3-(4-(Pyrimidin-2-yl)piperazine-1-carbonyl)benzyl)quinazoline-2,4(1H,3H)-dione hydrochloride;
1-(3-((4-(Pyridin-2-yl)piperazin-1-yl)methyl)benzyl)quinazoline-2,4(H,31-1)-dione;
1-(3-(Naphthalen-2-yl)acetamidobenzyl)quinazoline-2,4(1H,3H)-dione;
1-(3-(3,4-Dimethoxyphenyl)acetamidobenzyl)quinazoline-2,4(1H,3H)-dione;
1-(3-([1,2,4]Triazolo[4,3-a]pyridine-6-carbonyl)benzyl)quinazoline-2,4(1H,3H)-dione;
6-Fluoro-1-((2-(4-(pyrimidin-2-yl)piperazine-1-carbonyl)pyridin-6-yl)methyl)quinazoline-2,4(1H,3H)-dione;
5-Fluoro-1-(4-fluoro-3-(4-methoxybenzylcarbamoyl)benzyl)quinazoline-2,4(1H,3H)-dione
5-Fluoro-1-(4-fluoro-3-(3-chlorobenzylcarbamoyl)benzyl)quinazoline-2,4(1H,3H)-dione;
5-Fluoro-1-(4-fluoro-3-(benzylcarbamoyl)benzyl)quinazoline-2,4(1H,3H)-dione;
5-Methoxy-1-(3-(4-(pyrimidin-2-yl)piperazine-1-carbonyl)benzyl)quinazoline-2,4(1H,3H)-dione;
6-Methoxy-1-(3-(4-(pyrimidin-2-yl)piperazine-1-carbonyl)benzyl)quinazoline-2,4(1H,3H)-dione;
5-Fluoro-1-(4-fluoro-3-(5-bromopyrimidin-2-aminocarbamoyl)benzyl)quinazoline-2,4(1H, 3H)-dione;
7-Trifluoromethyl-1-(3-(4-(Pyrimidin-2-yl)piperazine-1-carbonyl)benzyl)quinazoline-2,4(1H,3H)-dione;
6,7-Ethylenedioxo-1-(3-(4-(pyrimidin-2-yl)piperazine-1-carbonyl)benzyl)quinazoline-2,4(1H,3H)-dione;
5-Fluoro-1-(6-methoxy-3-(4-(pyrimidin-2-yl)piperazine-1-carbonyl)benzyl)quinazoline-2,4(1H,3H)-dione;
7-Methoxy-1-(3-(4-(pyrimidin-2-yl)piperazine-1-carbonyl)benzyl)quinazoline-2,4(1H,3H)-dione;
5-Fluoro-1-(4-fluoro-3-(4-(tetrahydrofuran-2-carbonyl)piperazine-1-carbonyl)benzyl) quinazoline-2,4(1H,3H)-dione;
5-Fluoro-1-(4-nitro-3-(4-(pyrimidin-2-yl)piperazine-1-carbonyl)benzyl)quinazoline-2,4 (1H,3H)-dione;
5-Fluoro-1-(4-fluoro-3-(4-cyclohexylpiperazine-1-carbonyl)benzyl)quinazoline-2,4(1H,3H)-dione;
5-Fluoro-1-(4-fluoro-3-(4-phenylpiperazine-1-carbonyl)benzyl)quinazoline-2,4(1H,3H)-dione;
5-Fluoro-1-(4-fluoro-3-(4-phenylpiperidine-1-carbonyl)benzyl)quinazoline-2,4(1H,3H)-dione;
5-Fluoro-1-(4-bromo-3-(4-(pyrimidin-2-yl)piperazine-1-carbonyl)benzyl)quinazoline-2,4(1H,3H)-dione;
6,7-Methylenedioxo-1-(3-(4-(pyrimidin-2-yl)piperazine-1-carbonyl)benzyl)quinazoline-2,4(1H,3H)-dione;
5-Fluoro-1-(4-fluoro-3-(4-(cyclohexylmethyl)piperazine-1-carbonyl)benzyl)quinazoline-2,4(1H,3H)-dione;
8-Fluoro-1-(3-(4-(pyrimidin-2-yl)piperazine-1-carbonyl)benzyl)quinazoline-2,4(1H,3H)-dione;
6-Amino-1-(3-(4-(pyrimidin-2-yl)piperazine-1-carbonyl)benzyl)quinazoline-2,4(1H,3H)-dione;
1-(2-(4-(Pyrimidin-2-yl)piperazine-1-carbonyl)benzyl)quinazoline-2,4(1H,3H)-dione;
8-Chloro-1-(3-(4-(pyrimidin-2-yl)piperazine-1-carbonyl)benzyl)quinazoline-2,4(1H,3H)-dione;
8-Methyl-1-(3-(4-(pyrimidin-2-yl)piperazine-1-carbonyl)benzyl)quinazoline-2,4(1H,3H)-dione;
8-Methoxy-1-(3-(4-(pyrimidin-2-yl)piperazine-1-carbonyl)benzyl)quinazoline-2,4(1H,3H)-dione;
8-Hydroxy-1-(3-(4-(pyrimidin-2-yl)piperazine-1-carbonyl)benzyl)quinazoline-2,4(1H,3H)-dione;

or pharmaceutically acceptable salts, solvates or prodrugs thereof.

Particular PARP inhibitors are 5-fluoro-1-(4-fluoro-3-(4-(pyrimidin-2-yl)piperazine-1-carbonyl)benzyl)quinazoline-2,4(1H,3H)-dione (also referred to herein as “IMP4297” and “senaparib”) and its pharmaceutically acceptable salts, solvates or prodrugs.

In the present disclosure, examples of the pharmaceutically acceptable salts include salts of inorganic and organic acid, such as hydrochloride, hydrobromide, phosphate, sulphate, citrate, lactate, tartrate, maleate, fumarate, mandelate and oxalate; and salts of inorganic and organic base formed with bases such as sodium hydroxy, tris(hydroxymethyl)aminomethane (TRIS, tromethamine) and N-methyl-glucamine.

In the present disclosure, examples of prodrugs of compounds include the simple esters of carboxylic acid-containing compounds (e.g., those obtained by condensation with a C₁₋₄ alcohol according to methods known in the art); esters of hydroxyl-containing compounds (e.g., those obtained by condensation with a C₁₋₄ carboxylic acid, C₂₋₆ diacid or anhydride thereof such as succinic anhydride and fumaric anhydride, according to methods known in the art); imines of amino-containing compounds (e.g., those obtained by condensation with a C₁₋₄ aldehyde or ketone according to methods known in the art), carbamate of amino-containing compounds, such as those described by Leu, et al. (J. Med. Chem., 42:3623-3628 (1999)) and Greenwald, et al. (J. Med. Chem., 42.3657-3667 (1999)), and acetals and ketals of alcohol-containing compounds (e.g., those obtained by condensation with chloromethyl methyl ether or chloromethyl ethyl ether according to methods known in the art).

Solvates of the PARP inhibitors of the present disclosure, including without limitation, hydrates, such as dihydrate, may be used.

DNA Damaging Anti-Cancer Drugs

In the present disclosure, the DNA damaging anti-cancer drug is in some embodiments an alkylating agent anti-cancer drug. Examples of the alkylating agent anti-cancer drug includes but is not limited to, nitrogen mustard N-oxide, cyclophosphamide, ifosfamide, thiotepa, ranimustine, nimustine, Temozolomide (TMZ), altretamine, apaziquone, brostallicin, bendamustine, carmustine, estramustine, fotemustine, glufosfamide, mafosfamide, bendamustine, and dibromidulcitol; and alkylated compounds with platinum coordination, including without limitation; cisplatin, carboplatin, eptaplatin, lobaplatin, nedaplatin, oxaliplatin and satraplatin.

In some embodiments, the alkylating agent anti-cancer drug is an imidazotetrazine alkylating agent with anti-tumor activity, more specifically, TMZ. TMZ can degrade in vivo spontaneously and quickly to produce an active metabolite MTIC, thereby producing anti-tumor effects.

It is known that TMZ can be used in the treatment of gliomas (such as glioblastoma multifonne, anaplastic astrocytoma), melanoma and lymphoma, breast cancer, lung cancer (including non-small cell lung cancer), refractory pituitary adenoma, gastric cancer, etc.

Cancers

The cancers that can be treated with the method or compositions or pharmaceutical preparations of the present disclosure are various cancers that can be treated with PARP inhibitors and DNA damaging anti-cancer drugs such as TMZ or can be treated with combination of PARP inhibitors and DNA damaging anti-cancer drugs such as TMZ. These cancers include but are not limited to liver cancer, melanoma (malignant melanoma), Hodgkin's disease, non-Hodgkin's lymphoma, acute lymphocytic leukemia, chronic lymphocytic leukemia, multiple myeloma, neuroblastoma, breast cancer, ovarian cancer, small cell lung cancer, non-small cell lung cancer, Wilms tumor, cervical cancer, testicular cancer, soft tissue sarcoma, primary macroglobulinemia, bladder cancer, primary brain cancer, gastric cancer, colon cancer, malignant pancreatic islet tumor, malignant carcinoid cancer, choriocarcinoma, mycosis fungoides, head and neck cancer, osteogenic sarcoma, pancreatic cancer, acute myeloid leukemia, hairy cell leukemia, rhabdomyosarcoma, Kaposi's sarcoma, urogenital tumors, thyroid cancer, esophageal cancer, malignant hypercalcemia, cervical hyperplasia, renal cell carcinoma, endometrial cancer, polycythemia vera, idiopathic thrombocythemia, adrenocortical carcinoma, skin cancer, prostate cancer, glioma (such as glioblastoma multiforme, anaplastic astrocytoma) and refractory pituitary adenoma, etc.

In some embodiments, the cancers that can be treated with the method or compositions or pharmaceutical preparations of the present disclosure especially include those being treated with TMZ, which include gliomas (such as glioblastoma multiforme, anaplastic astrocytoma), melanoma and lymphoma, breast cancer, lung cancer (including non-small cell lung cancer), refractory pituitary adenoma, gastric cancer, etc.

In some embodiments, the cancers to be treated with the method or compositions or pharmaceutical preparations of the present disclosure include but are not limited to pancreatic cancer, endometrial cancer, ovarian cancer, mesothelioma cancer, small-cell lung cancer, rectal cancer, and peripheral nerve sheath cancer.

Treatment Method

The treatment method of the present disclosure includes sequentially or simultaneously administering to a subject in need thereof an effective dose of a PARP inhibitor with trapping activity of the present disclosure and a non-toxic low dose of a DNA damaging anti-cancer drug described herein, such as TMZ. When administered sequentially, there is no special restriction on the order of administration of the PARP inhibitor and the DNA damaging anti-cancer drug. The PARP inhibitor can be administered first, and then the DNA damaging anti-cancer drug can be administered after a period of time, or the DNA damaging anti-cancer drug can be administered first, and then the PARP inhibitor can be administered after a period of time. Simultaneous administration includes taking or administering the two drugs at the same time, or administering one drug immediately after the other drug is administered, or administering the compound preparation of the present disclosure.

In the present disclosure, an effective dose of a PARP inhibitor refers to a dose that can achieve its intended purpose, and the intended purpose includes, but is not limited to, inhibiting tumor growth and/or killing cancer cells by acting as an anti-cancer drug, and strengthening the anti-cancer efficacy of a DNA damaging anti-cancer drug such as TMZ by acting as a trapping agent to trap the DNA lesions generated by TMZ, forming DNA-PARP complex and killing cancer cells effectively. While individual needs vary, determination of the effective dose or optimal dose of the PARP inhibitor used in the present disclosure is within the person skilled in the art. In general, the PARP inhibitor of the disclosure may be administered to mammals orally at a dose of about 0.0025 to 50 mg/kg of body weight per day. In some embodiments, the PARP inhibitor is administered to mammals orally at a dose of about 0.01 to 20 mg/kg of body weight per day. In some embodiments, the clinically effective daily dose of the PARP inhibitor IMP4297 of the present disclosure when being used alone is 20-120 mg. In some embodiments, the clinically effective daily dose of IMP4297 is 20-100 mg. In some embodiments, the clinically effective daily dose of IMP4297 is 40-80 mg, such as 40 mg, 60 mg, and 80 mg. In some embodiments, the dosing schedule of IMP4297 is QD continuously for days 1-28 of a 28-day cycle.

A “low dose” of a DNA damaging anti-cancer drug such as TMZ described herein refers to a daily dose that is about 1/12th to about ⅕th of the daily dose of known DNA damaging anti-cancer drugs when used alone or in combination with other drugs. It is known that the daily dose of current TMZ in combination with radiotherapy for newly diagnosed glioblastoma multiforme is 75 mg/m² (equivalent to 121.5 mg), and the lowest recommended dose of TMZ in monotherapy is 100 mg/m² (equivalent to 162 mg). Therefore, in the present disclosure, the “low dose” of TMZ is in the range of about 10 mg to about 35 mg (daily dose). In some embodiments, the “low dose” (daily dose) of the DNA damaging anti-cancer drug TMZ of the present disclosure is 10-30 mg. In some embodiments, the daily dose of TMZ is 20-30 mg. In some embodiments, the daily dose of TMZ is 20 mg. In some embodiments, the daily dose of TMZ is 10 mg. In some embodiments, the dosing schedule of TMZ is once a day continuously for days 1-21 of a 28-day cycle. In some embodiments, the low dose is a non-toxic dose.

In some embodiments, the daily dose of IMP4297 is 40 mg, and the daily dose of TMZ is 20 mg. In some embodiments, the daily dose of IMP4297 is 60 mg, and the daily dose of TMZ is 20 mg. In some embodiments, the daily dose of IMP4297 is 80 mg, and the daily dose of TMZ is 20 mg. In some embodiments, the daily dose of IMP4297 is 80 mg, and the daily dose of TMZ is 30 mg. In some embodiments, the daily dose of IMP4297 is 40 mg, and the daily dose of TMZ is 10 mg. In some embodiments, the daily dose of IMP4297 is 20 mg, and the daily dose of TMZ is 10 mg.

In some embodiments of the method for treating cancer of the present disclosure, the method comprises orally administering a subject in need thereof. IMP4297 in a daily dose of 20-120 mg, such as 20-100 mg, such as 40 mg, 60 mg, 80 mg or 100 mg, for 28 days, and TMZ in a daily dose of 10-30 mg, such as 10 mg, 20 mg or 30 mg, for 21 days, with administration of 28 days as one cycle. In some embodiments, TMZ is administered at day 1 to day 21 of the 28-day cycle.

Kits and Compound Preparations

The kit of the present disclosure contains one or more pharmaceutical preparations of PARP inhibitor described herein and one or more pharmaceutical preparations of DNA damaging anti-cancer drug described herein. In some embodiments, the kit of the present disclosure contains one or more pharmaceutical preparations of IMP4297 and one or more pharmaceutical preparations of TMZ in some embodiments, the kit of the present disclosure contains the pharmaceutical preparations of PARP inhibitor and the pharmaceutical preparations of DNA damaging anti-cancer drug in an amount sufficient to be administered in any of the dosing schecules as described herein.

The PARP inhibitor and the DNA damaging anti-cancer drug such as TMZ used in the method or kit of the present disclosure can be formulated into separate pharmaceutical preparations for sequential or simultaneous administration. The pharmaceutical preparation of the PARP inhibitor should be able to meet the dosage requirement on the effective daily dose of the PARP inhibitor when used alone. In some embodiments, the pharmaceutical preparation of IMP4297 should be able to meet the dosage requirement of 20-120 mg/day, and the pharmaceutical preparation of TMZ should meet the dosage requirement of 10-30 mg/day. In other words, the pharmaceutical preparations can be formulated into multiple doses (such as 2 or more capsules or tablets), but the total amount of the PARP inhibitor or TMZ in all their respective pharmaceutical preparations should meet the above-mentioned daily dosage requirements respectively.

The kit of the present disclosure is able to provide IMP4297 in a daily dose of 40 mg and TMZ in a daily dose of 20 mg, or IMP4297 in a daily dose of 60 mg and TMZ in a daily dose of 20 mg, or IMP4297 in a daily dose of 80 mg and TMZ in a daily dose of 20 mg, or IMP4297 in a daily dose of 80 mg and TMZ in a daily dose of 30 mg, or IMP4297 in a daily dose of 40 mg and TMZ in a daily dose of 10 mg, or IMP4297 in a daily dose of 20 mg and TMZ in a daily dose of 10 mg, or to provide IMPP4297 in a daily dose of 20-120 mg, such as 20-100 mg or 40-80 mg, and TMZ in a daily dose of 10-30 mg, such as 10-20 mg or 20-30 mg. In some embodiments, the amounts of the one or more pharmaceutical preparations of IMP4297 and one or more pharmaceutical preparations of TMZ in the kit are sufficient to provide the IMP4297 and TMZ for administration with any one of the above-mentioned daily doses for at least consecutive 7 days, such as at least consecutive 14 days or at least consecutive 21 days, or 28 days. In some embodiments, the amounts of the one or more pharmaceutical preparations of IMP4297 and one or more pharmaceutical preparations of TMZ in the kit are sufficient to provide the IMP4297 and TMZ for administration with any one of the above-mentioned daily doses for at least one cycle, such as 2-8 cycles, wherein one cycle includes consecutive 28 days with IMP4297 being administered for all 28 days and TMZ being administered for consecutive 21 days.

The pharmaceutical preparation of the present disclosure can also be a compound preparation containing both the effective dose of PARP inhibitors with trapping function and the non-toxic low dose of DNA damaging anti-cancer drugs as described in the disclosure.

The pharmaceutical preparations of the disclosure can be oral preparations, such as tablets, dragees, and capsules, as well as solutions suitable for injection or oral administration, containing from approximately 0.01% to 99%, in some embodiments, from approximately 0.25% to 75% of active compound(s), together with excipient(s).

The pharmaceutical preparations of the disclosure may be administered by any suitable means that achieve their intended purpose. For example, the pharmaceutical preparations may be administered by parenteral, subcutaneous, intravenous, intramuscular, intraperitoneal, transdermal, buccal, intrathecal, intracranial, intranasal or topical routes. Alternatively or additionally, the pharmaceutical preparations may be administered orally. The dosage administered will depend upon the age, health, and weight of the patient, the combined therapy, frequency of treatment, and the desired therapeutic efficacy, etc.

The pharmaceutical preparations of the present disclosure can be manufactured in a known manner, e.g., by conventional mixing, granulating, dragee-making, dissolving, or lyophilizing. Pharmaceutical preparations for oral use may be obtained by combining the active compounds with solid excipient(s), optionally grinding the resultant mixture, adding suitable auxiliaries if desired or necessary, processing the mixture of granules, thereby obtaining tablets or dragee cores.

Suitable excipients are, in particular, fillers, such as saccharides, e g, lactose or sucrose, mannitol or sorbitol; cellulose preparations and/or calcium phosphates, e.g. tricalcium phosphate or calcium hydrogen phosphate; as well as binders, such as starch paste, including maize starch, wheat starch, rice starch, potato starch, gelatin, tragacanth, methylcellulose, hydroxypropylmethylcellulose, sodium carboxymethylcellulose, and/or polyvinyl pyrrolidone. If desired, disintegrating agents may be added, which include but are not limited to the above-mentioned starches and carboxymethyl-starch, cross-linked polyvinyl pyrrolidone, agar, or alginic acid or a salt thereof, such as sodium alginate Auxiliaries are, in particular, flow-regulating agents and lubricants, e.g., silica, talc, stearic acid or salts thereof, such as magnesium stearate or calcium stearate, and/or polyethylene glycol. If desired, dragee cores can be provided with suitable coatings resistant to gastric juices. For this purpose, concentrated saccharide solutions may be used, which may optionally contain gum arabic, talc, polyvinyl pyrrolidone, polyethylene glycol and/or titanium dioxide, lacquer solutions and suitable organic solvents or solvent mixtures. In order to produce coatings resistant to gastric juices, solutions of suitable cellulose, such as acetylcellulose phthalate or hydroxypropylmethylcellulose phthalate, are used. Dye stuffs or pigments may be added to the tablets or dragee coatings, e.g., for identification or in order to characterize combinations of active compound doses.

Other pharmaceutical preparations, which may be administered orally, include push-fit capsules made of gelatin, as well as soft sealed capsules made of gelatin and a plasticizer, such as glycerol or sorbitol. The push-fit capsules may contain the active compounds in the form of granules, which may be mixed with fillers, such as lactose; binders, such as starches; and/or lubricants, such as talc or magnesium stearate; and stabilizers. In soft capsules, the active compound(s) are, in some embodiments, dissolved or suspended in suitable liquids, such as fatty oils or liquid paraffin, in which stabilizers may be added.

Suitable formulations for parenteral administration include aqueous solutions of the active compounds, e.g., aqueous solutions and alkaline solutions of water-soluble salts. In addition, suspensions of the active compounds as appropriate oily injection suspensions may be administered. Suitable lipophilic solvents or vehicles include fatty oils, e.g., sesame oil, or synthetic fatty acid esters, e.g, ethyl oleate, or triglycerides, or polyethylene glycol-400, or cremophor, or cyclodextrins. Aqueous injection suspensions may contain substances which increase the viscosity of the suspension, e.g, sodium carboxymethyl cellulose, sorbitol, and/or dextran. Optionally, suspension stabilizers may also be contained.

In some embodiments, the pharmaceutical preparation of the PARP inhibitor as described in the disclosure may be in a form of a solid dispersion. In some embodiments, the solid dispersion of a PARP inhibitor of the present disclosure contains an amorphous 5-fluoro-1-(4-fluoro-3-(4-(pyrimidin-2-yl)piperazine-1-carbonyl)benzyl)quinazoline-2,4(H,31-1)-dione and a polymer, wherein the polymer is hydroxypropyl methylcellulose acetate succinate or hydroxypropyl methylcellulose phthalate, and the polymer is present in an amount of 50% to 80% by weight, and wherein less than 10% by weight of 5-fluoro-1-(4-fluoro-3-(4-(pyrimidin-2-yl)piperazine-1-carbonyl)benzyl)quinazoline-2,4(1H,3H)-dione is crystalline. In some embodiments, the solid dispersion of a PARP inhibitor of the present disclosure contains an amorphous 5-fluoro-1-(4-fluoro-3-(4-(pyrimidin-2-yl)piperazine-1-carbonyl)benzyl)quinazoline-2,4(1H,3H)-dione and hydroxypropyl methylcellulose phthalate (in some embodiments, HP-55), wherein the hydroxypropyl methylcellulose phthalate is present in an amount of 50% to 80% by weight, in some embodiments, in an amount of 71% to 79% by weight, and wherein less than 10% by weight of 5-fluoro-1-(4-fluoro-3-(4-(pyrimidin-2-yl)piperazine-1-carbonyl) benzyl)quinazoline-2,4(1H,3H)-dione is crystalline.

In further embodiments, the pharmaceutical preparation of PARP inhibitors of the present disclosure is selected from:

(1) a pharmaceutical composition, comprising an amorphous solid dispersion powder of 5-fluoro-1-(4-fluoro-3-(4-(pyrimidin-2-yl)piperazine-1-carbonyl)benzyl)quinazoline-2,4(1H,3H)-dione, which accounts for 20 to 40% wt/wt of the pharmaceutical composition, and hydroxypropyl methylcellulose phthalate (in some embodiments, HP-55), which accounts for 60 to 80% wt/wt of the pharmaceutical composition, wherein the pharmaceutical composition is a solid dispersion obtained by spray drying;

(2) a pharmaceutical composition, comprising an amorphous solid dispersion powder of 5-fluoro-1-(4-fluoro-3-(4-(pyrimidin-2-yl)piperazine-1-carbonyl)benzyl)quinazoline-2,4(1H,3H)-dione, which accounts for about 25% wt/wt of the pharmaceutical composition, and hydroxypropyl methylcellulose phthalate HP-55, which accounts for about 75% wt/wt of the pharmaceutical composition;

(3) a pharmaceutical composition, comprising an amorphous solid dispersion powder of 5-fluoro-1-(4-fluoro-3-(4-(pyrimidin-2-yl)piperazine-1-carbonyl)benzyl)quinazoline-2,4(1H,3H)-dione, which accounts for about 33% wt/wt of the pharmaceutical composition, and hydroxypropyl methylcellulose phthalate HP-55, which accounts for about 67% wt/wt of the pharmaceutical composition;

(4) a pharmaceutical composition, comprising 5-fluoro-1-(4-fluoro-3-(4-(pyrimidin-2-yl)piperazine-1-carbonyl)benzyl)quinazoline-2,4(1H,3H)-dione, which accounts for about 25% wt/wt of the pharmaceutical composition, hydroxypropyl methylcellulose phthalate HP-55, which accounts for about 70% wt/wt of the pharmaceutical composition, and poloxamer, which accounts for about 5% wt/wt of the pharmaceutical composition.

The pharmaceutical preparations of PARP inhibitors suitable for the disclosure, especially solid dispersions, can be seen in PCT/CN2016/078262 (and US 2018/0071290 A1) for more details, and all the contents are included herein by reference.

In some embodiments of the disclosure, the PARP inhibitor is IMP4297 and the DNA damaging anti-cancer drug is TMZ. In some embodiments, the daily dose of IMP4297 is 20-120 mg and that of TMZ is 10-30 mg. In some embodiments, IMP4297 and TMZ are administered orally sequentially or simultaneously. The two can be prepared as separate preparations, or can be prepared as a compound preparation containing both. The preparation may contain one or more doses of drugs, as long as the contents of IMP4297 and TMZ in the one or more doses of drugs meet the daily dosage requirements described herein respectively.

In some embodiments, IMP4297 is administered in an amount of 40-80 mg daily for continuous 28 days from D1 to D28 (one cycle), and the DNA damaging anti-cancer drug TMZ is administered in an amount of 10-30 mg daily for continuous 21 days from D1 to D21 with no administration at D22 to D28 In some embodiments, an administration cycle is 28 days. The pharmaceutical preparations of the disclosure may be administered to any mammal, so long as they may experience the therapeutic effects of the compound(s) of the disclosure. Foremost among such mammals are humans and veterinary animals, although the disclosure is not intended to be so limited.

Use

In some embodiments, provided is one or more of the PARP inhibitors described herein, especially IMP4297, and one or more of DNA damaging anti-cancer drugs described herein, especially TMZ, for use in a method for treating one or more cancers described herein, wherein the method is as described in any one of the embodiments disclosed herein. In some embodiments, the method comprises orally administering a subject in need thereof. IMP4297 in a daily dose of 20-120 mg, such as 20-100 mg, such as 40 mg, 60 mg, 80 mg or 100 mg, for consecutive 28 days, and TMZ in a daily dose of 10-30 mg, such as 10 mg, 20 mg or 30 mg, for 21 days, with administration of 28 days as one cycle. In some embodiments, TMZ is administered daily and consecutively at day 1 to day 21 of the 28-day cycle.

Also provided is use of one or more of the PARP inhibitors described herein, especially IMP4297, and one or more of the DNA damaging anti-cancer drugs described herein, especially TMZ in the manufacture of a medicament or a kit for treating or preventing cancers described herein. Preferably, the kit is as described in any of the kit embodiments disclosed herein.

Preferably, the medicament or the kit contains one or more pharmaceutical compositions of the PARP inhibitor, especially IMP4297, and one or more pharmaceutical composition of the DNA damaging anti-cancer drug, especially TMZ, for administration of the PARP inhibitor, especially IMP4297, in a daily dose of 20-120 mg, such as 20-100 mg or 40-80 mg and the DNA damaging anti-cancer drug, especially TMZ, in a daily dose that is about 1/12th to about ⅕th of the daily dose of the DNA damaging anti-cancer drug when used alone or in combination with other drugs or therapies, such as 10-30 mg of TMZ.

Preferably, the medicament or the kit contains one or more pharmaceutical compositions of the PARP inhibitor, especially IMP4297, and one or more pharmaceutical composition of the DNA damaging anti-cancer drug, especially TMZ, for administration of the PARP inhibitor, especially IMP4297, in a daily dose of 20-120 mg, such as 20-100 mg or 40-80 mg and the DNA damaging anti-cancer drug, especially TMZ, in a daily dose of 10-30 mg, such as 10 mg, 20 mg or 30 mg, for at least consecutive 7 days, preferably for at least one cycle, wherein one cycle includes consecutive 28 days of daily administration of the PARP inhibitor, especially IMP4297 and consecutive 21 days of daily administration of the DNA damaging anti-cancer drug, especially TMZ

The present disclosure will be illustrated by way of specific examples below. It should be understood that these examples are merely illustrative and is not intended to limit the scope of the present disclosure. Unless otherwise specified, the materials and methods used in the examples are conventional materials and methods in the art.

### EXAMPLES

### Example 1: Inhibitory Effect of IMP4297 Combined with TMZ on the Growth of Human Small Cell Lung Cancer NCI-H209 Cells

The CCK-8 detection method was used to determine the inhibitory effect of IMP4297 combined with TMZ on the growth of human small cell lung cancer NCI-H209 cells. The resuscitated human small cell lung cancer NCI-H209 cells were inoculated into a culture dish, experimental medium (RPMI1640+20% FBS) was added, and the culture dish was incubated at 37° C. and 5% CO₂ in an incubator. Cells with good growth and suitable confluence were selected for the experiment, and centrifiged at 800 rpm for 5 min. The supernatant was discarded. The cells were resuspended with fresh medium and inoculated into a 96-well cell culture plate at a suitable cell density. 190 μL of cell suspension liquid was inoculated into each well. The test compound (including IMP4297, TMZ and reference compound AZD2281) was serially diluted with DMSO at a ratio of 1:3 and 1:10 to 10 concentrations (the last concentration is the DMSO negative control). 5 μL of each concentration was added to 120 μL of media (25 times dilution). The mixture was mixed well by shaking. Separate medication: 5 μL of the diluted medium containing the corresponding concentration of the compound and 5 μL of the medium were added respectively, combined medication. 5 μL of the diluted medium containing the corresponding concentration of the compound and 5 μL of the medium containing the final concentration of 50 μM of TMZ were added respectively. The final concentration of DMSO is 2%. The culture plate was then incubated at 37° C. and 5% CO₂ in an incubator for 5 days. 20 μL of CCK-8 detection reagent was added to each well, and the culture was continued for 2 hours. The culture plate was then shaked for 10 minutes and placed on a multi-function reader to measure the absorbance value (OD value) using the wavelength of 450/65011m. Graph Pad Prism 6.0 was used to analyze the data. The inhibitory effects of compounds on cell proliferation were plotted based on cell viability and the logarithm of compound concentration. Cell viability % (nM) OD_{compound}/OD_{DMSO}×100. The IC₅₀ values were fitted by a sigmoidal dose response curve equation Y=1001(1+10\hat{ }(Log C−Log IC₅₀)), wherein C was the concentration of the compound. The combination index was calculated using CalcuSyn software.

Table 1 summarizes the inhibitory effect (IC₅₀) of compounds on the proliferation of human small cell lung cancer NCI-H209 cells. Table 2 lists the combination index (CI) of IMP4297 and TMZ. CI<0.1 indicates that the drug combination has a strong synergistic effect. 0.1<CI<1 indicates that the drug combination has a synergistic effect, and CI>1 indicates that there is no synergistic effect.

TABLE 1
Inhibitory effects (IC₅₀) of compounds on the proliferation
of human small cell lung cancer NCI-H209 cells

| Compound | IC₅₀ (nM) |
|---|---|
| TMZ | >100000 |
| IMP4297 | 113.2 |
| 50 μM TMZ + IMP4297 | 6.847 |
| AZD2281 | 2176 |
| 50 μM TMZ + AZD2281 | 199.1 |

TABLE 2
The combination index (CI) of IMP4297 and TMZ

| C_{IMP4297} (nM) | C_{TMZ} (nM) | Cell viability % | CI |
|---|---|---|---|
| 0.1 | 50000 | 0.841 | 5.045 |
| 0.33 | 50000 | 0.8599 | 6.996 |
| 1 | 50000 | 0.8661 | 7.916 |
| 10 | 50000 | 0.2685 | 0.062 |
| 33 | 50000 | 0.139 | 0.065 |
| 100 | 50000 | 0.1382 | 0.191 |
| 333 | 50000 | 0.1238 | 0.548 |

Therefore, the results show that the combination of IMP4297 and TMZ has a strong synergistic effect on inhibiting the proliferation of human small cell lung cancer NCI-H209 cells.

### Example 2: The Effect of IMP4297 on PARP1 Trapping in DU145 Cells Treated with MMS

Human prostate cancer DU145 cells were used in the experiment. On the first day, cells were seeded to 10 cm cell culture dishes and kept in an incubator overnight. On the next day, vehicle (0.5% DMSO), and IMP4297 or Olaparib at 10 μM, 1 μM, 0.1 μM, 0.01 μM and 0 μM in the presence 0.01% MMS, was added to each culture dish respectively. Of note, 10% MMS was prepared fresh from 99% MMS in phosphate-buffered saline (PBS), and then diluted in culture medium to final concentration (0.01%). The plates were swirled, and the cells were kept in an incubator at 37° C., 5% CO₂ for 4 hours. Totally 12 samples were prepared and tested. After incubation, the nuclear chromosomal component was extracted according to subcellular protein fractionation kit instructions. Protein concentration was measured by Pierce® BCA Protein Assay Kit (Thermo) and protein was ready for western blot assay. 10 μg protein was loaded into each well of bolt gels, then wet transfer was performed, and the membrane was incubated overnight at 4° C. with anti-PARP antibody (Santa Cruz Biotechnology) with 1.500 dilution and 1:25000 dilution of anti-H3 antibody, respectively. The membrane was washed the next day with TBST 3 times, 5 minutes/time, then incubated with Goat anti-rabbit IgG-HRP (Santa Cruz Biotechnology) in a 1:4000 dilution for 1 hour at room temperature. ECL Prime Western Blotting Det kit (GE) was used to develop the target on the membrane. The grey density values of PARP1 bands were analyzed by ImageJ and summarized in Table 3.

TABLE 3
PARP1 trapping of IMP4297 and Olaparib in DU145 cells

| | 0.5% | MMS, 0.01% | | | | |
|---|---|---|---|---|---|---|
| Drugs | DMSO | 0 μmol/L | 0.01 μmol/L | 0.1 μmol/L | 1 μmol/L | 10 μmol/L |
| Olaparib | 90.302 | 238.73 | 590.88 | 750.61 | 1745.5 | 2960.9 |
| IMP4297 | 86.286 | 192.5 | 3667.63 | 4932.39 | 6036.79 | 5302.8 |

In this study, the trapping effect of IMP4297 to PARP1 on nuclear chromosome in DU145 cells treated with MMS was tested, and PARP inhibitor Olaparib was used as a positive control. The results showed that the trapping effect of Olaparib to PARP1 in nuclear chromosome is highly consistent with literature report. IMP4297 showed good PARP1 trapping in nuclear chromosome from 0.01-10 μM. In addition, the data showed that IMP4297 has unexpectedly stronger PARP1 trapping in nuclear chromosome than Olaparib under the same concentrations and conditions.

### Example 3: The Effect of IMP4297 on PARP1 Trapping in HCT116 Cells Treated with TMZ

Colorectal carcinoma HCT116 cells were used in the experiment Cells in exponential growth phase were trypsinized and seeded to 10 cm cell culture plate at a density about 10-20% confluency After two days when cell density reached 70-80%, the cell culture was replaced with fresh medium containing vehicle, 0.1 μM or 10 μM of IMP4297 either in the presence or absence of 1 mM TMZ. Cells were treated for 4 hours. Chromatin-bound proteins were extracted according to the “Subcellular Protein Fractionation Kit for Cultured Cells (Thermo, LOT78840)” protocol.

PARP trapping was detected using SDS-PAGE electrophoresis followed by Western blot Extracted chromatin-bound protein samples were mixed with 5×SDS-PAGE solution and heated for 5 min at 100° C. Equal amount (25-30 μg) of protein was loaded to each lane on a pre-made SDS-PAGE gel (Genscript SurePAGE™, Bis-Tris, 4-20%, 15 wells M00657). Anti-PARP1 antibody (ABCAM ab227244) used in the study was 1:1000 diluted, anti-Histone 113 antibody (ABCAM ab1791) was 1.5000 diluted and anti-rabbit IgG HRP-linked Antibody (CST 7074s) was 1:2000 diluted. The bands were detected by standard Western blot protocol.

The grey density values of PARP1 bands were analyzed by ImageJ and summarized in Table 4.

TABLE 4
The Effect of IMP4297 on PARP1 trapping
in HCT116 cells treated with TMZ

| | IMP4297 | | |
|---|---|---|---|
| Drug/Reagent | 0 μmol/L | 0.1 μmol/L | 10 μmol/L |
| Vehicle | 100 | 141 | 182 |
| TMZ, 1 mM | 193 | 398 | 446 |

In this study, the trapping effect of IMP4297 to PARP1 on nuclear chromosome in HCT116 cells treated with TMZ was tested. IMP4297 showed good PARP1 trapping in nuclear chromosome from 0.1-10 μM in HCT116 cells in the presence of TMZ.

### Example 4—in Nivo Efficacy Study of IMP4297 Combined with TMZ in NCI-H209 Human Small Cell Lung Cancer Xenograft Tumor Model in Nude Mice

The anti-tumor efficacy of the compound IMP4297 combined with TMZ was evaluated in the NCI-H209 human small cell lung cancer xenograft tumor model in nude mice. For this purpose, human small cell lung cancer NCI-H209 cells were inoculated into the subcutaneous breast area of the right axilla of nude mice. The cell inoculation amount was 2×10⁶ logarithmic growth phase cells. The inoculated mice were used after transplanted tumors were formed. Vigorously growing tumor tissues were cut into small pieces of 1×1×1 mm³, and inoculated under the skin of the breast area of the right axilla of each BALB/c nude mouse. When the average tumor volume reached about 124.08 (57.16-280.79) mm³, nude mice were randomly grouped according to the tumor volume and were administered the drugs. The grouping and dosing schedule are shown in Table 5.

TABLE 5
Animal grouping and dosing schedule for in vivo
efficacy study of IMP4297 in combination with TMZ

| | | | | | Dosing | | |
|---|---|---|---|---|---|---|---|
| | | Compound | Dosage | | volume | Route of | Dosing |
| Group | N | therapy | (mg/kg) | | (μL/g) | administration | regimes |
| 1 | 10 | Vehicle | — | | 20 | p.o | QD x21 D |
| 2 | 10 | TMZ | 3 | mg/kg | 20 | p.o | QD x 21 D |
| 3 | 10 | IMP4297 | 10 | mg/kg | 20 | p.o | QD x 21 D |
| 4 | 10 | IMP4297 + TMZ | 5 + 3 | mg/kg | 20 | p.o | QD x 21 D |
| 5 | 10 | IMP4297 + TMZ | 10 + 3 | mg/kg | 20 | p.o | QD x 21 D |

Note 1:
Administration was 20 μL/g based on the nude mice body weight.
Note 2:
N is the number of animals, 10 tumor-bearing nude mice per group, p.o. is oral administration; QD is once a day; continuous administration for 21 days.
Note 3:
The vehicle contained 10% DMSO in 10% HP-β-CD PBS.
Note 4:
The vehicle control group was given 20 μL/g of vehicle by gavage according to body weight, the TMZ and IMP4297 single agent -treated group was given 10 μL/g of vehicle by gavage first, and then given 10 μL/g of drug by gavage; the combination-treated group was given 10 μL/g of TMZ by gavage first, and then given 10 μL/g of IMP4297 by gavage after 30 minutes.

After grouping and drug treatment start, the weight of nude mice was weighed twice a week and recorded. Weight change (%)=(Wₜ−W₁)/W₁×100%, wherein W₁ is the body weight measured at the time of grouping administration (i.e D1), and Wₜ is the weight of the recording day. Weight change (%) is a measure of treatment-related toxicity (when average weight loss exceeded 15%, treatment was stopped or the schedule was adjusted until recovery; when average weight loss exceeded 20%, the experiment was terminated). The tumor diameter (length and width) was measured twice a week with a vernier caliper. The tumor volume (length×width²/2) and the relative tumor volume RTV=Vₜ/V₁ were calculated, wherein V₁ is the tumor volume at the time of grouping administration (i.e. D1), and Vₜ is the tumor volume at each measurement. The evaluation index of anti-tumor efficacy is expressed as relative tumor growth rate T/C (%) and tumor growth inhibition rate TGI (%) T/C(%)=T_{RTV}/C_{RTV}×100°, wherein T_{RTV} is RTV of the treatment group, compounds with a T/C(%) lower than 50 are defined as active (effective), and C_{RTV} is RTV of the vehicle control group. TGM(%)=[(CVₜ−CV₁)−(TVₜ−TV₁)]/(CVₜ−CV₁)×100%, wherein CV₁ is the tumor volume on the recording day of the vehicle control group, CV₁ is the tumor volume of the vehicle control group at the time of grouping administration, TVₜ is the tumor volume of the drug administration group on the recording day, and TV₁ is the tumor volume of the drug administration group at the time of grouping administration.

After the experiment, 3 nude mice were randomly selected from each group, and 300 μL of whole blood was collected from the orbit and placed in a BD K₂EDTA anticoagulant tube (REF367841) for routine blood testing.

GraphPad Prism 6.0 software two-way ANOVA was used to compare the mean tumor volume and relative tumor volume between groups. Compared with the control group (vehicle), *p<0.05 (statistical difference), **p<0.01 (significant statistical difference), ***p<0.001 (very significant statistical difference); compared with the TMZ group, ^{#}p<0.05 (statistical difference), ^{##}p<0.01 (significant statistical difference), ^{###}p<0.001 (very significant statistical difference); compared with the IMP4297 group, ⁺p<0.05 (statistical difference), ⁺⁺p<0.01 (significant statistical difference), ⁺⁺⁺p<0.001 (very significant statistical difference).

Experimental Results

1. Weight

During the experiment, there was no body weight reduction in the TMZ single agent-treated group, as well as in the FMP4297 single agent-treated group. The body weight of the two combination treatment groups (IMP4297 5 mg/kg+TMZ 3 mg/kg and IMP4297 10 mg/kg+TMZ 3 mg/kg) decreased slightly (less than 5%)

2. Tumor Volume

Compared with the vehicle-treated control group, the TMZ 3 mg/kg and IMP4297 10 mg/kg single agent-treated groups showed no significant inhibitory effect on tumor growth. The two combination-treated groups had a very significant inhibitory effect on tumor growth (p<0.0001). Compared with the single agent-treated group, the combination of TMZ with IMP4297 showed a significant synergistic effect in the two combination-treated groups (p<0.0001). The tumor volume in the combination-treated group of IMP4297 10 mg/kg and TMZ 3 mg/kg decreased compared with day 0.

3. Evaluation Index of Anti-Tumor Efficacy

The relative tumor growth rate [TIC(%)] and tumor growth inhibition rate [TGI(%)] of each group during the experiment are shown in Table 6 below.

TABLE 6
Evaluation index of anti-tumor efficacy

| | | | | Average |
|---|---|---|---|---|
| | | | | weight |
| Group | Compound therapy | TGI (%) | T/C (%) | change (%) |
| 1 | Vehicle | — | — | 9.80 ± 5.68 |
| 2 | TMZ (mpk, QD) | 31.66**** | 64.03**** | 7.99 ± 5.21 |
| 3 | IMP4297 (10 mpk, QD) | 18.80** | 79.12** | 5.76 ± 3 92 |
| 4 | IMP4297 + TMZ (5 + 3 mpk, QD) | 98.03****^{####++++} | 16.43****^{####++++} | 0.40 ± 4.79 |
| 5 | IMP4297 + TMZ (10 + 3 mpk, QD) | 109.93****^{####++++} | 3.25****^{####++++} | −3.60 ± 3.72 |

Note 1:
Compared with the control group (vehicle), *p < 0.05, **p < 0.01, ***p < 0.001, ****p < 0.0001; compared with the TMZ group, ^{#}p < 0.05, ^{##}p < 0.01, ^{###}p < 0.001, ^{####}p < 0.0001; compared with the IMP4297 group, ⁺p < 0.05, ⁺⁺p < 0.01, ⁺⁺⁺p < 0.001, ⁺⁺⁺⁺p < 0.0001.
Note 2:
Average weight change (%): The average weight change (%) of nude mice in each group compared with the initial weight at the beginning of the treatment.

4. Blood Test Results

There were no significant reductions in red blood cells in all drug-treated groups, there were no significant change in platelets, with a slight increase in the combination treated group, white blood cells were decreased in all drug-treated groups. Although the reduction of white blood cells was more obvious in the combination treated groups, the decrease was not significant.

In summary, EMP4297 combined with TMZ were administered once daily continuously for 21 consecutive days, showed a very significant anti-tumor effect on NCI-1209 human small cell lung cancer xenograft model. The combination of two anti-tumor drugs showed a significant synergistic effect, while the high-dose combination group only had a slight decrease in body weight, and no significant hemocological changes were observed.

### Example 5: a Phase Ib/II Clinical Trial of IMP4297 in Combination with Temozolomide in Patients with Advanced Solid Tumors

Methods:

Patients with advanced solid tumors were treated with IMP4297 (QD continuously for days 1-28) in combination with TMZ (QD continuously for days 1-21 with days 22-28 off of a 28-day cycle) (ClinicalTrials.gov Identifier: NCT04434482)

Results:

7 Patients have been enrolled in 3 cohorts (Cohort 1: IMP4297 40 mg+TMZ 20 mg, one patient; Cohort 2: IMP4297 60 mg+TMZ 20 mg, three patients; Cohort 3: IMP4297 80 mg+TMZ 20 mg, three patients), including patients with pancreatic (1), endometrial (1), ovarian (1), mesothelioma (1), small-cell lung (1), rectal (1), and peripheral nerve sheath (1) cancers No dose-limiting toxicities (DLTs) were reported in these 7 patients. The clinical trial is ongoing, and the maximum tolerated dose (MTD) and the recommended Phase II dose (RP2D) of IMP4297 in combination with temozolomide has not been determined.

Tumor assessments were available in all these 7 patients. There were 1 patient with partial response (PR) in Cohort 1; 3 patients with stable disease (SD) in Cohort 2 (1 patient was out of the study after one tumor assessment due to non-treatment related reasons); 1 patient with PR, 1 patient with SD and 1 with progressive disease (PD, new lesion and out of the study) in Cohort 3. The results are as follow:
1 PR was confirmed in the patient with pancreatic cancer enrolled in Cohort 1. The patient had PR after 14 line treatment but could not tolerate the treatment. The target lesions started to regress, and the non-target lesions disappeared after 2 cycles of treatment. PR was observed after 6 cycles of treatment, and confirmed PR (target disease reduction of 43.3%) was observed after 8 cycles of treatment. The patient is in treatment with continuous response at cycle 12 now.
1 SD was observed in the patient with advanced endometrial carcinoma enrolled in Cohort 2 The patient is heavily pretreated and had received at least 4 prior lines of chemotherapies and/or immunotherapy. The target lesions started to regress after 2 cycles of treatment and had 28.8% reduction after 8 cycles of treatment. The patient is in treatment at cycle 10 now.
1 SD was observed in the patient with advanced ovarian cancer enrolled in Cohort 2. The patient had received at least 3 prior lines of chemotherapies. The target lesions showed 20% reduction after 4 cycles of treatment. The patient is in treatment at cycle 9 now.
1 PR was observed in the patient with small cell lung cancer enrolled in Cohort 3 The target lesions started to regress after 2 cycles of treatment (27% reduction), and PR (target disease reduction of 38.5%) after 4 cycles of treatment. The patient is in treatment at cycle 5 now.
1 SD was observed in the patient with rectal cancer enrolled in Cohort 3. The patient is in treatment at cycle 5 with SD now.

23 adverse events (AEs) were reported from the 7 patients, 16 of which were treatment emergent adverse events (TEAEs) And 5 of 16 TEAEs were judged as related to investigational drug(s). The most common TEAEs reported were hematological toxicity, including 1 anemia (Grade 3), 1 pancytopenia (Grade 2).

### CONCLUSION

The results presented here indicate that the combination of high dose IMP4297 with low dose temozolomide using the continuous dosing schedule had good tolerability (long duration of treatment as well as duration of response, one patient have been treated for over 11 cycles, one over 9 cycles and one over 8 cycles, 2 over 4 cycles among 7 patients) and good efficacy (2 PR as well as 3 SD with tumor reduction among 7 patients) in patients with different types of advanced solid tumors. Results demonstrate that this is a promising therapeutic combination, and further investigation of this combination in patients with a variety of cancers is ongoing.

1. A method of treating cancer in a subject, the method comprising administering one or more PARP inhibitors and one or more DNA damaging anti-cancer drugs, or a compound preparation comprising the one or more PARP inhibitors and one or more DNA damaging anti-cancer drugs, wherein the PARP inhibitors are selected from a group consisting of compounds represented by Formula I: 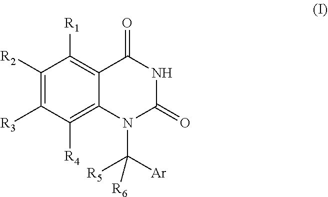
or pharmaceutically acceptable salts, solvates or prodrugs thereof, wherein:
Ar is an optionally substituted aryl or an optionally substituted heteroaryl; and
R₁-R₆ are independently hydrogen, halo, optionally substituted amino, optionally substituted alkoxy, optionally substituted C₁₋₁₀ alkyl, alkenyl, alkynyl, nitro, cyano, acylamido, hydroxy, thiol, acyloxy, azido, carboxy, ethylenedioxo, hydroxylamido or optionally substituted alkylthiol.

2. The method of claim 1, wherein the PARP inhibitor is selected from a group consisting of compounds represented by Formula II: 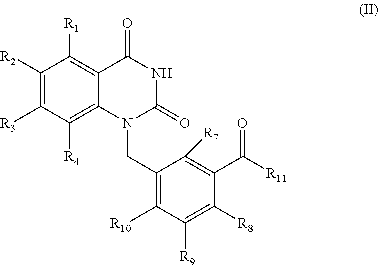
or pharmaceutically acceptable salts, solvates or prodrugs thereof, wherein:
R₁-R₄ are independently hydrogen, halo, optionally substituted amino, optionally substituted alkoxy, optionally substituted C₁₋₁₀ alkyl, alkenyl, alkynyl, nitro, cyano, acylamido, hydroxy, thiol, acyloxy, azido, carboxy, ethylenedioxo, hydroxylamido or optionally substituted alkylthiol;
R₇-R₁₀ are independently hydrogen, halo, optionally substituted amino, alkoxy, C₁₋₁₀ alkyl, haloalkyl, aryl, heteroaryl, carbocyclic group, heterocyclic group, alkenyl, alkynyl, arylalkyl, arylalkenyl, arylalkynyl, heteroarylalkyl, heteroarylalkenyl, heteroarylalkynyl, carbocycloalkyl, heterocycloalkyl, hydroxyalkyl, hydroxyalkoxy, aminoalkyl, aminoalkoxy, carboxyalkyl, carboxyalkoxy, nitro, cyano, acylamido, aminocarbonyl, hydroxy, thiol, acyloxy, azido, carboxy, hydroxylamido, alkylsulfonyl, aminosulfonyl, di-substituted alkylaminosulfonyl, alkylsulfiniyl, alkylthiol, or substituted carbonyl; and
R₁₁ is an optionally substituted amino, hydrazine, alkoxy, C₁₋₁₀ alkyl, haloalkyl, aryl, heteroaryl, carbocyclic group, heterocyclic group, alkenyl, alkynyl, arylalkyl, arylalkenyl, arylalkynyl, heteroarylalkyl, heteroarylalkenyl, heteroarylalkynyl, carbocycloalkyl, heterocycloalkyl, hydroxyalkyl, hydroxyalkoxy, aminoalkyl, aminoalkoxy, carboxyalkyl, carboxyalkoxy, acylamido, hydroxy, thiol, acyloxy, hydroxylamido, or alkylthiol.

3. The method of claim 1, wherein the PARP inhibitor is selected from a group consisting of compounds represented by Formula III: 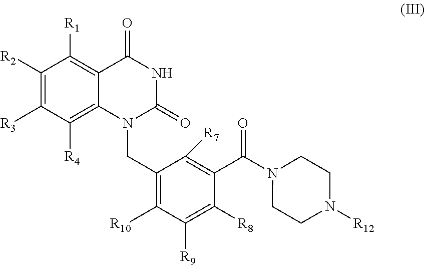
or pharmaceutically acceptable salts, solvates or prodrugs thereof, wherein:
R₁-R₄ are independently hydrogen, halo, optionally substituted amino, optionally substituted alkoxy, optionally substituted C₁₋₁₀ alkyl, alkenyl, alkynyl, nitro, cyano, acylamido, hydroxy, thiol, acyloxy, azido, carboxy, ethylenedioxo, hydroxylamido or optionally substituted alkylthiol;
R₇-R₁₀ are independently hydrogen, halo, optionally substituted amino, alkoxy, C₁₋₁₀ alkyl, haloalkyl, aryl, heteroaryl, a carbocyclic group, a heterocyclic group, alkenyl, alkynyl, arylalkyl, arylalkenyl, arylalkynyl, heteroarylalkyl, heteroarylalkenyl, heteroarylalkynyl, carbocycloalkyl, heterocycloalkyl, hydroxyalkyl, hydroxyalkoxy, aminoalkyl, aminoalkoxy, carboxyalkyl, carboxyalkoxy, nitro, cyano, acylamido, aminocarbonyl, hydroxy, thiol, acyloxy, azido, carboxy, hydroxylamido, alkylsulfonyl, aminosulfonyl, di-substituted alkylaminosulfonyl, alkylsulfiniyl, alkylthiol, or substituted carbonyl; and
R₁₂ is an optionally substituted C₁₋₁₀ alkyl, cycloalkyl, aryl, heteroaryl, carbocyclic group, heterocyclic group, alkenyl, alkynyl, acyl, arylalkenyl, arylalkynyl, heteroarylalkenyl, heteroarylalkynyl, alkylcarbonyl, cycloalkylcarbonyl, arylcarbonyl, heteroarylcarbonyl, heterocyclocarbonyl, aminocarbonyl, alkylsulfonyl, cycloalkylsulfonyl or aminosulfonyl.

4. The method Use of claim 3, wherein,
R₁ and R₂ are independently selected from the group consisting of H, halo, C₁₋₆ alkyl and C₁₋₆ alkoxy;
R₃ is H;
R₄ is selected from the group consisting of H, halo, C₁₋₆ alkyl, C₁₋₆ alkoxy and hydroxy;
R₇, R₈, R₉ and R₁₀ are independently hydrogen or halo; and
R₁₂ is an optionally substituted cycloalkyl, aryl, heteroaryl, carbocyclic group, heterocyclic group, arylalkyl, heteroarylalkyl, carbocycloalkyl, heterocycloalkyl, alkylcarbonyl, cycloalkylcarbonyl, arylcarbonyl, heteroarylcarbonyl or heterocyclocarbonyl; or
R₁ and R₂ are independently selected from the group consisting of hydrogen, fluoro, chloro, bromo and methyl;
R₄ is hydrogen, fluoro, methoxy or hydroxyl;
R₇, R₈, R₉ and R₁₀ are independently hydrogen or halo; and
R₁₂ is an optionally substituted C₃₋₈ cycloalkyl, pyridyl, pyrimidinyl, benzoyl, phenyl, piperidinyl, thienylcarbonyl, furylcarbonyl, piperazinyl or thiazolyl; or
R₁ is halo, NH₂, C₁₋₆ alkyl, nitro or hydroxy, and R₂-R₄ are independently hydrogen, halo, NH₂, C₁₋₆ alkyl, nitro or hydroxy; or R₂ is halo, NH₂, C₁₋₆ alkyl, nitro or hydroxy, and R₁ and R₃-R₄ are independently hydrogen, halo, NH₂, C₁₋₆ alkyl, nitro or hydroxy; or R₃ is halo, NH₂, C₁₋₆ alkyl, nitro or hydroxyl, and R₁, R₂ and R₄ are independently hydrogen, halo, NH₂, C₁₋₆ alkyl, nitro or hydroxy;
or R₄ is halo, NH₂, C₁₋₆ alkyl, nitro or hydroxyl, and R₁-R₃ are independently hydrogen, halo, NH₂, C₁₋₆ alkyl, nitro or hydroxyl;
R₇-R₁₀ are independently hydrogen, halo, C₁₋₆ alkoxy or nitro; and
R₁₂ is pyridinyl, pyrimidinyl, C₃₋₈ cycloalkyl, C₁₋₆ alkyl optionally substituted by one C₃₋₈ cycloalkyl, benzoyl optionally substituted by one or more substituents selected from halo and C₁₋₆ alkoxy, carbonyl optionally substituted by one substituent selected from C₃₋₈ cycloalkyl, thiophenyl, pyridinyl, furanyl and tetrahydrofuranyl, C₁₋₆ alkylsulfonyl, phenyl, pyrazinyl, benzo[d]isothiazol-3-yl, benzoisoxazolyl optionally substituted by one or more halogens, and thiazolyl; or
R₁ is halo or C₁₋₆ alkyl, R₂ is hydrogen, halo, —NH₂ or C₁₋₆ alkyl, R₃ is hydrogen, halo, C₁₋₆ alkoxy or C₁₋₆ alkyl, and R₄ is hydrogen, halo, hydroxy, C₁₋₆ alkoxy or C₁₋₆ alkyl; or R₁ is hydrogen, halo or C₁₋₆ alkyl, R₂ is halo, —NH₂ or C₁₋₆ alkyl, R₃ is hydrogen, halo, C₁₋₆ alkoxy or C₁₋₆ alkyl, and R₄ is hydrogen, halo, hydroxy, C₁₋₆ alkoxy or C₁₋₆ alkyl; or R₁ is hydrogen, halo or C₁₋₆ alkyl, R₂ is hydrogen, halo, —NH₂ or C₁₋₆ alkyl, R₃ is halo, C₁₋₆ alkoxy or C₁₋₆ alkyl, and R₄ is hydrogen, halo, hydroxy, C₁₋₆ alkoxy or C₁₋₆ alkyl; or R₁ is hydrogen, halo or C₁₋₆ alkyl, R₂ is hydrogen, halo, —NH₂ or C₁₋₆ alkyl, R₃ is hydrogen, halo, C₁₋₆ alkoxy or C₁₋₆ alkyl, and R₄ is halo, hydroxy, C₁₋₆ alkoxy or C₁₋₆ alkyl;
R₇-R₁₀ are independently hydrogen, halogen, or C₁₋₆ alkyl; and
R₁₂ is C₃₋₈ cycloalkyl, C₃₋₈ cycloalkylcarbonyl, pyridyl, pyrimidinyl, benzoyl, phenyl, piperidinyl, thiophenylcarbonyl, furanyl or pyrazinyl, which is optionally substituted by 1, 2, 3, or 4 substituents selected from the group consisting of halo and C₁₋₆ alkyl; or
R₁ is halo;
R₂ is hydrogen, halo or C₁₋₆ alkyl;
R₃ is hydrogen, halo or C₁₋₆ alkyl;
R₄ is hydrogen, halo or C₁₋₆ alkyl;
R₇-R₁₀ are independently hydrogen or halo; and
R₁₂ is pyrimidinyl; or
R₁ is fluoro, chloro, bromo or methyl;
R₂ is hydrogen, fluoro, chloro, bromo or methyl;
R₃ is hydrogen, fluoro, chloro or methyl;
R₄ is hydrogen, fluoro, chloro, methoxy or methyl; and
R₇, R₈, R₉ and R₁₀ are independently hydrogen or fluoro; or
R₂ is fluoro, chloro, bromo or methyl;
R₁ is hydrogen, fluoro, chloro, bromo or methyl;
R₃ is hydrogen, fluoro, chloro or methyl;
R₄ is hydrogen, fluoro, methoxy or hydroxyl; and
R₇, R₈, R₉ and R₁₀ are independently hydrogen or fluoro; or
R₄ is fluoro, methoxy or hydroxyl;
R₁ is hydrogen, fluoro, chloro, bromo or methyl;
R₂ is hydrogen, fluoro, chloro, bromo or methyl;
R₃ is hydrogen, fluoro, chloro or methyl; and
R₇, R₈, R₉ and R₁₀ are independently hydrogen or fluoro.

5. The method of claim 1, wherein the PARP inhibitor is selected from the group consisting of:
1-(3-(4-(Pyridin-2-yl)piperazine-1-carbonyl)benzyl)quinazoline-2,4(1H,3H)-dione;
1-(3-(4-(Pyrimidin-2-yl)piperazine-1-carbonyl)benzyl)quinazoline-2,4(1H,3H)-dione;
1-(3-(4-Cyclohexylpiperazine-1-carbonyl)benzyl)quinazoline-2,4(1H,3H)-dione;
1-(3-(4-([1,2,4]Triazolo[4,3-b]pyridazin-6-yl)piperazine-1-carbonyl)benzyl)quinazoline-2,4(1H,3H)-dione;
1-(3-(4-Ethylpiperazine-1-carbonyl)benzyl)quinazoline-2,4(1H,3H)-dione;
1-(3-(4-Benzoylpiperazine-1-carbonyl)benzyl)quinazoline-2,4(1H,3H)-dione;
1-(3-(4-(4-Fluorobenzoyl)piperazine-1-carbonyl)benzyl)quinazoline-2,4(1H,3H)-dione;
1-(3-(4-(4-Chlorobenzoyl)piperazine-1-carbonyl)benzyl)quinazoline-2,4(1H,3H)-dione;
1-(3-(4-(4-Bromobenzoyl)piperazine-1-carbonyl)benzyl)quinazoline-2,4(1H,3H)-dione;
1-(3-(4-(4-Methoxybenzoyl)piperazine-1-carbonyl)benzyl)quinazoline-2,4(1H,3H)-dione;
1-(3-(4-(Tetrahydro-2H-pyran-4-yl)carbonylpiperazine-1-carbonyl)benzyl)quinazoline-2,4(1H,3H)-dione;
1-(3-(4-(Cyclopentylcarbonyl)piperazine-1-carbonyl)benzyl)quinazoline-2,4(1H,3H)-dione;
1-(3-(4-(Cyclopropylcarbonyl)piperazine-1-carbonyl)benzyl)quinazoline-2,4(1H,3H)-dione;
1-(3-(4-(Ethylsulfonyl)piperazine-1-carbonyl)benzyl)quinazoline-2,4(1H,3H)-dione;
1-(3-(4-Acetylpiperazine-1-carbonyl)benzyl)quinazoline-2,4(1H,3H)-dione;
1-(3-(4-Phenylpiperidine-1-carbonyl)benzyl)quinazoline-2,4(1H,3H)-dione;
1-(3-(4-Phenylpiperazine-1-carbonyl)benzyl)quinazoline-2,4(1H,3H)-dione;
1-(3-(4-(Pyrazin-2-yl)piperazine-1-carbonyl)benzyl)quinazoline-2,4(1H,3H)-dione;
1-(4-Fluoro-3-methoxycarbonylbenzyl)quinazoline-2,4(1H,3H)-dione;
1-(3-Carbonyl-4-fluorobenzyl)quinazoline-2,4(1H,3H)-dione;
1-(4-Fluoro-3-(4-(Cyclopentylcarbonyl)piperazine-1-carbonyl)benzyl)quinazoline-2,4(1H,3H)-dione;
1-(4-Fluoro-3-(4-(pyridin-2-yl)piperazine-1-carbonyl)benzyl)quinazoline-2,4(1H,3H)-dione;
1-(4-Fluoro-3-(4-(pyrimidin-2-yl)piperazine-1-carbonyl)benzyl)quinazoline-2,4(1H,3H)-dione;
1-(3-(4-(Cyclohexylcarbonyl)piperazine-1-carbonyl)benzyl)quinazoline-2,4(1H,3H)-dione;
1-(3-(4-(Benzo[d]isothiazol-3-yl)piperazine-1-carbonyl)benzyl)quinazoline-2,4(1H,3H)-dione;
1-(3-(4-(Piperidin-1-yl)piperidine-1-carbonyl)benzyl)quinazoline-2,4(1H,3H)-dione;
1-(3-(4-(Pyridin-4-yl)piperazine-1-carbonyl)benzyl)quinazoline-2,4(1H,3H)-dione;
1-(3-(4-(Cyclobutylcarbonyl)piperazine-1-carbonyl)benzyl)quinazoline-2,4(1H,3H)-dione;
1-(3-(4-(6-Fluorobenzo[d]isoxazol-3-yl)piperidine-1-carbonyl)benzyl)quinazoline-2,4(1H,3H)-dione;
1-(3-(4-(Thiophene-2-carbonyl)piperazine-1-carbonyl)benzyl)quinazoline-2,4(1H,3H)-dione;
1-(3-(4-(Furan-2-carbonyl)piperazine-1-carbonyl)benzyl)quinazoline-2,4(1H,3H)-dione;
1-(3-(4-(Furan-3-carbonyl)piperazine-1-carbonyl)benzyl)quinazoline-2,4(1H,3H)-dione;
1-(3-(4-(Thiophene-3-carbonyl)piperazine-1-carbonyl)benzyl)quinazoline-2,4(1H,3H)-dione;
1-(3-(4-(Pyridine-3-carbonyl)piperazine-1-carbonyl)benzyl)quinazoline-2,4(1H,3H)-dione;
1-(3-(4-(Pyridine-2-carbonyl)piperazine-1-carbonyl)benzyl)quinazoline-2,4(1H,3H)-dione;
1-(3-(4-(Pyridine-4-carbonyl)piperazine-1-carbonyl)benzyl)quinazoline-2,4(1H,3H)-dione;
1-(3-(4-Phenoxypiperidine-1-carbonyl)benzyl)quinazoline-2,4(1H,3H)-dione;
1-(4-Fluoro-3-(4-(cyclopropylcarbonyl)piperazine-1-carbonyl)benzyl)quinazoline-2,4(1H,3H)-dione;
1-(4-Fluoro-3-(4-(cyclohexylcarbonyl)piperazine-1-carbonyl)benzyl)quinazoline-2,4(1H,3H)-dione;
1-(4-Fluoro-3-(4-(pyrazin-2-yl)piperazine-1-carbonyl)benzyl)quinazoline-2,4(1H,3H)-dione;
1-(4-Fluoro-3-(4-(benzo[d]isothiazol-3-yl)piperazine-1-carbonyl)benzyl)quinazoline-2,4(1H,3H)-dione;
1-(4-Fluoro-3-(4-(cyclobutylcarbonyl)piperazine-1-carbonyl)benzyl)quinazoline-2,4(1H, 3H)-dione;
1-(4-Fluoro-3-(4-benzoylpiperazine-1-carbonyl)benzyl)quinazoline-2,4(1H,3H)-dione;
1-(4-Fluoro-3-(4-(thiophene-2-carbonyl)piperazine-1-carbonyl)benzyl)quinazoline-2,4(1H,3H)-dione;
1-(4-Fluoro-3-(4-(furan-2-carbonyl)piperazine-1-carbonyl)benzyl)quinazoline-2,4(1H,3H)-dione;
1-(6-Fluoro-3-(4-(cyclopentylcarbonyl)piperazine-1-carbonyl)benzyl)quinazoline-2,4(1H,3H)-dione;
1-(6-Fluoro-3-(4-(cyclohexylcarbonyl)piperazine-1-carbonyl)benzyl)quinazoline-2,4(1H, 3H)-dione;
1-(6-Fluoro-3-(4-(pyrimidin-2-yl)piperazine-1-carbonyl)benzyl)quinazoline-2,4(1H,3H)-dione;
1-(6-Fluoro-3-(4-(pyridin-2-yl)piperazine-1-carbonyl)benzyl)quinazoline-2,4(1H,3H)-dione;
1-(6-Fluoro-3-(4-(thiophene-2-carbonyl)piperazine-1-carbonyl)benzyl)quinazoline-2,4(1H,3H)-dione;
1-(6-Fluoro-3-(4-(cyclobutylcarbonyl)piperazine-1-carbonyl)benzyl)quinazoline-2,4(1H, 3H)-dione;
1-(6-Fluoro-3-(4-(furan-2-carbonyl)piperazine-1-carbonyl)benzyl)quinazoline-2,4(1H,3H)-dione;
1-(6-Chloro-3-(4-(cyclopentylcarbonyl)piperazine-1-carbonyl)benzyl)quinazoline-2,4(1H,3H)-dione;
1-(6-Chloro-3-(4-(pyrimidin-2-yl)piperazine-1-carbonyl)benzyl)quinazoline-2,4(1H,3H)-dione;
1-(6-Chloro-3-(4-(cyclohexylcarbonyl)piperazine-1-carbonyl)benzyl)quinazoline-2,4(1H, 3H)-dione;
1-(6-Chloro-3-(4-(pyridin-2-yl)piperazine-1-carbonyl)benzyl)quinazoline-2,4(1H,3H)-dione;
1-(2-Chloro-3-(4-(cyclopentylcarbonyl)piperazine-1-carbonyl)benzyl)quinazoline-2,4(1H,3H)-dione;
1-(2-Chloro-3-(4-(pyrimidin-2-yl)piperazine-1-carbonyl)benzyl)quinazoline-2,4(1H,3H)-dione;
1-(2-Fluoro-3-(4-(pyrimidin-2-yl)piperazine-1-carbonyl)benzyl)quinazoline-2,4(1H,3H)-dione;
1-(2-Fluoro-3-(4-(cyclopentylcarbonyl)piperazine-1-carbonyl)benzyl)quinazoline-2,4(1H,3H)-dione;
1-(5-Fluoro-3-(4-(pyrimidin-2-yl)piperazine-1-carbonyl)benzyl)quinazoline-2,4(1H,3H)-dione;
1-(5-Fluoro-3-(4-(cyclopentylcarbonyl)piperazine-1-carbonyl)benzyl)quinazoline-2,4(1H,3H)-dione;
1-(4-Chloro-3-(4-(pyrimidin-2-yl)piperazine-1-carbonyl)benzyl)quinazoline-2,4(1H,3H)-dione;
1-(4-Chloro-3-(4-(cyclohexylcarbonyl)piperazine-1-carbonyl)benzyl)quinazoline-2,4(1H, 3H)-dione;
1-((2-(4-(Cyclopentylcarbonyl)piperazine-1-carbonyl)furan-5-yl)methyl)quinazoline-2, 4(1H,3H)-dione;
1-((2-(4-(Benzo[d]isothiazol-3-yl)piperazine-1-carbonyl)furan-5-yl)methyl)quinazoline-2,4(1H,3H)-dione;
1-((2-(4-(Cyclopentylcarbonyl)piperazine-1-carbonyl)pyridin-6-yl)methyl)quinazoline-2,4(1H,3H)-dione;
1-((2-(4-(Pyridin-2-yl)piperazine-1-carbonyl)pyridin-6-yl)methyl)quinazoline-2,4(1H,3H)-dione;
1-((2-(4-(Pyrimidin-2-yl)piperazine-1-carbonyl)pyridin-6-yl)methyl)quinazoline-2,4(1H, 3H)-dione;
1-(3-(4-(Thiazol-2-yl)piperazine-1-carbonyl)benzyl)quinazoline-2,4(1H,3H)-dione;
1-(3-(4-(Cyclohexylmethyl)piperazine-1-carbonyl)benzyl)quinazoline-2,4(1H,3H)-dione;
1-(3-(4-(Cyclopentylmethyl)piperazine-1-carbonyl)benzyl)quinazoline-2,4(1H,3H)-dione;
1-(3-(4-(Cyclohexylsulfonyl)piperazine-1-carbonyl)benzyl)quinazoline-2,4(1H,3H)-dione;
7-Fluoro-1-(3-(4-(cyclopentylcarbonyl)piperazine-1-carbonyl)benzyl)quinazoline-2,4(1H,3H)-dione;
7-Fluoro-1-(3-(4-(pyrimidin-2-yl)piperazine-1-carbonyl)benzyl)quinazoline-2,4(1H,3H)-dione;
7-Methyl-1-(3-(4-(cyclopentylcarbonyl)piperazine-1-carbonyl)benzyl)quinazoline-2,4(1H,3H)-dione;
7-Methyl-1-(3-(4-(pyrimidin-2-yl)piperazine-1-carbonyl)benzyl)quinazoline-2,4(1H,3H)-dione;
6-Methyl-1-(3-(4-(pyrimidin-2-yl)piperazine-1-carbonyl)benzyl)quinazoline-2,4(1H,3H)-dione;
6-Methyl-1-(3-(4-(cyclopentylcarbonyl)piperazine-1-carbonyl)benzyl)quinazoline-2,4(1H,3H)-dione;
6-Methyl-1-(4-Fluoro-3-(4-(pyrimidin-2-yl)piperazine-1-carbonyl)benzyl)quinazoline-2,4 (1H,3H)-dione;
6-Methyl-1-(4-fluoro-3-(4-(cyclohexylcarbonyl)piperazine-1-carbonyl)benzyl)quinazoline-2,4(1H,3H)-dione;
6-Methyl-1-(4-fluoro-3-(4-(cyclopentylcarbonyl)piperazine-1-carbonyl)benzyl)quinazoline-2,4(1H,3H)-dione;
6-Bromo-1-(3-(4-(cyclohexylcarbonyl)piperazine-1-carbonyl)benzyl)quinazoline-2,4(1H, 3H)-dione;
6-Bromo-1-(3-(4-(cyclopentylcarbonyl)piperazine-1-carbonyl)benzyl)quinazoline-2,4(1H,3H)-dione;
6-Bromo-1-(3-(4-(pyrimidin-2-yl)piperazine-1-carbonyl)benzyl)quinazoline-2,4(1H,3H)-dione;
6-Nitro-1-(3-(4-(cyclopentylcarbonyl)piperazine-1-carbonyl)benzyl)quinazoline-2,4(1H, 3H)-dione;
6-Nitro-1-(3-(4-(pyrimidin-2-yl)piperazine-1-carbonyl)benzyl)quinazoline-2,4(1H,3H)-dione;
6-Nitro-1-(3-(4-(cyclohexylcarbonyl)piperazine-1-carbonyl)benzyl)quinazoline-2,4(1H,3H)-dione;
1-(4-(4-Benzoylpiperazine-1-carbonyl)benzyl)quinazoline-2,4(1H,3H)-dione;
1-(4-(4-(Pyridin-2-yl)piperazine-1-carbonyl)benzyl)quinazoline-2,4(1H,3H)-dione;
1-(4-(4-(4-Methoxybenzoyl)piperazine-1-carbonyl)benzyl)quinazoline-2,4(1H,3H)-dione;
1-(4-(4-(4-Fluorobenzoyl)piperazine-1-carbonyl)benzyl)quinazoline-2,4(1H,3H)-dione;
1-(4-(4-(Pyrimidin-2-yl)piperazine-1-carbonyl)benzyl)quinazoline-2,4(1H,3H)-dione;
1-(4-(4-(4-Bromobenzoyl)piperazine-1-carbonyl)benzyl)quinazoline-2,4(1H,3H)-dione;
1-(4-(4-(Cyclopentylcarbonyl)piperazine-1-carbonyl)benzyl)quinazoline-2,4(1H,3H)-dione;
5-Chloro-1-(3-(4-(pyrimidin-2-yl)piperazine-1-carbonyl)benzyl)quinazoline-2,4(1H,3H)-dione;
5-Chloro-1-(3-(4-(cyclopentylcarbonyl)piperazine-1-carbonyl)benzyl)quinazoline-2,4(1H,3H)-dione;
5-Chloro-1-(3-(4-(cyclohexylcarbonyl)piperazine-1-carbonyl)benzyl)quinazoline-2,4(1H, 3H)-dione;
5-Chloro-1-(4-fluoro-3-(4-(pyrimidin-2-yl)piperazine-1-carbonyl)benzyl)quinazoline-2,4(1H,3H)-dione;
5-Chloro-1-(4-fluoro-3-(4-(cyclopentylcarbonyl)piperazine-1-carbonyl)benzyl)quinazoline-2,4(1H,3H)-dione;
5-Chloro-1-(4-fluoro-3-(4-(cyclohexylcarbonyl)piperazine-1-carbonyl)benzyl)quinazoline-2,4(1H,3H)-dione;
5-Chloro-1-(4-fluoro-3-(4-(pyridin-2-yl)piperazine-1-carbonyl)benzyl)quinazoline-2,4(1H,3H)-dione;
5-Chloro-1-(4-fluoro-3-(4-(thiophene-2-carbonyl)piperazine-1-carbonyl)benzyl) quinazoline-2,4(1H,3H)-dione;
5-Chloro-1-(6-fluoro-3-(4-(cyclohexylcarbonyl)piperazine-1-carbonyl)benzyl)quinazoline-2,4(1H,3H)-dione;
5-Chloro-1-(6-fluoro-3-(4-(cyclopentylcarbonyl)piperazine-1-carbonyl)benzyl)quinazoline-2,4(1H,3H)-dione;
5-Chloro-1-(6-fluoro-3-(4-(pyrimidin-2-yl)piperazine-1-carbonyl)benzyl)quinazoline-2,4(1H,3H)-dione;
6-Chloro-1-(3-(4-(pyrimidin-2-yl)piperazine-1-carbonyl)benzyl)quinazoline-2,4(1H,3H)-dione;
6-Chloro-1-(3-(4-(cyclopentylcarbonyl)piperazine-1-carbonyl)benzyl)quinazoline-2,4(1H,3H)-dione;
6-Chloro-1-(3-(4-(cyclohexylcarbonyl)piperazine-1-carbonyl)benzyl)quinazoline-2,4(1H, 3H)-dione;
6-Chloro-1-(3-(4-(pyridin-2-yl)piperazine-1-carbonyl)benzyl)quinazoline-2,4(1H,3H)-dione;
6-Chloro-1-(3-(4-(thiophene-2-carbonyl)piperazine-1-carbonyl)benzyl)quinazoline-2,4(1H,3H)-dione;
6-Chloro-1-(3-(4-(cyclobutylcarbonyl)piperazine-1-carbonyl)benzyl)quinazoline-2,4(1H, 3H)-dione;
6-Chloro-1-(4-fluoro-3-(4-(pyrimidin-2-yl)piperazine-1-carbonyl)benzyl)quinazoline-2,4(1H,3H)-dione;
6-Chloro-1-(3-(4-(cyclopentylcarbonyl)piperazine-1-carbonyl)-4-fluorobenzyl)quinazoline-2,4(1H,3H)-dione;
6-Chloro-1-(3-(4-(cyclohexylcarbonyl)piperazine-1-carbonyl)-4-fluorobenzyl)quinazoline-2,4(1H,3H)-dione;
6-Chloro-1-(4-fluoro-3-(4-(pyridin-2-yl)piperazine-1-carbonyl)benzyl)quinazoline-2,4(1H,3H)-dione;
6-Chloro-1-(4-fluoro-3-(4-(thiophene-2-carbonyl)piperazine-1-carbonyl)benzyl) quinazoline-2,4(1H,3H)-dione;
6-Chloro-1-(4-fluoro-3-(4-(cyclopropylcarbonyl)piperazine-1-carbonyl)benzyl)quinazoline)-2,4(1H,3H)-dione;
6-Chloro-1-(6-fluoro-3-(4-(pyrimidin-2-yl)piperazine-1-carbonyl)benzyl)quinazoline-2,4(1H,3H)-dione;
6-Chloro-1-(6-fluoro-3-(4-(cyclopentylcarbonyl)piperazine-1-carbonyl)benzyl)quinazoline-2,4(1H,3H)-dione;
6-Chloro-1-(6-fluoro-3-(4-(cyclohexylcarbonyl)piperazine-1-carbonyl)benzyl)quinazoline-2,4(1H,3H)-dione;
6-Chloro-1-(6-fluoro-3-(4-(thiophene-2-carbonyl)piperazine-1-carbonyl)benzyl) quinazoline-2,4(1H,3H)-dione;
5-Fluoro-1-(3-(4-(pyrimidin-2-yl)piperazine-1-carbonyl)benzyl)quinazoline-2,4(1H,3H)-dione;
5-Fluoro-1-(3-(4-(Cyclopentylcarbonyl)piperazine-1-carbonyl)benzyl)quinazoline-2,4(1H,3H)-dione;
5-Fluoro-1-(3-(4-(Cyclohexylcarbonyl)piperazine-1-carbonyl)benzyl)quinazoline-2,4(1H, 3H)-dione;
5-Fluoro-1-(3-(4-(pyridin-2-yl)piperazine-1-carbonyl)benzyl)quinazoline-2,4(1H,3H)-dione;
5-Fluoro-(1-(3-(4-(thiophene-2-carbonyl)piperazine-1-carbonyl)benzyl)quinazoline)-2,4 (1H,3H)-dione;
5-Fluoro-1-(4-fluoro-3-(4-(pyrimidin-2-yl)piperazine-1-carbonyl)benzyl)quinazoline-2,4 (1H,3H)-dione;
5-Fluoro-1-(4-fluoro-3-(4-(cyclopentylcarbonyl)piperazine-1-carbonyl)benzyl)quinazoline-2,4(1H,3H)-dione;
5-Fluoro-1-(4-fluoro-3-(4-(cyclohexylcarbonyl)piperazine-1-carbonyl)benzyl)quinazoline-2,4(1H,3H)-dione;
5-Fluoro-1-(4-fluoro-3-(4-(pyridin-2-yl)piperazine-1-carbonyl)benzyl)quinazoline-2,4(1H,3H)-dione;
5-Fluoro-1-(4-fluoro-3-(4-(thiophene-2-carbonyl)piperazine-1-carbonyl)benzyl) quinazoline-2,4(1H,3H)-dione;
5-Fluoro-1-(4-fluoro-3-(4-Benzoylpiperazine-1-carbonyl)benzyl)quinazoline-2,4(1H,3H)-dione;
8-Fluoro-1-(3-(4-(cyclohexylcarbonyl)piperazine-1-carbonyl)benzyl)quinazoline-2,4(1H, 3H)-dione;
6-Fluoro-1-(3-(4-(pyrimidin-2-yl)piperazine-1-carbonyl)benzyl)quinazoline-2,4(1H,3H)-dione;
6-Fluoro-1-(3-(4-(pyridin-2-yl)piperazine-1-carbonyl)benzyl)quinazoline-2,4(1H,3H)-dione;
6-Fluoro-(1-(3-(4-(thiophene-2-carbonyl)piperazine-1-carbonyl)benzyl)quinazoline)-2,4 (1H,3H)-dione;
6-Fluoro-1-(3-(4-(cyclohexylcarbonyl)piperazine-1-carbonyl)benzyl)quinazoline-2,4(1H, 3H)-dione;
6-Fluoro-1-(3-(4-(cyclopentylcarbonyl)piperazine-1-carbonyl)benzyl)quinazoline-2,4(1H,3H)-dione;
6-Fluoro-1-(4-fluoro-3-(4-(pyrimidin-2-yl)piperazine-1-carbonyl)benzyl)quinazoline-2,4 (1H,3H)-dione;
6-Fluoro-1-(4-fluoro-3-(4-(pyridin-2-yl)piperazine-1-carbonyl)benzyl)quinazoline-2,4(1H,3H)-dione;
6-Fluoro-1-(4-fluoro-3-(4-(thiophene-2-carbonyl)piperazine-1-carbonyl)benzyl) quinazoline-2,4(1H,3H)-dione;
6-Fluoro-1-(4-fluoro-3-(4-(cyclohexylcarbonyl)piperazine-1-carbonyl)benzyl)quinazoline-2,4(1H,3H)-dione;
6-Fluoro-1-(4-fluoro-3-(4-(cyclopentylcarbonyl)piperazine-1-carbonyl)benzyl)quinazoline-2,4(1H,3H)-dione;
6-Fluoro-1-(6-fluoro-3-(4-(pyrimidin-2-yl)piperazine-1-carbonyl)benzyl)quinazoline-2,4 (1H,3H)-dione;
6-Fluoro-1-(6-fluoro-3-(4-(pyridin-2-yl)piperazine-1-carbonyl)benzyl)quinazoline-2,4(1H,3H)-dione;
6-Fluoro-1-(6-fluoro-3-(4-(thiophene-2-carbonyl)piperazine-1-carbonyl)benzyl) quinazoline-2,4(1H,3H)-dione;
6-Fluoro-1-(6-fluoro-3-(4-(cyclohexylcarbonyl)piperazine-1-carbonyl)benzyl)quinazoline-2,4(1H,3H)-dione;
6-Fluoro-1-(6-fluoro-3-(4-(cyclopentylcarbonyl)piperazine-1-carbonyl)benzyl)quinazoline-2,4(1H,3H)-dione;
5-Fluoro-1-(6-fluoro-3-(4-(pyrimidin-2-yl)piperazine-1-carbonyl)benzyl)quinazoline-2,4 (1H,3H)-dione;
5-Fluoro-1-(6-fluoro-3-(4-(pyridin-2-yl)piperazine-1-carbonyl)benzyl)quinazoline-2,4(1H,3H)-dione;
5-Fluoro-1-(6-fluoro-3-(4-(thiophene-2-carbonyl)piperazine-1-carbonyl)benzyl) quinazoline-2,4(1H,3H)-dione;
5-Fluoro-1-(6-fluoro-3-(4-(cyclohexylcarbonyl)piperazine-1-carbonyl)benzyl)quinazoline-2,4(1H,3H)-dione;
5-Fluoro-1-(6-fluoro-3-(4-(cyclopentylcarbonyl)piperazine-1-carbonyl)benzyl)quinazoline-2,4(1H,3H)-dione;
5-Methyl-1-(3-(4-(pyrimidin-2-yl)piperazine-1-carbonyl)benzyl)quinazoline-2,4(1H,3H)-dione;
5-Methyl-1-(3-(4-(cyclopentylcarbonyl)piperazine-1-carbonyl)benzyl)quinazoline-2,4(1H,3H)-dione;
7-Chloro-1-(3-(4-(pyrimidin-2-yl)piperazine-1-carbonyl)benzyl)quinazoline-2,4(1H,3H)-dione;
7-Chloro-1-(3-(4-(cyclopentylcarbonyl)piperazine-1-carbonyl)benzyl)quinazoline-2,4(1H,3H)-dione;
5-Fluoro-1-(4-fluoro-3-(4-(cyclopropylcarbonyl)piperazine-1-carbonyl)benzyl)quinazoline)-2,4(1H,3H)-dione;
6-Chloro-(4-fluoro-1-(3-(4-benzoylpiperazine-1-carbonyl)benzyl)quinazoline)-2,4(1H,3H)-dione;
5-Fluoro-1-(3-(4-(cyclobutylcarbonyl)piperazine-1-carbonyl)benzyl)quinazoline-2,4(1H, 3H)-dione;
5-fluoro-1-(4-fluoro-3-(4-(cyclobutylcarbonyl)piperazine-1-carbonyl)benzyl)quinazoline-2,4(1H,3H)-dione;
6-Chloro-1-(6-fluoro-3-(4-(cyclobutylcarbonyl)piperazine-1-carbonyl)benzyl)quinazoline-2,4(1H,3H)-dione;
5-Chloro-1-(4-fluoro-3-(4-(cyclobutylcarbonyl)piperazine-1-carbonyl)benzyl)quinazoline-2,4(1H,3H)-dione;
6-Chloro-1-(4-fluoro-3-(4-(cyclobutylcarbonyl)piperazine-1-carbonyl)benzyl)quinazoline-2,4(1H,3H)-dione;
5-Chloro-1-(6-fluoro-3-(4-(cyclobutylcarbonyl)piperazine-1-carbonyl)benzyl)quinazoline-2,4(1H,3H)-dione;
5-Chloro-1-(6-fluoro-3-(4-(thiophene-2-carbonyl)piperazine-1-carbonyl)benzyl) quinazoline-2,4(1H,3H)-dione;
5-Fluoro-1-(4-fluoro-3-(4-(furan-2-carbonyl)piperazine-1-carbonyl)benzyl)quinazoline-2,4(1H,3H)-dione;
5-Chloro-1-(6-fluoro-3-(4-(pyridin-2-yl)piperazine-1-carbonyl)benzyl)quinazoline-2,4(1H,3H)-dione;
6-Chloro-1-(6-fluoro-3-(4-(pyridin-2-yl)piperazine-1-carbonyl)benzyl)quinazoline-2,4(1H,3H)-dione;
6-Chloro-1-(5-fluoro-3-(4-(pyrimidin-2-yl)piperazine-1-carbonyl)benzyl)quinazoline-2,4(1H,3H)-dione;
5-Fluoro-1-(5-fluoro-3-(4-(pyrimidin-2-yl)piperazine-1-carbonyl)benzyl)quinazoline-2,4 (1H,3H)-dione;
1-(3-(4-Cyclopentylpiperazine-1-carbonyl)benzyl)quinazoline-2,4(1H,3H)-dione;
6-Fluoro-1-(4-fluoro-3-(4-(cyclopropylcarbonyl)piperazine-1-carbonyl)benzyl)quinazoline-2,4(1H,3H)-dione;
6-Fluoro-1-(3-(4-(cyclobutylcarbonyl)piperazine-1-carbonyl)benzyl)quinazoline-2,4(1H, 3H)-dione;
6-Fluoro-1-(4-fluoro-3-(4-(cyclobutylcarbonyl)piperazine-1-carbonyl)benzyl)quinazoline-2,4(1H,3H)-dione;
6-Fluoro-1-(4-fluoro-3-(4-benzoylpiperazine-1-carbonyl)benzyl)quinazoline-2,4(1H,3H)-dione;
5-Fluoro-1-(6-fluoro-3-(4-(cyclobutylcarbonyl)piperazine-1-carbonyl)benzyl)quinazoline-2,4(1H,3H)-dione;
6-Fluoro-1-(6-fluoro-3-(4-(cyclobutylcarbonyl)piperazine-1-carbonyl)benzyl)quinazoline-2,4(1H,3H)-dione;
5-Fluoro-1-(6-fluoro-3-(4-(furan-2-carbonyl)piperazine-1-carbonyl)benzyl)quinazoline-2,4(1H,3H)-dione;
6-Fluoro-1-(4-fluoro-3-(4-(furan-2-carbonyl)piperazine-1-carbonyl)benzyl)quinazoline-2,4(1H,3H)-dione;
6-Fluoro-1-(5-fluoro-3-(4-(pyrimidin-2-yl)piperazine-1-carbonyl)benzyl)quinazoline-2,4 (1H,3H)-dione;
6-Fluoro-1-(6-chloro-3-(4-(pyrimidin-2-yl)piperazine-1-carbonyl)benzyl)quinazoline-2,4(1H,3H)-dione;
6-Fluoro-1-(4-fluoro-3-(4-(thiazol-2-yl)piperazine-1-carbonyl)benzyl)quinazoline-2,4(1H,3H)-dione;
5-Fluoro-1-(4-fluoro-3-(4-(thiazol-2-yl)piperazine-1-carbonyl)benzyl)quinazoline-2,4(1H,3H)-dione;
1-(4-Fluoro-3-(4-(thiazol-2-yl)piperazine-1-carbonyl)benzyl)quinazoline-2,4(1H,3H)-dione;
1-(3-(4-(Cyclohexylcarbamoyl)piperazine-1-carbonyl)benzyl)quinazoline-2,4(1H,3H)-dione;
1-(4-Fluoro-3-(4-(pyrimidin-2-yl)piperazine-1-carbonyl)benzyl)quinazoline-2,4(1H,3H)-dione hydrochloride;
5-Fluoro-1-(4-fluoro-3-(4-(pyrimidin-2-yl)piperazine-1-carbonyl)benzyl)quinazoline-2,4 (1H,3H)-dione hydrochloride;
1-(3-(4-(Pyrimidin-2-yl)piperazine-1-carbonyl)benzyl)quinazoline-2,4(1H,3H)-dione hydrochloride;
1-(3-((4-(Pyridin-2-yl)piperazin-1-yl)methyl)benzyl)quinazoline-2,4(1H,3H)-dione;
1-(3-(Naphthalen-2-yl)acetamidobenzyl)quinazoline-2,4(1H,3H)-dione;
1-(3-(3,4-Dimethoxyphenyl)acetamidobenzyl)quinazoline-2,4(1H,3H)-dione;
1-(3-([1,2,4]Triazolo[4,3-a]pyridine-6-carbonyl)benzyl)quinazoline-2,4(1H,3H)-dione;
6-Fluoro-1-((2-(4-(pyrimidin-2-yl)piperazine-1-carbonyl)pyridin-6-yl)methyl)quinazoline-2,4(1H,3H)-dione;
5-Fluoro-1-(4-fluoro-3-(4-methoxybenzylcarbamoyl)benzyl)quinazoline-2,4(1H,3H)-dione;
5-Fluoro-1-(4-fluoro-3-(3-chlorobenzylcarbamoyl)benzyl)quinazoline-2,4(1H,3H)-dione;
5-Fluoro-1-(4-fluoro-3-(benzylcarbamoyl)benzyl)quinazoline-2,4(1H,3H)-dione;
5-Methoxy-1-(3-(4-(pyrimidin-2-yl)piperazine-1-carbonyl)benzyl)quinazoline-2,4(1H,3H)-dione;
6-Methoxy-1-(3-(4-(pyrimidin-2-yl)piperazine-1-carbonyl)benzyl)quinazoline-2,4(1H,3H)-dione;
5-Fluoro-1-(4-fluoro-3-(5-bromopyrimidin-2-aminocarbamoyl)benzyl)quinazoline-2,4(1H,3H)-dione;
7-Trifluoromethyl-1-(3-(4-(Pyrimidin-2-yl)piperazine-1-carbonyl)benzyl)quinazoline-2,4(1H,3H)-dione;
6,7-Ethylenedioxo-1-(3-(4-(pyrimidin-2-yl)piperazine-1-carbonyl)benzyl)quinazoline-2,4(1H,3H)-dione;
5-Fluoro-1-(6-methoxy-3-(4-(pyrimidin-2-yl)piperazine-1-carbonyl)benzyl)quinazoline-2,4(1H,3H)-dione;
7-Methoxy-1-(3-(4-(pyrimidin-2-yl)piperazine-1-carbonyl)benzyl)quinazoline-2,4(1H,3H)-dione;
5-Fluoro-1-(4-fluoro-3-(4-(tetrahydrofuran-2-carbonyl)piperazine-1-carbonyl)benzyl) quinazoline-2,4(1H,3H)-dione;
5-Fluoro-1-(4-nitro-3-(4-(pyrimidin-2-yl)piperazine-1-carbonyl)benzyl)quinazoline-2,4 (1H,3H)-dione;
5-Fluoro-1-(4-fluoro-3-(4-cyclohexylpiperazine-1-carbonyl)benzyl)quinazoline-2,4(1H,3H)-dione;
5-Fluoro-1-(4-fluoro-3-(4-phenylpiperazine-1-carbonyl)benzyl)quinazoline-2,4(1H,3H)-dione;
5-Fluoro-1-(4-fluoro-3-(4-phenylpiperidine-1-carbonyl)benzyl)quinazoline-2,4(1H,3H)-dione;
5-Fluoro-1-(4-bromo-3-(4-(pyrimidin-2-yl)piperazine-1-carbonyl)benzyl)quinazoline-2,4(1H,3H)-dione;
6,7-Methylenedioxo-1-(3-(4-(pyrimidin-2-yl)piperazine-1-carbonyl)benzyl)quinazoline-2,4(1H,3H)-dione;
5-Fluoro-1-(4-fluoro-3-(4-(cyclohexylmethyl)piperazine-1-carbonyl)benzyl)quinazoline-2,4(1H,3H)-dione;
8-Fluoro-1-(3-(4-(pyrimidin-2-yl)piperazine-1-carbonyl)benzyl)quinazoline-2,4(1H,3H)-dione;
6-Amino-1-(3-(4-(pyrimidin-2-yl)piperazine-1-carbonyl)benzyl)quinazoline-2,4(1H,3H)-dione;
1-(2-(4-(Pyrimidin-2-yl)piperazine-1-carbonyl)benzyl)quinazoline-2,4(1H,3H)-dione;
8-Chloro-1-(3-(4-(pyrimidin-2-yl)piperazine-1-carbonyl)benzyl)quinazoline-2,4(1H,3H)-dione;
8-Methyl-1-(3-(4-(pyrimidin-2-yl)piperazine-1-carbonyl)benzyl)quinazoline-2,4(1H,3H)-dione;
8-Methoxy-1-(3-(4-(pyrimidin-2-yl)piperazine-1-carbonyl)benzyl)quinazoline-2,4(1H,3H)-dione; and
8-Hydroxy-1-(3-(4-(pyrimidin-2-yl)piperazine-1-carbonyl)benzyl)quinazoline-2,4(1H,3H)-dione;
or pharmaceutically acceptable salts, solvates or prodrugs thereof.

6. The method of claim 1, wherein the DNA damaging anti-cancer drug is an alkylating agent anti-cancer drug selected from the group consisting of: nitrogen mustard N-oxide, cyclophosphamide, ifosfamide, thiotepa, ranimustine, nimustine, Temozolomide (TMZ), altretamine, apaziquone, brostallicin, bendamustine, carmustine, estramustine, fotemustine, glufosfamide, mafosfamide, bendamustine, dibromidulcitol, cisplatin, carboplatin, eptaplatin, lobaplatin, nedaplatin, oxaliplatin and satraplatin.

7. The method of claim 1, wherein the method comprises administering to the subject one or more pharmaceutical compositions of the PARP inhibitor in a daily dose of 20-120 mg or 20-100 mg, and one or more pharmaceutical compositions of the DNA damaging anti-cancer drug in a daily dose that is about 1/12th to about ⅕th of the daily dose of the DNA damaging anti-cancer drug when used alone or in combination with other drugs or therapies, or that is 10-30 mg.

8. The method of claim 1, wherein the method comprises administering to the subject one or more pharmaceutical compositions of the PARP inhibitor in a daily dose of 20-120 mg, or 20-100 mg or 40-80 mg, or 20, 40, 60, 80 or 100 mg, and one or more pharmaceutical compositions of the DNA damaging anti-cancer drug in a daily dose of 10-30 mg, or 10 mg, 20 mg or 30 mg, for at least 7 consecutive days, or for at least one cycle, wherein one cycle includes 28 consecutive days of administration of the PARP inhibitor and 21 consecutive days of administration of the DNA damaging anti-cancer drug.

9. The method of claim 7, wherein the method comprises administering 5-fluoro-1-(4-fluoro-3-(4-pyrimidin-2-yl)piperazine-1-carbonyl)benzyl)quinazoline-2,4(1H,3H)-dione once a day in a daily dose of 20, 40, 60 or 80 mg for 28 consecutive days and TMZ once a day in a daily dose of 10, 20 or 30 mg for 21 consecutive days, for a 28-day cycle.

10. The method of claim 1, wherein the cancer is selected from the group consisting of liver cancer, melanoma, Hodgkin's disease, non-Hodgkin's lymphoma, acute lymphocytic leukemia, chronic lymphocytic leukemia, multiple myeloma, neuroblastoma, breast cancer, ovarian cancer, non-small cell lung cancer, small cell lung cancer, Wilms tumor, cervical cancer, testicular cancer, soft tissue sarcoma, primary macroglobulinemia, bladder cancer, chronic myeloid leukemia, primary brain cancer, malignant melanoma, gastric cancer, colon cancer, malignant pancreatic islet tumor, malignant carcinoid cancer, choriocarcinoma, mycosis fungoides, head and neck cancer, osteogenic sarcoma, pancreatic cancer, acute myeloid leukemia, hairy cell leukemia, rhabdomyosarcoma, Kaposi's sarcoma, urogenital tumors, thyroid cancer, esophageal cancer, malignant hypercalcemia, cervical hyperplasia, renal cell carcinoma, endometrial cancer, polycythemia vera, idiopathic thrombocythemia, adrenocortical carcinoma, skin cancer and prostate cancer.

11. A kit containing one or more pharmaceutical preparations of a PARP inhibitor and one or more pharmaceutical preparations of a DNA damaging anti-cancer drug, wherein the pharmaceutical preparations of the PARP inhibitor and the pharmaceutical preparations of the DNA damaging anti-cancer drug are individually packaged, wherein in the pharmaceutical preparation of PARP inhibitor, the content of the PARP inhibitor is able to provide an effective daily dose of the PARP inhibitor when being administered alone, and in the pharmaceutical preparation of the DNA damaging anti-cancer drug, the content of the DNA damaging anti-cancer drug is able to provide a daily dose that is about 1/12th to about ⅕th of the daily dose of the DNA damaging anti-cancer drug when used alone or in combination with other drugs or therapies; wherein the PARP inhibitor is selected from the group consisting of compounds represented by Formula I: 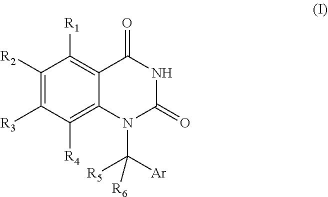
or pharmaceutically acceptable salts, solvates or prodrugs thereof, wherein:
Ar is an optionally substituted aryl or an optionally substituted heteroaryl; and
R₁-R₆ are independently hydrogen, halo, optionally substituted amino, optionally substituted alkoxy, optionally substituted C₁₋₁₀ alkyl, alkenyl, alkynyl, nitro, cyano, acylamido, hydroxy, thiol, acyloxy, azido, carboxy, ethylenedioxo, hydroxylamido or optionally substituted alkylthiol;
wherein the DNA damaging anti-cancer drug is an alkylating agent anti-cancer drug selected from the group consisting of nitrogen mustard N-oxide, cyclophosphamide, ifosfamide, thiotepa, ranimustine, nimustine, Temozolomide (TMZ), altretamine, apaziquone, brostallicin, bendamustine, carmustine, estramustine, fotemustine, glufosfamide, mafosfamide, bendamustine, dibromidulcitol, cisplatin, carboplatin, eptaplatin, lobaplatin, nedaplatin, oxaliplatin and satraplatin.

12. The kit of claim 11, wherein the kit contains one or more pharmaceutical preparations of 5-fluoro-1-(4-fluoro-3-(4-(pyrimidin-2-yl)piperazine-1-carbonyl)benzyl)quinazoline-2,4(1H, 3H)-dione and one or more pharmaceutical preparations of Temozolomide, wherein in the pharmaceutical preparations of 5-fluoro-1-(4-fluoro-3-(4-(pyrimidin-2-yl)piperazine-1-carbonyl)benzyl)quinazoline-2,4(1H,3H)-dione, the content of 5-fluoro-1-(4-fluoro-3-(4-(pyrimidin-2-yl)piperazine-1-carbonyl)benzyl)quinazoline-2,4(1H,3H)-dione meets a daily dose requirement of 20-120 mg, and in the pharmaceutical preparations of Temozolomide, the content of Temozolomide meets a daily dose requirement of 10-30 mg.

13. The kit of claim 10, wherein the kit contains one or more pharmaceutical preparations of 5-fluoro-1-(4-fluoro-3-(4-(pyrimidin-2-yl)piperazine-1-carbonyl)benzyl)quinazoline-2,4(1H,3H)-dione and one or more pharmaceutical preparations of TMZ, to provide;
5-fluoro-1-(4-fluoro-3-(4-(pyrimidin-2-yl)piperazine-1-carbonyl)benzyl)quinazoline-2,4(1H, 3H-dione in a daily dose of 40 mg and TMZ in a daily dose of 20 mg, or
5-fluoro-1-(4-fluoro-3-(4-(pyrimidin-2-yl)piperazine-1-carbonyl)benzyl)quinazoline-2,4(1H, 3H-dione in a daily dose of 60 mg and TMZ in a daily dose of 20 mg, or
5-fluoro-1-(4-fluoro-3-(4-(pyrimidin-2-yl)piperazine-1-carbonyl)benzyl)quinazoline-2,4(1H, 3H)-dione in a daily dose of 80 mg and TMZ in a daily dose of 20 mg, or
5-fluoro-1-(4-fluoro-3-(4-(pyrimidin-2-yl)piperazine-1-carbonyl)benzyl)quinazoline-2,4(1H, 3H-dione in a daily dose of 80 mg and TMZ in a daily dose of 30 mg, or
5-fluoro-1-(4-fluoro-3-(4-(pyrimidin-2-yl)piperazine-1-carbonyl)benzyl)quinazoline-2,4(1H, 3H-dione in a daily dose of 40 mg and TMZ in a daily dose of 10 mg.

14. A compound preparation, containing 5-fluoro-1-(4-fluoro-3-(4-(pyrimidin-2-yl)piperazine-1-carbonyl)benzyl)quinazoline-2,4(1H,3H)-dione and Temozolomide, wherein in the compound preparation, the content of 5-fluoro-1-(4-fluoro-3-(4-(pyrimidin-2-yl)piperazine-1-carbonyl)benzyl)quinazoline-2,4(1H,3H)-dione meets a daily dose requirement of 20-120 mg, and the content of Temozolomide meets a daily dose requirement of 10-30 mg.

15. (canceled)

16. The kit of claim 11, wherein the PARP inhibitor is selected from the group consisting of compounds represented by Formula II: 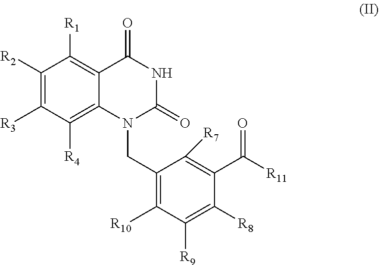
or pharmaceutically acceptable salts, solvates or prodrugs thereof wherein:
R₁-R₄ are independently hydrogen, halo, optionally substituted amino, optionally substituted alkoxy, optionally substituted C₁₋₁₀ alkyl, alkenyl, alkynyl, nitro, cyano, acylamido, hydroxy, thiol, acyloxy, azido, carboxy, ethylenedioxo, hydroxylamido or optionally substituted alkylthiol;
R₇-R₁₀ are independently hydrogen, halo, optionally substituted amino, alkoxy, C₁₋₁₀ alkyl, haloalkyl, aryl, heteroaryl, carbocyclic group, heterocyclic group, alkenyl, alkynyl, arylalkyl, arylalkenyl, arylalkynyl, heteroarylalkyl, heteroarylalkenyl, heteroarylalkynyl, carbocycloalkyl, heterocycloalkyl, hydroxyalkyl, hydroxyalkoxy, aminoalkyl, aminoalkoxy, carboxyalkyl, carboxyalkoxy, nitro, cyano, acylamido, aminocarbonyl, hydroxy, thiol, acyloxy, azido, carboxy, hydroxylamido, alkylsulfonyl, aminosulfonyl, di-substituted alkylaminosulfonyl, alkylsulfiniyl, alkylthiol, or substituted carbonyl; and
R₁₁ is an optionally substituted amino, hydrazine, alkoxy, C₁₋₁₀ alkyl, haloalkyl, aryl, heteroaryl, carbocyclic group, heterocyclic group, alkenyl, alkynyl, arylalkyl, arylalkenyl, arylalkynyl, heteroarylalkyl, heteroarylalkenyl, heteroarylalkynyl, carbocycloalkyl, heterocycloalkyl, hydroxyalkyl, hydroxyalkoxy, aminoalkyl, aminoalkoxy, carboxyalkyl, carboxyalkoxy, acylamido, hydroxy, thiol, acyloxy, hydroxylamido, or alkylthiol.

17. The kit of claim 11, wherein the PARP inhibitor is selected from the group consisting of compounds represented by Formula III: 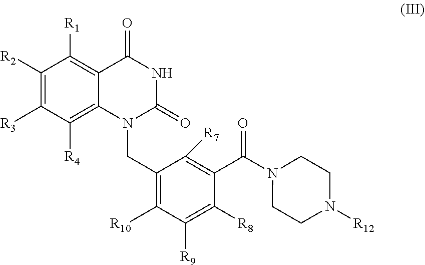
or pharmaceutically acceptable salts, solvates or prodrugs thereof, wherein:
R₁-R₄ are independently hydrogen, halo, optionally substituted amino, optionally substituted alkoxy, optionally substituted C₁₋₁₀ alkyl, alkenyl, alkynyl, nitro, cyano, acylamido, hydroxy, thiol, acyloxy, azido, carboxy, ethylenedioxo, hydroxylamido or optionally substituted alkylthiol; R₇-R₁₀ are independently hydrogen, halo, optionally substituted amino, alkoxy, C₁₋₁₀ alkyl, haloalkyl, aryl, heteroaryl, a carbocyclic group, a heterocyclic group, alkenyl, alkynyl, arylalkyl, arylalkenyl, arylalkynyl, heteroarylalkyl, heteroarylalkenyl, heteroarylalkynyl, carbocycloalkyl, heterocycloalkyl, hydroxyalkyl, hydroxyalkoxy, aminoalkyl, aminoalkoxy, carboxyalkyl, carboxyalkoxy, nitro, cyano, acylamido, aminocarbonyl, hydroxy, thiol, acyloxy, azido, carboxy, hydroxylamido, alkylsulfonyl, aminosulfonyl, di-substituted alkylaminosulfonyl, alkylsulfiniyl, alkylthiol, or substituted carbonyl; and
R₁₂ is an optionally substituted C₁₋₁₀ alkyl, cycloalkyl, aryl, heteroaryl, carbocyclic group, heterocyclic group, alkenyl, alkynyl, acyl, arylalkenyl, arylalkynyl, heteroarylalkenyl, heteroarylalkynyl, alkylcarbonyl, cycloalkylcarbonyl, arylcarbonyl, heteroarylcarbonyl, heterocyclocarbonyl, aminocarbonyl, alkylsulfonyl, cycloalkylsulfonyl or aminosulfonyl.

18. The kit of claim 17, wherein,
R₁ and R₂ are independently selected from the group consisting of H, halo, C₁₋₆ alkyl and C₁₋₆ alkoxy;
R₃ is H;
R₄ is selected from the group consisting of H, halo, C₁₋₆ alkyl, C₁₋₆ alkoxy and hydroxy;
R₇, R₈, R₉ and R₁₀ are independently hydrogen or halo; and
R₁₂ is an optionally substituted cycloalkyl, aryl, heteroaryl, carbocyclic group, heterocyclic group, arylalkyl, heteroarylalkyl, carbocycloalkyl, heterocycloalkyl, alkylcarbonyl, cycloalkylcarbonyl, arylcarbonyl, heteroarylcarbonyl or heterocyclocarbonyl; or
R₁ and R₂ are independently selected from the group consisting of hydrogen, fluoro, chloro, bromo and methyl;
R₄ is hydrogen, fluoro, methoxy or hydroxyl;
R₇, R₈, R₉ and R₁₀ are independently hydrogen or halo; and
R₁₂ is an optionally substituted C₃₋₈ cycloalkyl, pyridyl, pyrimidinyl, benzoyl, phenyl, piperidinyl, thienylcarbonyl, furylcarbonyl, piperazinyl or thiazolyl; or
R₁ is halo, NH₂, C₁₋₆ alkyl, nitro or hydroxy, and R₂-R₄ are independently hydrogen, halo, NH₂, C₁₋₆ alkyl, nitro or hydroxy; or R₂ is halo, NH₂, C₁₋₆ alkyl, nitro or hydroxy, and R₁ and R₃-R₄ are independently hydrogen, halo, NH₂, C₁₋₆ alkyl, nitro or hydroxy; or R₃ is halo, NH₂, C₁₋₆ alkyl, nitro or hydroxyl, and R₁, R₂ and R₄ are independently hydrogen, halo, NH₂, C₁₋₆ alkyl, nitro or hydroxy; or R₄ is halo, NH₂, C₁₋₆ alkyl, nitro or hydroxyl, and R₁-R₃ are independently hydrogen, halo, NH₂, C₁₋₆ alkyl, nitro or hydroxyl;
R₇-R₁₀ are independently hydrogen, halo, C₁₋₆ alkoxy or nitro;
R₁₂ is pyridinyl, pyrimidinyl, C₃₋₈ cycloalkyl, C₁₋₆ alkyl optionally substituted by one C₃₋₈ cycloalkyl, benzoyl optionally substituted by one or more substituents selected from halo and C₁₋₆ alkoxy, carbonyl optionally substituted by one substituent selected from C₃₋₈ cycloalkyl, thiophenyl, pyridinyl, furanyl and tetrahydrofuranyl, C₁₋₆ alkylsulfonyl, phenyl, pyrazinyl, benzo[d]isothiazol-3-yl, benzoisoxazolyl optionally substituted by one or more halogens, and thiazolyl; or
R₁ is halo or C₁₋₆ alkyl, R₂ is hydrogen, halo, —NH₂ or C₁₋₆ alkyl, R₃ is hydrogen, halo, C₁₋₆ alkoxy or C₁₋₆ alkyl, and R₄ is hydrogen, halo, hydroxy, C₁₋₆ alkoxy or C₁₋₆ alkyl; or R₁ is hydrogen, halo or C₁₋₆ alkyl, R₂ is halo, —NH₂ or C₁₋₆ alkyl, R₃ is hydrogen, halo, C₁₋₆ alkoxy or C₁₋₆ alkyl, and R₄ is hydrogen, halo, hydroxy, C₁₋₆ alkoxy or C₁₋₆ alkyl; or R₁ is hydrogen, halo or C₁₋₆ alkyl, R₂ is hydrogen, halo, —NH₂ or C₁₋₆ alkyl, R₃ is halo, C₁₋₆ alkoxy or C₁₋₆ alkyl, and R₄ is hydrogen, halo, hydroxy, C₁₋₆ alkoxy or C₁₋₆ alkyl; or R₁ is hydrogen, halo or C₁₋₆ alkyl, R₂ is hydrogen, halo, —NH₂ or C₁₋₆ alkyl, R₃ is hydrogen, halo, C₁₋₆ alkoxy or C₁₋₆ alkyl, and R₄ is halo, hydroxy, C₁₋₆ alkoxy or C₁₋₆ alkyl;
R₇-R₁₀ are independently hydrogen, halogen, or C₁₋₆ alkyl; and
R₁₂ is C₃₋₈ cycloalkyl, C₃₋₈ cycloalkylcarbonyl, pyridyl, pyrimidinyl, benzoyl, phenyl, piperidinyl, thiophenylcarbonyl, furanyl or pyrazinyl, which is optionally substituted by 1, 2, 3, or 4 substituents selected from the group consisting of halo and C₁₋₆ alkyl; or
R₁ is halo;
R₂ is hydrogen, halo or C₁₋₆ alkyl;
R₃ is hydrogen, halo or C₁₋₆ alkyl;
R₄ is hydrogen, halo or C₁₋₆ alkyl;
R₇-R₁₀ are independently hydrogen or halo; and
R₁₂ is pyrimidinyl; or
R₁ is fluoro, chloro, bromo or methyl;
R₂ is hydrogen, fluoro, chloro, bromo or methyl;
R₃ is hydrogen, fluoro, chloro or methyl;
R₄ is hydrogen, fluoro, chloro, methoxy or methyl; and
R₇, R₈, R₉ and R₁₀ are independently hydrogen or fluoro; or
R₂ is fluoro, chloro, bromo or methyl;
R₁ is hydrogen, fluoro, chloro, bromo or methyl;
R₃ is hydrogen, fluoro, chloro or methyl;
R₄ is hydrogen, fluoro, methoxy or hydroxyl; and
R₇, R₈, R₉ and R₁₀ are independently hydrogen or fluoro; or
R₄ is fluoro, methoxy or hydroxyl;
R₁ is hydrogen, fluoro, chloro, bromo or methyl;
R₂ is hydrogen, fluoro, chloro, bromo or methyl;
R₃ is hydrogen, fluoro, chloro or methyl; and
R₇, R₈, R₉ and R₁₀ are independently hydrogen or fluoro.

19. The kit of claim 11, wherein the PARP inhibitor is selected from the group consisting of:
1-(3-(4-(Pyridin-2-yl)piperazine-1-carbonyl)benzyl)quinazoline-2,4(1H,3H)-dione;
1-(3-(4-(Pyrimidin-2-yl)piperazine-1-carbonyl)benzyl)quinazoline-2,4(1H,3H)-dione;
1-(3-(4-Cyclohexylpiperazine-1-carbonyl)benzyl)quinazoline-2,4(1H,3H)-dione;
1-(3-(4-([1,2,4]Triazolo[4,3-b]pyridazin-6-yl)piperazine-1-carbonyl)benzyl)quinazoline-2,4(1H,3H)-dione;
1-(3-(4-Ethylpiperazine-1-carbonyl)benzyl)quinazoline-2,4(1H,3H)-dione;
1-(3-(4-Benzoylpiperazine-1-carbonyl)benzyl)quinazoline-2,4(1H,3H)-dione;
1-(3-(4-(4-Fluorobenzoyl)piperazine-1-carbonyl)benzyl)quinazoline-2,4(1H,3H)-dione;
1-(3-(4-(4-Chlorobenzoyl)piperazine-1-carbonyl)benzyl)quinazoline-2,4(1H,3H)-dione;
1-(3-(4-(4-Bromobenzoyl)piperazine-1-carbonyl)benzyl)quinazoline-2,4(1H,3H)-dione;
1-(3-(4-(4-Methoxybenzoyl)piperazine-1-carbonyl)benzyl)quinazoline-2,4(1H,3H)-dione;
1-(3-(4-(Tetrahydro-2H-pyran-4-yl)carbonylpiperazine-1-carbonyl)benzyl)quinazoline-2,4(1H,3H)-dione;
1-(3-(4-(Cyclopentylcarbonyl)piperazine-1-carbonyl)benzyl)quinazoline-2,4(1H,3H)-dione;
1-(3-(4-(Cyclopropylcarbonyl)piperazine-1-carbonyl)benzyl)quinazoline-2,4(1H,3H)-dione;
1-(3-(4-(Ethylsulfonyl)piperazine-1-carbonyl)benzyl)quinazoline-2,4(1H,3H)-dione;
1-(3-(4-Acetylpiperazine-1-carbonyl)benzyl)quinazoline-2,4(1H,3H)-dione;
1-(3-(4-Phenylpiperidine-1-carbonyl)benzyl)quinazoline-2,4(1H,3H)-dione;
1-(3-(4-Phenylpiperazine-1-carbonyl)benzyl)quinazoline-2,4(1H,3H)-dione;
1-(3-(4-(Pyrazin-2-yl)piperazine-1-carbonyl)benzyl)quinazoline-2,4(1H,3H)-dione;
1-(4-Fluoro-3-methoxycarbonylbenzyl)quinazoline-2,4(1H,3H)-dione;
1-(3-Carbonyl-4-fluorobenzyl)quinazoline-2,4(1H,3H)-dione;
1-(4-Fluoro-3-(4-(Cyclopentylcarbonyl)piperazine-1-carbonyl)benzyl)quinazoline-2,4(1H,3H)-dione;
1-(4-Fluoro-3-(4-(pyridin-2-yl)piperazine-1-carbonyl)benzyl)quinazoline-2,4(1H,3H)-dione;
1-(4-Fluoro-3-(4-(pyrimidin-2-yl)piperazine-1-carbonyl)benzyl)quinazoline-2,4(1H,3H)-dione;
1-(3-(4-(Cyclohexylcarbonyl)piperazine-1-carbonyl)benzyl)quinazoline-2,4(1H,3H)-dione;
1-(3-(4-(Benzo[d]isothiazol-3-yl)piperazine-1-carbonyl)benzyl)quinazoline-2,4(1H,3H)-dione;
1-(3-(4-(Piperidin-1-yl)piperidine-1-carbonyl)benzyl)quinazoline-2,4(1H,3H)-dione;
1-(3-(4-(Pyridin-4-yl)piperazine-1-carbonyl)benzyl)quinazoline-2,4(1H,3H)-dione;
1-(3-(4-(Cyclobutylcarbonyl)piperazine-1-carbonyl)benzyl)quinazoline-2,4(1H,3H)-dione;
1-(3-(4-(6-Fluorobenzo[d]isoxazol-3-yl)piperidine-1-carbonyl)benzyl)quinazoline-2,4(1H,3H)-dione;
1-(3-(4-(Thiophene-2-carbonyl)piperazine-1-carbonyl)benzyl)quinazoline-2,4(1H,3H)-dione;
1-(3-(4-(Furan-2-carbonyl)piperazine-1-carbonyl)benzyl)quinazoline-2,4(1H,3H)-dione;
1-(3-(4-(Furan-3-carbonyl)piperazine-1-carbonyl)benzyl)quinazoline-2,4(1H,3H)-dione;
1-(3-(4-(Thiophene-3-carbonyl)piperazine-1-carbonyl)benzyl)quinazoline-2,4(1H,3H)-dione;
1-(3-(4-(Pyridine-3-carbonyl)piperazine-1-carbonyl)benzyl)quinazoline-2,4(1H,3H)-dione;
1-(3-(4-(Pyridine-2-carbonyl)piperazine-1-carbonyl)benzyl)quinazoline-2,4(1H,3H)-dione;
1-(3-(4-(Pyridine-4-carbonyl)piperazine-1-carbonyl)benzyl)quinazoline-2,4(1H,3H)-dione;
1-(3-(4-Phenoxypiperidine-1-carbonyl)benzyl)quinazoline-2,4(1H,3H)-dione;
1-(4-Fluoro-3-(4-(cyclopropylcarbonyl)piperazine-1-carbonyl)benzyl)quinazoline-2,4(1H,3H)-dione;
1-(4-Fluoro-3-(4-(cyclohexylcarbonyl)piperazine-1-carbonyl)benzyl)quinazoline-2,4(1H,3H)-dione;
1-(4-Fluoro-3-(4-(pyrazin-2-yl)piperazine-1-carbonyl)benzyl)quinazoline-2,4(1H,3H)-dione;
1-(4-Fluoro-3-(4-(benzo[d]isothiazol-3-yl)piperazine-1-carbonyl)benzyl)quinazoline-2,4(1H,3H)-dione;
1-(4-Fluoro-3-(4-(cyclobutylcarbonyl)piperazine-1-carbonyl)benzyl)quinazoline-2,4(1H,3H)-dione;
1-(4-Fluoro-3-(4-benzoylpiperazine-1-carbonyl)benzyl)quinazoline-2,4(1H,3H)-dione;
1-(4-Fluoro-3-(4-(thiophene-2-carbonyl)piperazine-1-carbonyl)benzyl)quinazoline-2,4(1H,3H)-dione;
1-(4-Fluoro-3-(4-(furan-2-carbonyl)piperazine-1-carbonyl)benzyl)quinazoline-2,4(1H,3H)-dione;
1-(6-Fluoro-3-(4-(cyclopentylcarbonyl)piperazine-1-carbonyl)benzyl)quinazoline-2,4(1H,3H)-dione;
1-(6-Fluoro-3-(4-(cyclohexylcarbonyl)piperazine-1-carbonyl)benzyl)quinazoline-2,4(1H,3H)-dione;
1-(6-Fluoro-3-(4-(pyrimidin-2-yl)piperazine-1-carbonyl)benzyl)quinazoline-2,4(1H,3H)-dione;
1-(6-Fluoro-3-(4-(pyridin-2-yl)piperazine-1-carbonyl)benzyl)quinazoline-2,4(1H,3H)-dione;
1-(6-Fluoro-3-(4-(thiophene-2-carbonyl)piperazine-1-carbonyl)benzyl)quinazoline-2,4(1H,3H)-dione;
1-(6-Fluoro-3-(4-(cyclobutylcarbonyl)piperazine-1-carbonyl)benzyl)quinazoline-2,4(1H,3H)-dione;
1-(6-Fluoro-3-(4-(furan-2-carbonyl)piperazine-1-carbonyl)benzyl)quinazoline-2,4(1H,3H)-dione;
1-(6-Chloro-3-(4-(cyclopentylcarbonyl)piperazine-1-carbonyl)benzyl)quinazoline-2,4(1H,3H)-dione;
1-(6-Chloro-3-(4-(pyrimidin-2-yl)piperazine-1-carbonyl)benzyl)quinazoline-2,4(1H,3H)-dione;
1-(6-Chloro-3-(4-(cyclohexylcarbonyl)piperazine-1-carbonyl)benzyl)quinazoline-2,4(1H,3H)-dione;
1-(6-Chloro-3-(4-(pyridin-2-yl)piperazine-1-carbonyl)benzyl)quinazoline-2,4(1H,3H)-dione;
1-(2-Chloro-3-(4-(cyclopentylcarbonyl)piperazine-1-carbonyl)benzyl)quinazoline-2,4(1H,3H)-dione;
1-(2-Chloro-3-(4-(pyrimidin-2-yl)piperazine-1-carbonyl)benzyl)quinazoline-2,4(1H,3H)-dione;
1-(2-Fluoro-3-(4-(pyrimidin-2-yl)piperazine-1-carbonyl)benzyl)quinazoline-2,4(1H,3H)-dione;
1-(2-Fluoro-3-(4-(cyclopentylcarbonyl)piperazine-1-carbonyl)benzyl)quinazoline-2,4(1H,3H)-dione;
1-(5-Fluoro-3-(4-(pyrimidin-2-yl)piperazine-1-carbonyl)benzyl)quinazoline-2,4(1H,3H)-dione;
1-(5-Fluoro-3-(4-(cyclopentylcarbonyl)piperazine-1-carbonyl)benzyl)quinazoline-2,4(1H,3H)-dione;
1-(4-Chloro-3-(4-(pyrimidin-2-yl)piperazine-1-carbonyl)benzyl)quinazoline-2,4(1H,3H)-dione;
1-(4-Chloro-3-(4-(cyclohexylcarbonyl)piperazine-1-carbonyl)benzyl)quinazoline-2,4(1H,3H)-dione;
1-((2-(4-(Cyclopentylcarbonyl)piperazine-1-carbonyl)furan-5-yl)methyl)quinazoline-2,4(1H,3H)-dione;
1-((2-(4-(Benzo[d]isothiazol-3-yl)piperazine-1-carbonyl)furan-5-yl)methyl)quinazoline-2,4(1H,3H)-dione;
1-((2-(4-(Cyclopentylcarbonyl)piperazine-1-carbonyl)pyridin-6-yl)methyl)quinazoline-2,4(1H,3H)-dione;
1-((2-(4-(Pyridin-2-yl)piperazine-1-carbonyl)pyridin-6-yl)methyl)quinazoline-2,4(1H,3H)-dione;
1-((2-(4-(Pyrimidin-2-yl)piperazine-1-carbonyl)pyridin-6-yl)methyl)quinazoline-2,4(1H,3H)-dione;
1-(3-(4-(Thiazol-2-yl)piperazine-1-carbonyl)benzyl)quinazoline-2,4(1H,3H)-dione;
1-(3-(4-(Cyclohexylmethyl)piperazine-1-carbonyl)benzyl)quinazoline-2,4(1H,3H)-dione;
1-(3-(4-(Cyclopentylmethyl)piperazine-1-carbonyl)benzyl)quinazoline-2,4(1H,3H)-dione;
1-(3-(4-(Cyclohexylsulfonyl)piperazine-1-carbonyl)benzyl)quinazoline-2,4(1H,3H)-dione;
1-(3-(4-(cyclopentylcarbonyl)piperazine-1-carbonyl)benzyl)quinazoline-2,4(1H,3H)-dione;
7-Fluoro-1-(3-(4-(pyrimidin-2-yl)piperazine-1-carbonyl)benzyl)quinazoline-2,4(1H,3H)-dione;
1-(3-(4-(cyclopentylcarbonyl)piperazine-1-carbonyl)benzyl)quinazoline-2,4(1H,3H)-dione;
7-Methyl-1-(3-(4-(pyrimidin-2-yl)piperazine-1-carbonyl)benzyl)quinazoline-2,4(1H,3H)-dione;
6-Methyl-1-(3-(4-(pyrimidin-2-yl)piperazine-1-carbonyl)benzyl)quinazoline-2,4(1H,3H)-dione;
1-(3-(4-(cyclopentylcarbonyl)piperazine-1-carbonyl)benzyl)quinazoline-2,4(1H,3H)-dione;
1-(4-Fluoro-3-(4-(pyrimidin-2-yl)piperazine-1-carbonyl)benzyl)quinazoline-2,4(1H,3H)-dione;
1-(4-fluoro-3-(4-(cyclohexylcarbonyl)piperazine-1-carbonyl)benzyl)quinazoline-2,4(1H,3H)-dione;
1-(4-fluoro-3-(4-(cyclopentylcarbonyl)piperazine-1-carbonyl)benzyl)quinazoline-2,4(1H,3H)-dione;
6-Bromo-1-(3-(4-(cyclohexylcarbonyl)piperazine-1-carbonyl)benzyl)quinazoline-2,4(1H,3H)-dione;
6-Bromo-1-(3-(4-(cyclopentylcarbonyl)piperazine-1-carbonyl)benzyl)quinazoline-2,4(1H,3H)-dione;
6-Bromo-1-(3-(4-(pyrimidin-2-yl)piperazine-1-carbonyl)benzyl)quinazoline-2,4(1H,3H)-dione;
1-(3-(4-(cyclopentylcarbonyl)piperazine-1-carbonyl)benzyl)quinazoline-2,4(1H,3H)-dione;
6-Nitro-1-(3-(4-(pyrimidin-2-yl)piperazine-1-carbonyl)benzyl)quinazoline-2,4(1H,3H)-dione;
1-(3-(4-(cyclohexylcarbonyl)piperazine-1-carbonyl)benzyl)quinazoline-2,4(1H,3H)-dione;
1-(4-(4-Benzoylpiperazine-1-carbonyl)benzyl)quinazoline-2,4(1H,3H)-dione;
1-(4-(4-(Pyridin-2-yl)piperazine-1-carbonyl)benzyl)quinazoline-2,4(1H,3H)-dione;
1-(4-(4-(4-Methoxybenzoyl)piperazine-1-carbonyl)benzyl)quinazoline-2,4(1H,3H)-dione;
1-(4-(4-(4-Fluorobenzoyl)piperazine-1-carbonyl)benzyl)quinazoline-2,4(1H,3H)-dione;
1-(4-(4-(Pyrimidin-2-yl)piperazine-1-carbonyl)benzyl)quinazoline-2,4(1H,3H)-dione;
1-(4-(4-(4-Bromobenzoyl)piperazine-1-carbonyl)benzyl)quinazoline-2,4(1H,3H)-dione;
1-(4-(4-(Cyclopentylcarbonyl)piperazine-1-carbonyl)benzyl)quinazoline-2,4(1H,3H)-dione;
5-Chloro-1-(3-(4-(pyrimidin-2-yl)piperazine-1-carbonyl)benzyl)quinazoline-2,4(1H,3H)-dione;
5-Chloro-1-(3-(4-(cyclopentylcarbonyl)piperazine-1-carbonyl)benzyl)quinazoline-2,4(1H,3H)-dione;
5-Chloro-1-(3-(4-(cyclohexylcarbonyl)piperazine-1-carbonyl)benzyl)quinazoline-2,4(1H,3H)dione;
5-Chloro-1-(4-fluoro-3-(4-(pyrimidin-2-yl)piperazine-1-carbonyl)benzyl)quinazoline-2,4(1H,3H)-dione;
5-Chloro-1-(4-fluoro-3-(4-(cyclopentylcarbonyl)piperazine-1-carbonyl)benzyl)quinazoline-2,4(1H,3H)-dione;
5-Chloro-1-(4-fluoro-3-(4-(cyclohexylcarbonyl)piperazine-1-carbonyl)benzyl)quinazoline-2,4(1H,3H)-dione;
5-Chloro-1-(4-fluoro-3-(4-(pyridin-2-yl)piperazine-1-carbonyl)benzyl)quinazoline-2,4(1H,3H)-dione;
5-Chloro-1-(4-fluoro-3-(4-(thiophene-2-carbonyl)piperazine-1-carbonyl)benzyl)quinazoline-2,4(1H,3H)-dione;
5-Chloro-1-(6-fluoro-3-(4-(cyclohexylcarbonyl)piperazine-1-carbonyl)benzyl)quinazoline-2,4(1H,3H)-dione;
5-Chloro-1-(6-fluoro-3-(4-(cyclopentylcarbonyl)piperazine-1-carbonyl)benzyl)quinazoline-2,4(1H,3H)-dione;
5-Chloro-1-(6-fluoro-3-(4-(pyrimidin-2-yl)piperazine-1-carbonyl)benzyl)quinazoline-2,4(1H,3H)-dione;
6-Chloro-1-(3-(4-(pyrimidin-2-yl)piperazine-1-carbonyl)benzyl)quinazoline-2,4(1H,3H)-dione;
6-Chloro-1-(3-(4-(cyclopentylcarbonyl)piperazine-1-carbonyl)benzyl)quinazoline-2,4(1H,3H)-dione;
6-Chloro-1-(3-(4-(cyclohexylcarbonyl)piperazine-1-carbonyl)benzyl)quinazoline-2,4(1H,3H)dione;
6-Chloro-1-(3-(4-(pyridin-2-yl)piperazine-1-carbonyl)benzyl)quinazoline-2,4(1H,3H)-dione;
6-Chloro-1-(3-(4-(thiophene-2-carbonyl)piperazine-1-carbonyl)benzyl)quinazoline-2,4(1H,3H)-dione;
6-Chloro-1-(3-(4-(cyclobutylcarbonyl)piperazine-1-carbonyl)benzyl)quinazoline-2,4(1H,3H)-dione;
6-Chloro-1-(4-fluoro-3-(4-(pyrimidin-2-yl)piperazine-1-carbonyl)benzyl)quinazoline-2,4(1H,3H)-dione;
6-Chloro-1-(3-(4-(cyclopentylcarbonyl)piperazine-1-carbonyl)-4-fluorobenzyl)quinazoline-2,4(1H,3H)-dione;
6-Chloro-1-(3-(4-(cyclohexylcarbonyl)piperazine-1-carbonyl)-4-fluorobenzyl)quinazoline-2,4(1H,3H)-dione;
6-Chloro-1-(4-fluoro-3-(4-(pyridin-2-yl)piperazine-1-carbonyl)benzyl)quinazoline-2,4(1H,3H)-dione;
6-Chloro-1-(4-fluoro-3-(4-(thiophene-2-carbonyl)piperazine-1-carbonyl)benzyl)quinazoline-2,4(1H,3H)-dione;
6-Chloro-1-(4-fluoro-3-(4-(cyclopropylcarbonyl)piperazine-1-carbonyl)benzyl)quinazoline)-2,4(1H,3H)-dione;
6-Chloro-1-(6-fluoro-3-(4-(pyrimidin-2-yl)piperazine-1-carbonyl)benzyl)quinazoline-2,4(1H,3H)-dione;
6-Chloro-1-(6-fluoro-3-(4-(cyclopentylcarbonyl)piperazine-1-carbonyl)benzyl)quinazoline-2,4(1H,3H)-dione;
6-Chloro-1-(6-fluoro-3-(4-(cyclohexylcarbonyl)piperazine-1-carbonyl)benzyl)quinazoline-2,4(1H,3H)-dione;
6-Chloro-1-(6-fluoro-3-(4-(thiophene-2-carbonyl)piperazine-1-carbonyl)benzyl)quinazoline-2,4(1H,3H)-dione;
5-Fluoro-1-(3-(4-(pyrimidin-2-yl)piperazine-1-carbonyl)benzyl)quinazoline-2,4(1H,3H)-dione;
1-(3-(4-(Cyclopentylcarbonyl)piperazine-1-carbonyl)benzyl)quinazoline-2,4(1H,3H)-dione;
1-(3-(4-(Cyclohexylcarbonyl)piperazine-1-carbonyl)benzyl)quinazoline-2,4(1H,3H)-dione;
5-Fluoro-1-(3-(4-(pyridin-2-yl)piperazine-1-carbonyl)benzyl)quinazoline-2,4(1H,3H)-dione;
5-Fluoro-(1-(3-(4-(thiophene-2-carbonyl)piperazine-1-carbonyl)benzyl)quinazoline)-2,4(1H,3H)-dione;
5-Fluoro-1-(4-fluoro-3-(4-(pyrimidin-2-yl)piperazine-1-carbonyl)benzyl)quinazoline-2,4(1H,3H)-dione;
1-(4-fluoro-3-(4-(cyclopentylcarbonyl)piperazine-1-carbonyl)benzyl)quinazoline-2,4(1H,3H)-dione;
1-(4-fluoro-3-(4-(cyclohexylcarbonyl)piperazine-1-carbonyl)benzyl)quinazoline-2,4(1H,3H)-dione;
5-Fluoro-1-(4-fluoro-3-(4-(pyridin-2-yl)piperazine-1-carbonyl)benzyl)quinazoline-2,4(1H,3H)-dione;
5-Fluoro-1-(4-fluoro-3-(4-(thiophene-2-carbonyl)piperazine-1-carbonyl)benzyl)quinazoline-2,4(1H,3H)-dione;
1-(4-fluoro-3-(4-Benzoylpiperazine-1-carbonyl)benzyl)quinazoline-2,4(1H,3H)-dione;
1-(3-(4-(cyclohexylcarbonyl)piperazine-1-carbonyl)benzyl)quinazoline-2,4(1H,3H)-dione;
6-Fluoro-1-(3-(4-(pyrimidin-2-yl)piperazine-1-carbonyl)benzyl)quinazoline-2,4(1H,3H)-dione;
6-Fluoro-1-(3-(4-(pyridin-2-yl)piperazine-1-carbonyl)benzyl)quinazoline-2,4(1H,3H)-dione;
6-Fluoro-(1-(3-(4-(thiophene-2-carbonyl)piperazine-1-carbonyl)benzyl)quinazoline)-2,4(1H,3H)-dione;
1-(3-(4-(cyclohexylcarbonyl)piperazine-1-carbonyl)benzyl)quinazoline-2,4(1H,3H)-dione;
1-(3-(4-(cyclopentylcarbonyl)piperazine-1-carbonyl)benzyl)quinazoline-2,4(1H,3H)-dione;
6-Fluoro-1-(4-fluoro-3-(4-(pyrimidin-2-yl)piperazine-1-carbonyl)benzyl)quinazoline-2,4(1H,3H)-dione;
6-Fluoro-1-(4-fluoro-3-(4-(pyridin-2-yl)piperazine-1-carbonyl)benzyl)quinazoline-2,4(1H,3H)-dione;
6-Fluoro-1-(4-fluoro-3-(4-(thiophene-2-carbonyl)piperazine-1-carbonyl)benzyl)quinazoline-2,4(1H,3H)-dione;
1-(4-fluoro-3-(4-(cyclohexylcarbonyl)piperazine-1-carbonyl)benzyl)quinazoline-2,4(1H,3H)-dione;
1-(4-fluoro-3-(4-(cyclopentylcarbonyl)piperazine-1-carbonyl)benzyl)quinazoline-2,4(1H,3H)-dione;
6-Fluoro-1-(6-fluoro-3-(4-(pyrimidin-2-yl)piperazine-1-carbonyl)benzyl)quinazoline-2,4(1H,3H)-dione;
6-Fluoro-1-(6-fluoro-3-(4-(pyridin-2-yl)piperazine-1-carbonyl)benzyl)quinazoline-2,4(1H,3H)-dione;
6-Fluoro-1-(6-fluoro-3-(4-(thiophene-2-carbonyl)piperazine-1-carbonyl)benzyl)quinazoline-2,4(1H,3H)-dione;
1-(6-fluoro-3-(4-(cyclohexylcarbonyl)piperazine-1-carbonyl)benzyl)quinazoline-2,4(1H,3H)-dione;
1-(6-fluoro-3-(4-(cyclopentylcarbonyl)piperazine-1-carbonyl)benzyl)quinazoline-2,4(1H,3H)-dione;
5-Fluoro-1-(6-fluoro-3-(4-(pyrimidin-2-yl)piperazine-1-carbonyl)benzyl)quinazoline-2,4(1H,3H)-dione;
5-Fluoro-1-(6-fluoro-3-(4-(pyridin-2-yl)piperazine-1-carbonyl)benzyl)quinazoline-2,4(1H,3H)-dione;
5-Fluoro-1-(6-fluoro-3-(4-(thiophene-2-carbonyl)piperazine-1-carbonyl)benzyl)quinazoline-2,4(1H,3H)-dione;
1-(6-fluoro-3-(4-(cyclohexylcarbonyl)piperazine-1-carbonyl)benzyl)quinazoline-2,4(1H,3H)-dione;
1-(6-fluoro-3-(4-(cyclopentylcarbonyl)piperazine-1-carbonyl)benzyl)quinazoline-2,4(1H,3H)-dione;
5-Methyl-1-(3-(4-(pyrimidin-2-yl)piperazine-1-carbonyl)benzyl)quinazoline-2,4(1H,3H)-dione;
1-(3-(4-(cyclopentylcarbonyl)piperazine-1-carbonyl)benzyl)quinazoline-2,4(1H,3H)-dione;
7-Chloro-1-(3-(4-(pyrimidin-2-yl)piperazine-1-carbonyl)benzyl)quinazoline-2,4(1H,3H)-dione;
7-Chloro-1-(3-(4-(cyclopentylcarbonyl)piperazine-1-carbonyl)benzyl)quinazoline-2,4(1H,3H)-dione;
1-(4-fluoro-3-(4-(cyclopropylcarbonyl)piperazine-1-carbonyl)benzyl)quinazoline)-2,4(1H,3H)-dione;
6-Chloro-(4-fluoro-1-(3-(4-benzoylpiperazine-1-carbonyl)benzyl)quinazoline)-2,4(1H,3H)-dione;
1-(3-(4-(cyclobutylcarbonyl)piperazine-1-carbonyl)benzyl)quinazoline-2,4(1H,3H)-dione;
1-(4-fluoro-3-(4-(cyclobutylcarbonyl)piperazine-1-carbonyl)benzyl)quinazoline-2,4(1H,3H)-dione;
6-Chloro-1-(6-fluoro-3-(4-(cyclobutylcarbonyl)piperazine-1-carbonyl)benzyl)quinazoline-2,4(1H,3H)-dione;
5-Chloro-1-(4-fluoro-3-(4-(cyclobutylcarbonyl)piperazine-1-carbonyl)benzyl)quinazoline-2,4(1H,3H)-dione;
6-Chloro-1-(4-fluoro-3-(4-(cyclobutylcarbonyl)piperazine-1-carbonyl)benzyl)quinazoline-2,4(1H,3H)-dione;
5-Chloro-1-(6-fluoro-3-(4-(cyclobutylcarbonyl)piperazine-1-carbonyl)benzyl)quinazoline-2,4(1H,3H)-dione;
5-Chloro-1-(6-fluoro-3-(4-(thiophene-2-carbonyl)piperazine-1-carbonyl)benzyl)quinazoline-2,4(1H,3H)-dione;
5-Fluoro-1-(4-fluoro-3-(4-(furan-2-carbonyl)piperazine-1-carbonyl)benzyl)quinazoline-2,4(1H,3H)-dione;
5-Chloro-1-(6-fluoro-3-(4-(pyridin-2-yl)piperazine-1-carbonyl)benzyl)quinazoline-2,4(1H,3H)-dione;
6-Chloro-1-(6-fluoro-3-(4-(pyridin-2-yl)piperazine-1-carbonyl)benzyl)quinazoline-2,4(1H,3H)-dione;
6-Chloro-1-(5-fluoro-3-(4-(pyrimidin-2-yl)piperazine-1-carbonyl)benzyl)quinazoline-2,4(1H,3H)-dione;
5-Fluoro-1-(5-fluoro-3-(4-(pyrimidin-2-yl)piperazine-1-carbonyl)benzyl)quinazoline-2,4(1H,3H)-dione;
1-(3-(4-Cyclopentylpiperazine-1-carbonyl)benzyl)quinazoline-2,4(1H,3H)-dione;
1-(4-fluoro-3-(4-(cyclopropylcarbonyl)piperazine-1-carbonyl)benzyl)quinazoline-2,4(1H,3H)-dione;
1-(3-(4-(cyclobutylcarbonyl)piperazine-1-carbonyl)benzyl)quinazoline-2,4(1H,3H)-dione;
1-(4-fluoro-3-(4-(cyclobutylcarbonyl)piperazine-1-carbonyl)benzyl)quinazoline-2,4(1H,3H)-dione;
1-(4-fluoro-3-(4-benzoylpiperazine-1-carbonyl)benzyl)quinazoline-2,4(1H,3H)-dione;
1-(6-fluoro-3-(4-(cyclobutylcarbonyl)piperazine-1-carbonyl)benzyl)quinazoline-2,4(1H,3H)-dione;
1-(6-fluoro-3-(4-(cyclobutylcarbonyl)piperazine-1-carbonyl)benzyl)quinazoline-2,4(1H,3H)-dione;
5-Fluoro-1-(6-fluoro-3-(4-(furan-2-carbonyl)piperazine-1-carbonyl)benzyl)quinazoline-2,4(1H,3H)-dione;
6-Fluoro-1-(4-fluoro-3-(4-(furan-2-carbonyl)piperazine-1-carbonyl)benzyl)quinazoline-2,4(1H,3H)-dione;
6-Fluoro-1-(5-fluoro-3-(4-(pyrimidin-2-yl)piperazine-1-carbonyl)benzyl)quinazoline-2,4(1H,3H)-dione;
1-(6-chloro-3-(4-(pyrimidin-2-yl)piperazine-1-carbonyl)benzyl)quinazoline-2,4(1H,3H)-dione;
6-Fluoro-1-(4-fluoro-3-(4-(thiazol-2-yl)piperazine-1-carbonyl)benzyl)quinazoline-2,4(1H,3H)-dione;
5-Fluoro-1-(4-fluoro-3-(4-(thiazol-2-yl)piperazine-1-carbonyl)benzyl)quinazoline-2,4(1H,3H)-dione;
1-(4-Fluoro-3-(4-(thiazol-2-yl)piperazine-1-carbonyl)benzyl)quinazoline-2,4(1H,3H)-dione;
1-(3-(4-(Cyclohexylcarbamoyl)piperazine-1-carbonyl)benzyl)quinazoline-2,4(1H,3H)-dione;
1-(4-Fluoro-3-(4-(pyrimidin-2-yl)piperazine-1-carbonyl)benzyl)quinazoline-2,4(1H,3H)-dione hydrochloride;
5-Fluoro-1-(4-fluoro-3-(4-(pyrimidin-2-yl)piperazine-1-carbonyl)benzyl)quinazoline-2,4(1H,3H)-dione hydrochloride;
1-(3-(4-(Pyrimidin-2-yl)piperazine-1-carbonyl)benzyl)quinazoline-2,4(1H,3H)-dione hydrochloride;
1-(3-((4-(Pyridin-2-yl)piperazin-1-yl)methyl)benzyl)quinazoline-2,4(1H,3H)-dione;
1-(3-(Naphthalen-2-yl)acetamidobenzyl)quinazoline-2,4(1H,3H)-dione;
1-(3-(3,4-Dimethoxyphenyl)acetamidobenzyl)quinazoline-2,4(1H,3H)-dione;
1-(3-([1,2,4]Triazolo[4,3-a]pyridine-6-carbonyl)benzyl)quinazoline-2,4(1H,3H)-dione;
6-Fluoro-1-((2-(4-(pyrimidin-2-yl)piperazine-1-carbonyl)pyridin-6-yl)methyl)quinazoline-2,4(1H,3H)-dione;
5-Fluoro-1-(4-fluoro-3-(4-methoxybenzylcarbamoyl)benzyl)quinazoline-2,4(1H,3H)-dione;
1-(4-fluoro-3-(3-chlorobenzylcarbamoyl)benzyl)quinazoline-2,4(1H,3H)-dione;
1-(4-fluoro-3-(benzylcarbamoyl)benzyl)quinazoline-2,4(1H,3H)-dione;
5-Methoxy-1-(3-(4-(pyrimidin-2-yl)piperazine-1-carbonyl)benzyl)quinazoline-2,4(1H,3H)-dione;
6-Methoxy-1-(3-(4-(pyrimidin-2-yl)piperazine-1-carbonyl)benzyl)quinazoline-2,4(1H,3H)-dione;
1-(4-fluoro-3-(5-bromopyrimidin-2-aminocarbamoyl)benzyl)quinazoline-2,4(1H,3H)-dione;
1-(3-(4-(Pyrimidin-2-yl)piperazine-1-carbonyl)benzyl)quinazoline-2,4(1H,3H)-dione;
6,7-Ethylenedioxo-1-(3-(4-(pyrimidin-2-yl)piperazine-1-carbonyl)benzyl)quinazoline-2,4(1H,3H)-dione;
5-Fluoro-1-(6-methoxy-3-(4-(pyrimidin-2-yl)piperazine-1-carbonyl)benzyl)quinazoline-2,4(1H,3H)-dione;
7-Methoxy-1-(3-(4-(pyrimidin-2-yl)piperazine-1-carbonyl)benzyl)quinazoline-2,4(1H,3H)-dione;
5-Fluoro-1-(4-fluoro-3-(4-(tetrahydrofuran-2-carbonyl)piperazine-1-carbonyl)benzyl)quinazoline-2,4(1H,3H)-dione;
5-Fluoro-1-(4-nitro-3-(4-(pyrimidin-2-yl)piperazine-1-carbonyl)benzyl)quinazoline-2,4(1H,3H)-dione;
1-(4-fluoro-3-(4-cyclohexylpiperazine-1-carbonyl)benzyl)quinazoline-2,4(1H,3H)-dione;
5-Fluoro-1-(4-fluoro-3-(4-phenylpiperazine-1-carbonyl)benzyl)quinazoline-2,4(1H,3H)-dione;
5-Fluoro-1-(4-fluoro-3-(4-phenylpiperidine-1-carbonyl)benzyl)quinazoline-2,4(1H,3H)-dione;
1-(4-bromo-3-(4-(pyrimidin-2-yl)piperazine-1-carbonyl)benzyl)quinazoline-2,4(1H,3H)-dione;
6,7-Methylenedioxo-1-(3-(4-(pyrimidin-2-yl)piperazine-1-carbonyl)benzyl)quinazoline-2,4(1H,3H)-dione;
1-(4-fluoro-3-(4-(cyclohexylmethyl)piperazine-1-carbonyl)benzyl)quinazoline-2,4(1H,3H)-dione;
8-Fluoro-1-(3-(4-(pyrimidin-2-yl)piperazine-1-carbonyl)benzyl)quinazoline-2,4(1H,3H)-dione;
6-Amino-1-(3-(4-(pyrimidin-2-yl)piperazine-1-carbonyl)benzyl)quinazoline-2,4(1H,3H)-dione;
1-(2-(4-(Pyrimidin-2-yl)piperazine-1-carbonyl)benzyl)quinazoline-2,4(1H,3H)-dione;
8-Chloro-1-(3-(4-(pyrimidin-2-yl)piperazine-1-carbonyl)benzyl)quinazoline-2,4(1H,3H)-dione;
8-Methyl-1-(3-(4-(pyrimidin-2-yl)piperazine-1-carbonyl)benzyl)quinazoline-2,4(1H,3H)-dione;
8-Methoxy-1-(3-(4-(pyrimidin-2-yl)piperazine-1-carbonyl)benzyl)quinazoline-2,4(1H,3H)-dione; and
8-Hydroxy-1-(3-(4-(pyrimidin-2-yl)piperazine-1-carbonyl)benzyl)quinazoline-2,4(1H,3H)-dione;
or pharmaceutically acceptable salts, solvates or prodrugs thereof.

20. The kit of claim 13, wherein the amounts of the one or more pharmaceutical preparations of 5-fluoro-1-(4-fluoro-3-(4-(pyrimidin-2-yl)piperazine-1-carbonyl)benzyl)quinazoline-2,4(1H, 3H)-dione and one or more pharmaceutical preparations of TMZ in the kit are sufficient to provide the 5-fluoro-1-(4-fluoro-3-(4-(pyrimidin-2-yl)piperazine-1-carbonyl)benzyl)quinazoline-2,4(1H, 3H)-dione and TMZ for administration for at least 7 consecutive days, or at least 14 consecutive days or at least 21 consecutive days, or 28 days; or
the amounts of the one or more pharmaceutical preparations of 5-fluoro-1-(4-fluoro-3-(4-(pyrimidin-2-yl)piperazine-1-carbonyl)benzyl)quinazoline-2,4(1H, 3H)-dione and one or more pharmaceutical preparations of TMZ in the kit are sufficient to provide the 5-fluoro-1-(4-fluoro-3-(4-(pyrimidin-2-yl)piperazine-1-carbonyl)benzyl)quinazoline-2,4(1H, 3H)-dione and TMZ for administration for at least one cycle, or 2-8 cycles, wherein one cycle includes 28 consecutive days with 5-fluoro-1-(4-fluoro-3-(4-(pyrimidin-2-yl)piperazine-1-carbonyl)benzyl)quinazoline-2,4(1H, 3H)-dione being administered for all 28 days and TMZ being administered for 21 consecutive days.

21. The compound preparation of claim 14, wherein the compound preparation contains:
5-fluoro-1-(4-fluoro-3-(4-(pyrimidin-2-yl)piperazine-1-carbonyl)benzyl)quinazoline-2,4(1H,3H)-dione in a dose of 40 mg and TMZ in a dose of 20 mg, or
5-fluoro-1-(4-fluoro-3-(4-(pyrimidin-2-yl)piperazine-1-carbonyl)benzyl)quinazoline-2,4(1H,3H)-dione in a dose of 60 mg and TMZ in a dose of 20 mg, or
5-fluoro-1-(4-fluoro-3-(4-(pyrimidin-2-yl)piperazine-1-carbonyl)benzyl)quinazoline-2,4(1H,3H)-dione in a dose of 80 mg and TMZ in a dose of 20 mg, or
5-fluoro-1-(4-fluoro-3-(4-(pyrimidin-2-yl)piperazine-1-carbonyl)benzyl)quinazoline-2,4(1H,3H)-dione in a dose of 80 mg and TMZ in a dose of 30 mg, or
5-fluoro-1-(4-fluoro-3-(4-(pyrimidin-2-yl)piperazine-1-carbonyl)benzyl)quinazoline-2,4(1H,3H)-dione in a dose of 40 mg and TMZ in a dose of 10 mg, or
5-fluoro-1-(4-fluoro-3-(4-(pyrimidin-2-yl)piperazine-1-carbonyl)benzyl)quinazoline-2,4(1H,3H)-dione in a dose of 20 mg and TMZ in a dose of 10 mg.
